(19) United States
(12) Patent Application Publication
(10) Pub. No.: US 2020/0056198 A1
(43) Pub. Date: Feb. 20, 2020

(54) ENZYMES FOR THE SYNTHESIS OF ACETYL-TRIACYLGLYCEROLS

(71) Applicant: Kansas State University Research Foundation, Manhattan, KS (US)

(72) Inventors: Timothy Durrett, Manhattan, KS (US); Tam Nguyen Thu Tran, Dallas, TX (US); Catherine Kornacki, St. Louis, MO (US)

(21) Appl. No.: 16/608,533

(22) PCT Filed: Apr. 26, 2018
(86) PCT No.: PCT/US2018/029590

§ 371 Date: Oct. 25, 2019

Related U.S. Application Data
(60) Provisional application No. 62/490,114, filed on Apr. 26, 2017.

(51) Int. Cl. C12N 15/82; C12N 9/10; C12P 7/64

(52) U.S. Cl. CPC C12N 15/8247; C12N 15/8218; C12Y 203/0102; C12P 7/6445; C12N 9/1029

(57) ABSTRACT

The present invention relates to diacylglycerol acyltransferase genes and proteins, and methods of their use. In particular, the invention describes genes encoding proteins having increased diacylglycerol acetyltransferase activity compared to prior art proteins, specifically for transferring an acetyl group to a diacylglycerol substrate to form acetyl-Triacylglycerols (acetyl-TAGS), for example, a 3-acetyl-1,2-diacyl-sn-glycerol. The present invention encompasses both native and recombinant wild-type forms of the transferase, as well as mutants and variant forms. The present invention also relates to methods of using the diacylglycerol acyltransferase genes and proteins, including their expression in transgenic organisms at commercially viable levels, for increasing production of 3-acetyl-1,2-diacyl-sn-glycerols in plant oils and altering the composition of oils produced by microorganisms, such as yeast, by increasing acetyl-TAG production. Additionally, oils produced by methods of the present inventions comprising genes and proteins are contemplated for use as biodiesel fuel, in polymer production and as naturally produced food oils with reduced calories.

### CROSS-REFERENCE TO RELATED APPLICATIONS

The present application claims the priority benefit of U.S. Provisional Patent Application Ser. No. 62/490,114, filed Apr. 26, 2017, entitled IMPROVED ENZYMES FOR THE SYNTHESIS OF ACETYL-TRIACYLGLYCEROLS, incorporated by reference in its entirety herein.

### FEDERALLY SPONSORED RESEARCH OR DEVELOPMENT

This invention was made with government support under Contract No. EPS-0903806 awarded by the National Science Foundation, Contract No. DE-SC0012459 awarded by the United States Department of Energy, and Contract No. 2015-67013-22815 awarded by the United States Department of Agriculture. The government has certain rights in the invention.

### SEQUENCE LISTING

The following application contains a sequence listing in computer readable format (CRF), submitted as a text file in ASCII format entitled “Sequence_Listing,” created on Apr. 26, 2018, as 66 KB. The content of the CRF is hereby incorporated by reference.

### BACKGROUND OF THE INVENTION

### Field of the Invention

The invention comprises new enzymes capable of synthesizing acetyl-TAGs in transgenic plants and yeast at higher levels than existing technology.

### Description of Related Art

Acetyl-TAGs are atypical triacylglycerols (TAGs) that contain an acetate group instead of a fatty acid at the sn-3 position of the glycerol backbone (FIG. 1). This acetate group imparts different chemical and physical properties making acetyl-TAGs valuable in applications where regular vegetable oils are unsuitable. For example, acetyl-TAGs possess a kinematic viscosity that is approximately 40% lower than that of lcTAGs. Additionally, acetyl-TAGs possess superior cold temperature properties. Visual observations reveal that acetyl-TAGs remain liquid at temperatures that cause other oils to solidify. Indeed, the lower melting point of these oils is used by some insects as a thermotolerance mechanism. Differential scanning calorimetry has also confirmed the improved cold temperature properties of transgenically produced acetyl-TAGs. These and other altered properties make acetyl-TAGs valuable for many applications that are not suitable for conventional plant oils without subsequent processing. For example, acetyl-TAGs could be used as an improved Pure Plant Oil (PPO) biofuel or as cold-resistant biodegradable lubricants.

In addition to the industrial applications noted above, acetyl-TAGs are functionally equivalent to chemically synthesized compounds called ACETEM (acetic acid esters of mono- and diacylglycerols). ACETEM are used in a variety of applications, including as emulsifiers, lubricants and emulsifiers in the food industry.

Seeds of Euonymus alatus (Burning Bush) accumulate acetyl-TAGs as the major component of their storage oil. Previously, an acetyltransferase, EaDAcT, from E. alatus that synthesizes acetyl-TAGs from DAG and acetyl-CoA in vitro and results in up to 45 mol % acetyl-TAG when expressed in wild-type Arabidopsis seeds was identified (U.S. Pat. No. 9,328,335, incorporated by reference herein). Subsequent expression of EaDAcT in the oil seed crop Camelina sativa combined with the suppression of the competing DGAT1 enzyme resulted in acetyl-TAG levels up to 85 mol % in the best transgenic lines. At the time, this represented the highest levels of an unusual lipid molecule synthesized in a transgenic seed.

### SUMMARY OF THE INVENTION

The present disclosure relates to improved production of acetyl-TriAcylGlycerols (acetyl-TAGS) by biological organisms (bio-production), for example for use in biofuels. In particular, described herein are systems and methods for producing acetyl-TAGS using transgenic organisms expressing the diacylglycerol acyltransferase (DAcT) gene derived from Euonymus fortunei or Euonymus kiautschovicus (EfDAcT and EkDAcT, respectively). In one or more embodiments, EfDAcT and/or EkDAcT is expressed in yeast and oil-seed crop plants, such as soybean plants, rapeseed plants, camelina plants, pennycress (Thlaspi arvense) plants, Jatropha plants, and the like, for use in providing biofuels. In other embodiments, EfDAcT and/or EkDAcT is expressed in oil-seed crop plants, such as soybean plants, Canola-quality plants, and the like, for use as commercial sources of oil used in food preparation. In further embodiments, EfDAcT and/or EkDAcT is expressed in yeast and oil-seed crop plants for providing novel TAG monomers for use in commercial reactions to provide a more refined control of polymers and polymer properties for commercial applications.

In one or more embodiments, an isolated nucleic acid sequence operably linked to a heterologous promoter is described herein, wherein the nucleic acid encodes a short chain acyl-CoA diacylglycerol acyltransferase plant protein. In certain embodiments, the protein is at least 95% identical to SEQ ID NO:1 or SEQ ID NO:2. In some embodiments, the plant is selected from the group consisting of Celastraceae, Lardizabalaceae, Ranunculaceae, Rosaceae, and Vitaceae. In some embodiments, the plant is selected from the group consisting of Euonymus, Maytenus, Akebia, Adonis, Sorbus and Vilis species. In some embodiments, the plant is an Euonymus fortunei or Euonymus kiautschovicus plant. In one embodiment, the isolated nucleic acid sequence is at least 95% identical to SEQ ID NO:18 or SEQ ID NO: 19. Accordingly, in other embodiments, the isolated nucleic acid sequence is at least 95%, 98%, 99% (or more) identical to SEQ ID NO:18 or SEQ ID NO:19. As noted above, the isolated nucleic acid sequence encodes a protein at least 95% identical to SEQ ID NO:1 or SEQ ID NO:2. Accordingly, in other embodiments, the protein comprises a polypeptide at least 95%, 98%, 99% (or more) identical to SEQ ID NO:1 or SEQ ID NO:2. In some embodiments, the protein is capable of acetylating a diacylglycerol substrate comprising a fatty acid to form an acetyltriacylglycerol. In some embodiments, the fatty acid is selected from the group consisting of butyrate, caproate, caprylate, caprate, laurate, myristate, palmitate, palmitoleate, stearate, oleate, linoleate, linolenate, arachidonate, eicosenoate, eicosadienoate, and erucate. In some embodiments, the diacylglycerol substrate is selected from the group consisting of 1,2-dipalmitoyl-glycerol, 1-palmitoyl-2-oleoyl-glycerol, 2-palmitoyl-1-oleoyl-glycerol, and 1,2-dioleoyl-glycerol. In some embodiments, the diacylglycerol acyltransferase protein is capable of acylating the diacylglycerol substrate with an acyl-coenzyme A substrate. In some embodiments, the acyl-coenzyme A substrate is selected from the group consisting of a two carbon acyl-coenzyme A, a three carbon acyl-coenzyme A, a short chain acyl-coenzyme A, and a medium chain acyl-coenzyme A. In some embodiments, the acyl-coenzyme A substrate is selected from the group consisting of an acetyl-coenzyme A, propionyl-coenzyme A, butyryl-coenzyme A, hexanoyl-coenzyme A, octanoyl-coenzyme A, and deconyl-coenzyme A.

The present invention further provides a vector comprising the isolated nucleic acid sequence. The present inventions are not limited to a particular vector. Indeed a variety of vectors are contemplated, including but not limited to an expression vector, a vector active in a plant cell, a vector active in a fungal cell, a vector active in a yeast cell, a vector active in an algal cell, etc. In some embodiments, a vector is adapted for use in an Agrobacterium mediated transfection. In some embodiments, a vector active in a plant is a p2S.GATEWAY vector or a pBinGlyRed3 vector. In some embodiments, a vector active in a yeast cell is a pYES2/CT vector. As noted above, the nucleic acid sequence is operably linked to a heterologous promoter. The present inventions are not limited to a particular promoter. Indeed, a variety of promoters are contemplated, including but not limited to diacylglycerol acyltransferase promoters, promoters active in a plant cell, promoters active in a seed, promoters active in a fungal cell, promoters active in a yeast cell, promoters active in an algal cell, promoters from an Euonymus fortunei or Euonymus kiautschovicus plant, promoters from a crop oil plant, etc. In some embodiments, the heterologous promoter is a tissue specific promoter. In some embodiments, the heterologous promoter is a seed specific promoter. The present inventions are not limited to a particular promoter active in a seed. Indeed, a variety of promoters active in a seed are contemplated, including but not limited to a 2S promoter sequence, seed storage protein promoters, such as a phaseolin promoter, a napin promoter, an oleosin promoter, et cetera.

The present invention further provides a host cell comprising the vector. In some embodiments, the host cell is selected from the group consisting of a plant cell and a microorganism. In some embodiments, the plant cell is selected from the group consisting of an edible crop plant cell, an oil seed crop plant cell, a seed cell, a pollen cell, an ovule cell, mesenchymal cell, meristem cell, an endosperm cell, a male reproductive cell, a female reproductive cell, and an embryo cell. In some embodiments, the plant cell is selected from the group consisting of a Jatropha plant, an oil crop plant, a palm oil plant, and an alga. In some embodiments, the plant cell is selected from the group consisting of Brassica plants and Brassicaceae plants. In some embodiments, the plant cell is selected from the group consisting of Arabidopsis plants, Camelina plants, and crambe plants. In certain such embodiments, the plant cell is Camelia sativa. In some embodiments, the microorganism is a fungus cell. In one embodiment, the fungus cell is a yeast cell. In some embodiments, the host cell has lower long chain-triacylglycerol production. In some embodiments, the host cell has low long chain-triacylglycerol production. In some embodiments, the host cell expresses a mutant fatty acid elongase 1 gene resulting in lower very long chain fatty acid production. In some embodiments, the host cell expresses a mutant fatty acid elongase 1 gene resulting in low very long chain-fatty acid production. In some embodiments, the mutant fatty acid elongase 1 gene encodes a mutant FAE1 protein. In some embodiments, the mutant fatty acid elongase 1 gene has a stop codon resulting in a truncation mutant FAE1 protein. In some embodiments, the host cell comprises at least one silenced fatty acid elongase 1 gene, wherein the gene is silenced due to a mutation which results in lowered expression of the gene in the host cell. In some embodiments, the host cell comprises at least one silenced fatty acid elongase 1 gene, wherein the silencing results in low very long chain fatty acid production in the host cell. In some embodiments, the host cell comprises at least one silenced triacylglycerol synthesis gene, wherein the gene is silenced due to a mutation that results in lowered expression of the gene. In some embodiments, the host cell comprises at least one silenced triacylglycerol synthesis gene, wherein the gene is silenced due to RNAi for targeting the silenced gene. In some embodiments, the silenced triacylglycerol synthesis gene is selected from the group consisting of diacylglycerol acyltransferase 1, diacylglycerol acyltransferase 2, and phospholipid: diacylglycerol acyltransferase.

The present invention further provides an oil produced by the host cell comprising a triacylglycerol consisting of two acyl groups and an acetyl group. In some embodiments, the triacylglycerol comprises a 3-acetyl-1,2-diacyl-sn-glycerol. In some embodiments, the oil has lower caloric energy than an oil isolated from a nontransformed host cell. In one embodiment, the triacylglycerol ranges from 1%-99% (molar ratio) of total triacylglycerols in the isolated oil. In some embodiments, the triacylglycerol is at least 90% (molar ratio) of total triacylglycerols in the oil.

The present invention further provides a plant, wherein the plant comprises a heterologous plant nucleic acid sequence operably linked to a heterologous promoter, wherein the nucleic acid sequence encodes an acetyl-CoA diacylglycerol acyltransferase protein. In some embodiments, the protein is at least 95% identical to SEQ ID NO:1 or SEQ ID NO:2. In some embodiments, the plant has low long chain-triacylglycerol production. In some embodiments, the plant further comprises a mutant gene, wherein the mutant gene is selected from the group consisting of a diacylglycerol acyltransferase 1, diacylglycerol acyltransferase 2, and phospholipid; diacylglycerol acyltransferase gene. In some embodiments, the plant comprises at least one silenced triacylglycerol synthesis gene, wherein the gene is silenced due to RNAi for targeting the silenced gene. In some embodiments, the silenced triacylglycerol synthesis gene is selected from the group consisting of diacylglycerol acyltransferase 1, diacylglycerol acyltransferase 2, and phospholipid: diacylglycerol acyltransferase.

The present invention further provides a seed, wherein the seed comprises a heterologous plant nucleic acid sequence operably linked to a heterologous promoter, wherein the nucleic acid encodes an acetyl-CoA diacylglycerol acyltransferase protein. In some embodiments, the protein is at least 95% identical to SEQ ID NO:1 or SEQ ID NO:2.

The present invention provides compositions comprising an isolated nucleic acid sequence operably linked to a heterologous promoter, wherein the nucleic acid encodes a short chain acyl-CoA diacylglycerol acyltransferase plant protein. In some embodiments, the protein is at least 95% identical to SEQ ID NO:1 or SEQ ID NO:2. In some embodiments, the protein is capable of acetylating a diacylglycerol substrate comprising a fatty acid to form an acetyltriacylglycerol. In some embodiments, the fatty acid is selected from the group consisting of butyrate, caproate, caprylate, caprate, laurate, myristate, palmitate, palmitoleate, stearate, oleate, linoleate, linolenate, arachidonate, eicosenoate, eicosadienoate, and erucate. The present inventions are not limited to a particular diacylglycerol substrate. Indeed a variety of substrates are contemplated, including but not limited to 1,2-dipalmitoyl-glycerol, 1-palmitoyl-2 oleoyl-glycerol, 2-palmitoyl-1-oleoyl-1,2-dioleoyl-glycerol, and the like. In some embodiments, the diacylglycerol acyltransferase protein is capable of acylating the diacylglycerol substrate with an acyl-coenzyme A substrate. The present inventions are not limited to a particular acyl-coenzyme A substrate. Indeed a variety of acyl-coenzyme A substrates are contemplated, including but not limited to a two carbon acyl coenzyme A, a three carbon acyl-coenzyme A, a short chain acyl-coenzyme A, a medium chain acyl-coenzyme A, and the like. In some embodiments, the acyl-coenzyme A substrate is selected from the group consisting of an acetyl-coenzyme A, propionyl-coenzyme A, butyryl-coenzyme A, hexanoyl-coenzyme A, octanoyl-coenzyme A, and deconyl-coenzyme A. As noted above, the protein is at least 95% identical to SEQ ID NO:1 or SEQ ID NO:2. Accordingly, in other embodiments, the protein comprises a polypeptide at least 95%, 98%, 99% (or more) identical to SEQ ID NO:1 or SEQ ID NO:2. In some embodiments, the plant is selected from the group consisting of Celastraceae, Lardizabalaceae, Ranunculaceae, Rosaceae, and Vitaceae. In some embodiments, the plant is selected from the group consisting of Euonymus, Maytenus, Akebia, Adonis, Sorbus, and Vitis species. In some embodiments, the plant is an Euonymus fortunei or Euonymus kiautschovicus plant. As noted above, the present invention provides compositions comprising an isolated nucleic acid sequence operably linked to a heterologous promoter. The present inventions are not limited to a particular promoter. Indeed a variety of promoters are contemplated, including but not limited to diacylglycerol acyltransferase promoters, promoters active in a plant cell, promoters active in a seed, promoters active in a fungal cell, promoters active in a yeast cell, promoters active in an algal cell, promoters from an Euonymus fortunei or Euonymus kiautschovicus plant, promoters from a crop oil plant, etc. In some embodiments, the heterologous promoter is a tissue specific promoter. In some embodiments, the heterologous promoter is a seed specific promoter. The present inventions are not limited to a particular promoter active in a seed. Indeed a variety of promoters active in a seed are contemplated, including but not limited to a 2S promoter sequence, seed storage protein promoters, such as a phaseolin promoter, a napin promoter, an oleosin promoter, etc.

Additionally, in some embodiments, the inventions provide compositions comprising a vector further comprising the nucleic acid sequence. The present inventions are not limited to a particular vector. Indeed a variety of vectors are contemplated, including but not limited to an expression vector, a vector active in a plant cell, a vector active in a fungal cell, a vector active in a yeast cell, a vector active in an algal cell, etc. In some embodiments, a vector is adapted for use in an Agrobacterium mediated transfection. In some embodiments, a vector active in a plant is a to p2S.GATEWAY vector or a pBinGlyRed3 vector. In some embodiments, a vector active in a yeast cell is a pYES-2/CT vector.

Additionally, in some embodiments, the inventions provide a host cell comprising the vector of the present inventions. In some embodiments, the host cell is selected from the group consisting of a plant cell and a microorganism. The present inventions are not limited to a particular plant cell. Indeed a variety of plant cells are contemplated, including but not limited to an edible crop plant cell and an oil crop plant cell. In some embodiments, the host cell is selected from the group consisting of a mesenchymal cell, meristem cell, an endosperm cell, a pollen cell, a seed cell, oil seed plant cell, a male reproductive cell, a female reproductive cell, and an embryo cell. In some embodiments, the plant cell includes but is not limited to a Jatropha plant cell, an oil crop plant cell, a palm oil plant cell, an alga cell, etc. In some embodiments, the plant cell includes but is not limited to a Brassica plant cell and Brassicaceae plant cell. In some embodiments, the plant cell includes but is not limited to an Arabidopsis plant cell, Camelina plant cell, crambe plant cell, etc. In certain such embodiments, the plant cell is a Camelina sativa plant cell. In some embodiments, the microorganism is a fungus cell. The present inventions are not limited to a particular fungus cell. Indeed a variety of fungus cells are contemplated, including but not limited to a yeast cell, an oleaginous fungal cell, an oleaginous yeast cell, etc. In yet further embodiments, the host cell has low long chain-triacylglycerol production. In one embodiment, low long chain-triacylglycerol production has altered substrate availability. In one embodiment, altered substrate availability is the result of reduced DGAT gene expression. In one embodiment, altered substrate availability is the result of reduced DGAT protein expression. In one embodiment, altered substrate availability is the result of reduced PDAT gene expression. In one embodiment, altered substrate availability is the result of reduced PDAT protein expression. In other embodiments, altered substrate availability is the result of changes in genes controlling fatty acid production, such as citrate lyase, fatty acid elongase gene 1 (FAE1), and the like. In some embodiments, the host cell comprises at least one silenced fatty acid elongase gene 1 (fae1 gene). In some embodiments, the host cell comprises at least one mutant fatty acid elongase 1 (fae1) gene. In some embodiments, the host cell comprises at least one fatty acid elongase 1 (fae1) gene comprising a mutation for reducing expression of a functional FAE1 protein. In some embodiments, the host cell comprises at least one mutant fatty acid elongase 1 (fae1) protein which results in low amounts of very long chain fatty acids in the cell. In an exemplary embodiment, the host cell is a CB25 Arabidopsis plant line cell. In some embodiments, the host cell comprises at least one silenced triacylglycerol synthesis gene, wherein the silenced gene has reduced expression when compared to the gene in a wild-type plant. It is not meant to limit the method of reduction in expression of the triacylglycerol synthesis gene. Indeed a variety of methods are contemplated including identifying a natural mutation in the gene, inducing a mutation in the gene, engineering the reduction in expression of the gene and the like. In some embodiments, the expression is reduced due to a mutation that results in lowered expression of the gene. In some embodiments, the expression is reduced due to expression of a truncation mutant, such as a fatty acid elongase 1 (fae1) gene comprising a stop codon within the coding region. In some embodiments, the expression is reduced due to expression of an RNAi molecule for silencing the gene. In some embodiments, the silenced triacylglycerol synthesis gene is selected from the group consisting of diacylglycerol acyltransferase 1, diacylglycerol acyltransferase 2, phospholipid:diacylglycerol acyltransferase, and the like.

The present invention provides a composition comprising a host cell, wherein the host cell comprises a heterologous plant nucleic acid sequence operably linked to a heterologous promoter, wherein the nucleic acid encodes an acetyl-CoA diacylglycerol acyltransferase protein. In some embodiments, the protein is is at least 95% identical to SEQ ID NO:1 or SEQ ID NO:2. In some embodiments, the host cell is an oil seed plant cell, mesenchymal cell, meristem cell, an endosperm cell, a pollen cell, a seed cell, a male reproductive cell, a female reproductive cell, an ovule cell, and an embryo cell, etc. In some embodiments, the composition further comprises acetyltriacylglycerol. In some embodiments, the host cell further comprises acetyltriacylglycerol. In some embodiments, the acetyltriacylglycerol comprises a 3-acetyl-1,2-diacyl-sn-glycerol.

Additionally, in some embodiments, the inventions provide oil produced by the host cell of the present inventions comprising a triacylglycerol molecule consisting of two acyl groups and an acetyl group. In preferred embodiments, the triacylglycerol is an acetyltriacylglycerol. In some embodiments, the triacylglycerol comprises a 3-acetyl-1,2-diacyl-sn-glycerol. In some embodiments, the oil has a lower caloric energy than oil isolated from a nontransformed host cell. In some embodiments, the lower caloric energy is a lower energy per gram. In some embodiments, the acetyltriacylglycerol range from 1%-99% (molar ratio by dry weight) of total triacylglycerols in the isolated oil. In some embodiments, the acetyltriacylglycerol is at least 90% (molar ratio) of total triacylglycerols in the oil. Accordingly, in other embodiments, the molar ratio of acetyltriacylglycerol is at least 90%, 95%, 98%, 99% (or more) of total triacylglycerols in the oil.

Additionally, in some embodiments, the inventions provide a plant comprising a heterologous plant nucleic acid sequence operably linked to a heterologous promoter, wherein the nucleic acid encodes an acetyl-CoA diacylglycerol acyltransferase protein. In some embodiments, the protein is at least 95% identical to SEQ ID NO:1 or SEQ ID NO:2. The present inventions are not limited to a particular plant. Indeed a variety of plants are contemplated, including but not limited to a Camelina plant (such as Camelina sativa), pennycress, a Jatropha plant, an oil crop plant, a palm oil plant, and an alga. In some embodiments, the plant has low long chain-triacylglycerol production. In some embodiments, the plant further comprises at least one silenced fatty acid elongase 1 gene, wherein the silenced gene has reduced expression of a functional FAE1 protein when compared to the gene in a wild-type plant. In some embodiments, the plant is a CB25 Arabidopsis plant. In some embodiments, the plant further comprises at least one silenced triacylglycerol synthesis gene, wherein the silenced gene has reduced expression when compared to the gene in a wild-type plant. It is not meant to limit the method of reduction in expression of the triacylglycerol synthesis gene. Indeed a variety of methods are contemplated including identifying a natural mutation in the gene, inducing a mutation in the gene, engineering the reduction in expression of the gene and the like. In some embodiments, the expression is reduced due to a mutation that results in lowered expression of the gene. In some embodiments, the expression is reduced due to expression of an RNAi molecule for silencing the gene. In some embodiments, the silenced triacylglycerol synthesis gene is selected from the group consisting of diacylglycerol acyltransferase 1, diacylglycerol acyltransferase 2, phospholipid: diacylglycerol acyltransferase, and the like. In some embodiments, the plant is a plant cell. In some embodiments, the plant cell includes but is not limited to a Brassica plant cell and Brassicaceae plant cell. In some embodiments, the plant cell includes but is not limited to an Arabidopsis plant cell, Camelina plant cell, Crambe plant cells, etc.

The present invention is not limited to any particular acetyl-TAG producing plant, i.e. an acetyl-TAG plant producing a seed comprising acetyl-TAGS. Indeed, a variety of acetyl-TAG producing plants are contemplated, including but not limited to an acetyl-TAG producing plant, an acetyl-TAG producing plant comprising an agronomically desirable trait, a progeny plant of a transgenic acetyl-TAG producing plant, an acetyl-TAG producing plant that is an agronomically desirable plant, an acetyl-TAG producing plant that is a commercially desirable plant, and an acetyl-TAG producing plant that is a commercially desirable cultivar.

Additionally, in some embodiments, the inventions provide a seed comprising a plant nucleic acid sequence operably linked to a heterologous promoter, wherein the nucleic acid encodes an acetyl-CoA diacylglycerol acyltransferase protein. In some embodiments, the protein is at least 95% identical to SEQ ID NO:1 or SEQ ID NO:2. The present inventions are not limited to a particular seed. Indeed, a variety of seeds are contemplated, including but not limited to a Jatropha plant seed, an oil crop plant seed, a palm oil plant seed, and an alga seed. In some embodiments, the seed includes but is not limited to a Brassica plant seed and Brassicaceae plant seed. In some embodiments, the seed includes but is not limited to an Arabidopsis plant seed, Camelina plant seed, Crambe plant seed, etc. In certain such embodiments, the seed is a Camelina sativa plant seed.

Additionally, in some embodiments, the inventions provide a composition comprising a seed, wherein the seed comprising a heterologous plant nucleic acid sequence encoding a protein at least 95% identical to SEQ ID NO:1 or SEQ ID NO:2 and capable of forming acetyltriacylglycerol molecules.

In addition, the present invention provides methods, comprising providing an isolated nucleic acid sequence encoding a protein at least 95% identical to SEQ ID NO:1 or SEQ ID NO:2 and capable of forming an acetyltriacylglycerol and a host cell, transforming the host cell with the isolated nucleic acid sequence such that the nucleic acid expresses the protein, and isolating an acetyltriacylglycerol from the host cell. In some embodiments, the acetyltriacylglycerol is a 3-acetyl-1,2-diacyl-sn-glycerol. In some embodiments, the host cell is selected from a fungal cell, an alga cell and a plant cell. In some embodiments, the isolating comprises lipid extraction. In some embodiments, the methods further comprise incubating the transformed cell in a medium. In some embodiments, the host cell further comprises a heterologous gene and expresses the heterologous gene under conditions increasing a substrate for the acetyl-CoA diacylglycerol acyltransferase protein. The present inventions are not limited to a particular heterologous gene. Indeed, a variety of heterologous genes are contemplated, including but not limited to genes for altering fatty acid synthesis, fatty acid synthesizing enzymes, and the like. In some embodiments, the heterologous gene reduces long chain fatty acid synthesis. In some embodiments, expression of the heterologous gene reduces long chain fatty acid synthesis. In some embodiments, the heterologous gene encodes an ATP-citrate lyase enzyme. In some embodiments, the heterologous gene encodes an acyl-ACP thioesterase (FatB2) protein. In some embodiments, the heterologous gene encodes a FAE1 mutant protein. In some embodiments, the heterologous gene encodes a FAE1 truncation mutant protein. In some embodiments, the host cell further comprises an inhibitory heterologous nucleic acid capable of interfering with the production of a long-chain-triacylglycerol molecule for increasing amounts of isolated acetyltriacylglycerol. The present inventions are not limited to a particular inhibitory heterologous nucleic acid. Indeed a variety of inhibitory heterologous nucleic acids are contemplated, including but not limited to a diacylglycerol acyltransferase 1 gene, diacylglycerol acyltransferase 2 gene, and phospholipid:diacylglycerol acyltransferase gene. In some embodiments, the inhibitory nucleic acid is a siRNA. In some embodiments, the production of long chain-triacylglycerol molecules is reduced. In some embodiments, the host cell has lower long chain-triacylglycerol production. In some embodiments, the host cell has low long chain-triacylglycerol production. In some embodiments, the host cell expresses a mutant fatty acid elongase gene 1 gene resulting in lower long chain-triacylglycerol production. In some embodiments, the host cell expresses a mutant fatty acid elongase gene 1 gene resulting in low long chain-triacylglycerol production. In some embodiments, the acetyltriacylglycerol comprises a 3-acetyl-1,2-diacyl-sn-glycerol.

In addition, the present invention provides methods, comprising providing a plant part comprising a heterologous nucleic acid sequence encoding a protein at least 95% identical to SEQ ID NO:1 or SEQ ID NO:2 and capable of forming acetyltriacylglycerol, growing the plant part under conditions such that the nucleic acid expresses the protein wherein acetyltriacylglycerol production is increased, and isolating acetyltriacylglycerol from the plant part. In some embodiments, the acetyltriacylglycerol is 3-acetyl-1,2-diacyl-sn-glycerol. In some embodiments, the plant part is selected from a seed, aril, stem, leaf, tubers, mesocarp, pericarp, exocarp, cell wall, and frond. In some embodiments, the host cell further comprises an inhibitory heterologous nucleic acid capable of interfering with the production of a long-chain-triacylglycerol molecule for increasing amounts of isolated 3-acetyl-1,2-diacyl-sn-glycerol. In some embodiments, the inhibitory nucleic acid is selected from the group consisting of a diacylglycerol acyltransferase 1 gene, diacylglycerol acyltransferase 2 gene, and a phospholipid:diacylglycerol acyltransferase gene. In some embodiments, the inhibitory nucleic acid is a siRNA. In some embodiments, the production of long-chain-triacylglycerol molecules is reduced. In some embodiments, the plant part further comprises a heterologous nucleic acid sequence encoding a protein capable of increasing a substrate for the acetyl-CoA diacylglycerol acyltransferase protein. In some embodiments, the heterologous nucleic acid encodes a truncated fatty acid elongase 1 mutant protein. In some embodiments, the heterologous nucleic acid is a mutant fatty acid elongase 1 gene. In some embodiments, the plant part further comprises a heterologous acyl nucleic acid sequence encoding a protein capable of increasing a substrate for the acetyl-CoA diacylglycerol acyltransferase protein. In some embodiments, the heterologous gene encodes an ATP-citrate lyase enzyme. In some embodiments, the heterologous gene encodes an acyl-ACP thioesterase protein. In some embodiments, the heterologous gene encodes a fatty acid elongase 1 protein. In some embodiments, the substrate is selected from the group consisting of a short chain acyl-CoA and medium chain acyl-CoA. In some embodiments, the substrate is selected from the group consisting of an acetyl-CoA, butyryl-CoA, hexanoyl-CoA, octanoyl-CoA, and deconyl-CoA.

In addition, the present invention provides methods, comprising isolating oil from a host cell expressing a heterologous gene encoding a protein at least 95% identical to SEQ ID NO:1 or SEQ ID NO:2 and capable of making an acetyltriacylglycerol, wherein the oil comprises a triacylglycerol consisting of two functionalized acyl groups and an acetyl group, and using the oil in an application selected from the group consisting of lubricant, biofuel, spray coating, food oil, in food processing, and thermoplastic polymer products.

In addition, the present invention provides methods comprising isolating oil from a host cell expressing a heterologous gene encoding a protein at least 95% identical to SEQ ID NO:1 or SEQ ID NO:2 and capable of making an acetyltriacylglycerol, wherein the oil comprises a triacylglycerol consisting of two acyl groups and an acetyl group, and using the oil in an application selected from the group consisting of lubricant, biofuel, spray coating, food oil, in food processing, and thermoplastic polymer products.

In addition, the present invention provides methods comprising providing a host plant capable of producing seeds and treating the host plant so as to reduce long chain-triacylglycerol production in the seeds under conditions for increasing acetyltriacylglycerol production in the seeds. In some embodiments, the treating comprises transfecting the host plant with a mutant gene whose expression is capable of reducing long chain-triacylglycerol production. In some embodiments, the mutant gene is a diacylglycerol acyltransferase 1 gene. In other embodiments, the treating is an inhibitory heterologous nucleic acid capable of interfering with the production of long chain triacylglycerol. In certain such embodiments, the inhibitory nucleic acid is selected from the group consisting of a diacylglycerol acyltransferase 1 gene, diacylglycerol acyltransferase 2 gene, and phospholipid acyltransferase gene. In some embodiments, the host plant comprises a heterologous nucleic acid sequence encoding a protein at least 95% identical to SEQ ID NO:1 or SEQ ID NO:2 and capable of forming an acetyltriacylglycerol. In some embodiments, the method further comprises step isolating the acetyltriacylglycerols from the seed. In some embodiments, the method further comprises step using the isolated acetyltriacylglycerols in an application selected from the group consisting of lubricant, biofuel, spray coating, food oil, in food processing, and thermoplastic polymer products. Additionally, in some embodiments, the inventions provide a seed produced by the methods. In some embodiments, the seed has low long chain-triacylglycerols. Additionally, in some embodiments, the inventions provide a composition comprising a seed produced by the methods. Additionally, in some embodiments, the inventions provide an oil isolated from the a seed produced by the methods. In some embodiments, the oil has low long chain-triacylglycerols. Additionally, in some embodiments, the inventions provide a composition comprising the oil. In some embodiments, the host plant comprises a heterologous nucleic acid sequence encoding a protein at least 95% identical to SEQ ID NO:1 or SEQ ID NO:2 and capable of forming an acetyltriacylglycerol.

In one or more embodiments, the present invention provides a method of operating a diesel engine. The method comprises combusting an oil produced in accordance with embodiments of the present invention in the diesel engine.

### BRIEF DESCRIPTION OF THE DRAWINGS

FIG. 1 is a drawing comparing TAG and acetyl-TAG, wherein acetyl-TAGs possess an sn-3 acetyl group that alters the physical and chemical properties of these molecules compared to regular TAGs;

FIGS. 2A and 2B are sequence alignments for various enzymes in accordance with embodiments of the present invention;

FIGS. 3A-3K are graphs showing ESI-MS analysis of lipid extracts from seeds of acetyl-TAG producing plants;

FIGS. 4A-4H are graphs showing yeast expressing EaDAcT homologs accumulate acetyl-TAG but not long chain TAG;

FIGS. 5A-5D show DAcT enzymes possessing in vitro and in vivo acetyl-TAG synthesis activity;

FIG. 6 shows a phylogenetic tree of plant MBOATs with different substrate specificities;

FIG. 7 shows multiple sequence alignment of plant MBOATs with different substrate specificities;

FIGS. 8A and 8B are images and graphs demonstrating identification of residues important for EaDAcT activity;

FIG. 9 is an image showing EaDAcT-D258E, EaDAcT-ΔV263 and EaDAcT-D258E-ΔV263 do not synthesize long chain TAG;

FIG. 10A is a graph showing in vitro acyltransferase activity of microsomes isolated from yeast expressing different acyltransferases, and FIG. 10B is a graph showing acetyl-TAGs levels in lipid extracts from yeast cells expressing different acyltransferases;

FIG. 11 is a scatter plot of the distribution of acetyl-TAGs, in T2 seed from 7 independent lines expressing EaDAcT and 23 independent lines expressing EfDAcT, wherein seeds without the visual DsRed transformation marker were excluded from analysis, horizontal lines represent the average value from each group, and asterisks indicate significant difference;

FIG. 12 is a scatter plot of acetyl-TAG levels from T2 transgenic Camelina sativa seed expressing EaDAcT+DGAT1 RNAi or EfDAcT+DGAT1 RNAi, wherein seeds without the visual DsRed transformation marker were excluded from analysis, and asterisks indicate significant difference;

FIG. 13A is a graph showing average fatty acid composition of T3 seed expressing EaDAcT+DGAT1 RNAi in 13 independent plant lines, and FIG. 13B is a graph showing average fatty acid composition of T₃ seed expressing EfDAcT+DGAT1 RNAi in 12 independent plant lines;

FIG. 14 is a graph showing viscosity of acetyl-TAG blends in accordance with embodiments of the present invention;

FIG. 15A is photograph showing standard diesel fuel combustion in a diesel engine, and FIG. 15B is a photograph showing acetyl-TAG combustion in a diesel engine in accordance with embodiments of the present invention; and

FIG. 16 is a graph showing combustion performance (pressure and heat release) for #2 diesel and acetyl-TAG for 300 kPa intake pressure in a diesel engine in accordance with embodiments of the present invention.

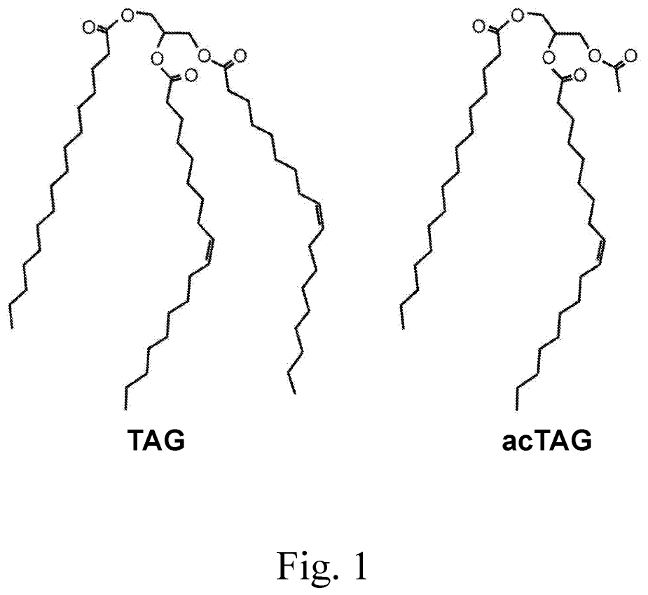

### DETAILED DESCRIPTION OF THE PREFERRED EMBODIMENT

In certain embodiments, the present invention generally relates to diacylglycerol acyltransferase genes and proteins having improved enzyme activity over existing technology, and methods of their use. In particular, the invention describes genes encoding proteins having increased diacylglycerol acetyltransferase activity, specifically for transferring an acetyl group to a diacylglycerol substrate to form acetyl-Triacylglycerols (acetyl-TAGS), for example, a 3-acetyl-1,2-diacyl-sn-glycerol, as compared to EaDAcT.

Described herein are acyltransferases from species other than Euonymus alatus that synthesize acetyl-TAGs. Specifically, two of these enzymes, EfDAcT and EkDAcT (derived from Euonymus fortunei and Euonymus kiautschovicus, respectively) possess unexpectedly higher enzyme activity than EaDAcT and other similar sequences. Importantly, however, enzymes from other species also unexpectedly lack acetyltransferase activity, when otherwise similar to EaDAcT. Therefore, in certain embodiments, the present invention is generally directed to EfDAcT and/or EkDAcT transferases, and their use in synthesizing acetyl-TAGs. In certain embodiments, the expression of these enzymes in transgenic organisms is capable of accumulating up to about 6-fold more acetyl-TAGs compared to EaDAcT and other similar sequences. In particularly preferred embodiments, EfDAcT and/or EkDAcT is expressed in transgenic Camelina sativa seeds, which is capable of producing unexpectedly higher levels of acetyl-TAGs compared to when EaDAcT is used.

In certain embodiments, the expression of EfDAcT and/or EkDAcT can be combined with the suppression of the competing DGAT1 enzyme. In certain such embodiments, acetyl-TAG levels as high as 90 mol % can be achieved in the best transgenic lines, which is an increase over previously published levels in the best EaDAcT lines. EfDAcT and EkDAcT therefore provide improved enzymes having increased activity compared to EaDAcT with regard to the production of acetyl-TAGs.

Oils produced from organisms expressing EfDAcT and/or EkDAcT exhibited similar viscosity and other properties as conventional diesel fuels. Accordingly, in certain embodiments, oils comprising acetyl-TAGS produced from organisms expressing EfDAcT and EkDAcT can be used as improved biofuel alternatives to conventional diesel and prior art biofuels.

Additional details regarding the enzymes and their specific uses are provided in the description below and in the Examples.

The present invention encompasses both native and recombinant wild-type forms of the transferases, as well as mutants and variant forms. The present invention also relates to methods of using the improved diacylglycerol acyltransferase genes and proteins, including their expression in transgenic organisms at commercially viable levels, for increasing production of 3-acetyl-1,2-diacyl-sn-glycerols in plant oils and altering the composition of oils produced by microorganisms, such as yeast, by increasing acetyl-TAG production. Additionally, oils produced by methods of the present inventions comprising genes and proteins are contemplated for use as biodiesel fuel, in polymer production, and as naturally produced food oils with reduced calories.

Several cultivated oil-seed crop plants contemplated for use (as biofuels and/or edible oils) with the genes and proteins of the present inventions are members of Brassicaceae (Cruciferae), also known as the mustard family and cabbage plant family. Further, the inventors contemplate isolating plant oils, including those described herein, from seeds of mustard and cabbage plants such as rapeseed (for example, Brassica sp., such as Brassica napus), Camelina (for example, Camelina sp. such as Camelina sativa), pennycress (Thlaspi arvense), mustard (for example, Brassica sp., such as Brassica alba, (for example, Crambe sp. such as Crambe abyssinica, also called sea cole, sea kale, Abyssinian mustard, Abyssinian kale, colewart, datran, etc.), field cabbage seed (Brassica sp. such as campestris, var. oleifera (for obtaining Colaz oil), Brassica campestris, var. chinensis—Bok choi, pak choi, pak choy, pok choi, etc.) and the like.

Further, the inventors contemplate the use of additional types of plants, including algae, for producing novel oils comprising acetyl-TAGS. Thus, the inventors contemplate inserting (transferring; transfecting) EfDAcT and/or EkDAcT into conventional oil-seed crop plants, including but not limited to an oil-seed crop of the Brassicea family.

Additionally, the inventors contemplate a system for large-scale production of oil comprising acetyl-TAGS for economically viable production. Thus, in some embodiments, plants currently grown for producing biofuel, oils, and food oil production are contemplated for use in combination with a heterologous acetyl-TAG producing gene of the present inventions, such as soybean plants, sunflower plants, rapeseed plants, flax plants, safflower plants, Jatropha plants, palm plants, Camelina plants, pennycress plants, Crambe plants, etc., and including plant families such as mustard family plants which include Arabidopsis plants (see, Examples), Camelina plants, and the like In one embodiment, a plant comprising an EfDAcT or EkDAcT gene further comprises a mutant DGAT1 gene, wherein the mutation reduces lc-TAG production. In other embodiments, the inventors contemplate inserting EfDAcT and/or EkDAcT into an oil crop plant comprising at least mutation resulting in the reduction of lc-TAGs compared to the wild-type plant. In one embodiment, the inventions provide a plant comprising an EfDAcT and/or EkDAcT gene of the present inventions whose seed contains Acetyl-TAGS greater than 50% (mol %), preferably greater than 80%, more preferably greater than 90%, and up to 99% of total TAGs. In one embodiment, a plant comprises an inhibitory nucleic acid capable of interfering with the production, wherein expression of the inhibitory nucleic acid reduced lc-TAG production. In certain such embodiments, the inhibitory nucleic acid is selected from the group consisting of a diacylglycerol acyltransferase 1 gene, diacylglycerol acyltransferase 2 gene, and phospholipid acyltransferase gene.

In one embodiment, the inventions provide a microorganism containing oil which is at least 40%, preferably at least 80%, more preferably at least 90%, and up to 100% acetyl-TAG (mol %).

Thus, in one embodiment, a microorganism comprising a EfDAcT or EkDAcT gene further comprises a mutant with inactivated TAG-synthesis genes, such as DGAT2 or PDAT or DGAT1, wherein the mutation reduces lc-TAG production. In a preferred embodiment, the microorganism is a yeast. In one embodiment, the yeast has mutations in a TAG-synthesis gene selected from the group consisting DGAT1 (such as a yeast ACAT-like ARE1 or ARE2 gene), in a preferred embodiment, the yeast has mutations in a TAG-synthesis gene selected from the group consisting of a DGAT2 and PDAT.

Even further, embodiments comprising compositions and methods of the present invention further relate to increasing the portion of acetyl-TAGS, such as 3-acetyl-1,2-diacyl-sn-glycerols, in plant oils isolated from plant parts, such as seeds, mesocarp, pericarp, stems, leaves, cells, including parts of algae, such as blades, intralamellar areas, surface areas, and oils released into culture media or enclosed systems, such as algal growth chambers and the like.

The present invention also relates to the efficient production of acetyl-triacylglycerols (acetyl-TAGS) by biological organisms (bio-production). In one embodiment, the acetyl-TAGS are for use in biofuels, in particular biodiesel as a fuel for engines. One exemplary use for a heterologous acetyl-TAG producing gene and encoded protein of the present inventions includes transforming a Camelina plant for producing acetyl-TAGS for use in combination with traditional diesel fuels and other types of biofuels. For example, a diesel fuel may be prepared by converting an acetyl-TAG oil of the present inventions isolated from a seed or plant part expressing a heterologous EfDAcT and/or EkDAcT gene encoding a polypeptide for inducing acetyl-TAG production of the present inventions and combining with other types of biofuels and/or traditional fuels. Thus, in some embodiments, the present invention includes a method of operating a diesel engine comprising combusting the oil of the present invention (including, for example, a fuel composition comprising the oil) in the diesel engine. A particularly preferred Camelina plant is Camelina sativa. Camelina plants have high productivity with limited rainfall and minimal soil fertility inputs, achieve higher seed yields compared to other Brassicaceae oil seed crops in drought conditions, have relatively short growing season (85-100 days), and possess winter and spring varieties. Traditional Camelina seed oil content ranges from 32-41% of seed weight, with the fatty acid profile being dominated by the polyunsaturated fatty acids linoleate (18:2) and linolenate (18:3).

Another exemplary use for a heterologous acetyl-TAG producing gene and encoded protein of the present invention includes transforming a Jatropha plant for producing acetyl-TAGs for use in combination with traditional jet fuels and other types of biofuels. Jatropha plants encompass a large grouping of mainly nonedible succulent plants, shrubs and trees that grow in nutrient poor soil. Specifically, Jatropha curcas (in addition to palm plants) are cultivated in plantations as feedstocks for transesterification to produce biodiesel. In addition to bio-diesel production, by-products of ‘Jatropha Curcas’ trans-esterification process are used to make a wide range of products including paper, energy pellets, soap, cosmetics, toothpaste, embalming fluid, pipe joint cement, cough medicine and as a moistening agent in tobacco.

In one embodiment, the inventors contemplate silencing (i.e. lowering) expression of lc-TAGS in host plants, by identifying plants with natural mutations, plants with induced mutations, and using plants or engineered mutant plants with lowered lc-TAG production as host cells for transfection or introgression of an EfDAcT or EkDAcT gene. The inventors further contemplated the use of such plants and plant cells for producing seeds with high amounts of acetyl-TAGS, either total yield per plant, i.e. total yield for acre or fraction of acetyl-TAG in the oil. Even further, the inventors contemplate the isolation of acetyl-TAGS from these seeds for use in biofuel or as novel oils for commercial uses. Additionally, the inventors contemplate the use of parental, T1, transgenic plants expressing EfDAcT and/or EkDAcT for use in breeding in order to develop commercially and/or agronomically viable cultivars and lines.

In particular, the present invention provides systems and methods for producing acetyl-TAGS with transgenic organisms expressing the diacylglycerol acyltransferase (DAcT) gene derived from EfDAcT and/or EkDAcT. In one embodiment a heterologous EfDAcT and/or EkDAcT gene is expressed in transgenic yeast cells, transgenic oil-seed crop plants and transgenic algae for providing novel oil. In a further embodiment, the oil is for use in providing biofuels. In other embodiments, EfDAcT and/or EkDAcT is expressed in transgenic yeast cells, transgenic oil-seed crop plants, transgenic algae, and transgenic fungi, where the oil recovered is used for providing novel TAG monomers for use in polymerization reactions, and even further embodiments for providing new types of polymers with commercial properties.

In another embodiment, the inventors contemplate a new polymer substrate. In particular, substitution of acetyl-TAGs for conventional TAGs in polymer production methods comprising polymer substrates will yield novel polymers with properties of conventional polymers. In another contemplated embodiment, acetyl-TAGS would provide relatively more linear polymers than conventional TAGs which would provide benefits to economic considerations of commercial polymer production. In a further embodiment, these novel TAG monomers would yield novel polymers with novel properties for commercial uses.

In yet another embodiment, organisms such as fungus, (for example, yeast) and plants producing the acetyl-TAGS of the present inventions are contemplated for use in methods for both edible oils and industrial oils, such that methods of acetyl-TAG oil production comprising one type of engineered organism expressing a nucleotide, protein, and oil of the present inventions would be used in the food industry and oleochemical industry and biofuels industry, as needed. Thus, for example, plant acreage designated for acetyl-TAG oil production using methods and compositions of the present inventions would produce an oil comprising acetyl-TAGS whose use would be designated after production for use in specific industries, such as the food industry, oleochemical industry, biofuels industry, etc. This contemplated post-production designation of use is in contrast to current practices of pre-production designated use currently necessary since each type of crop plant produces a limited type of oil for a narrow range of use. Thus, pre-production designation of acreage is necessary for meeting a particular type of oil need prior to seeding (planting) plants. Thus, unlike this current agriculture practice of designating acreage use, and thus the type of plants to cultivate, prior to planting, the use of plants of the present inventions would provide flexibility in oil production by eliminating pre planting acreage designation. Plants of the present inventions provide this flexibility since the oils comprising acetyl-TAGS of the present inventions would provide an oil feedstock capable of being used in a larger range of industries.

Additional contemplated benefits of using genes and proteins of improved diacylglycerol acyltransferase (DAcT) from Euonymus fortunei and Euonymus kauschovicus and other plants for producing novel oils include but are not limited to the following examples. Improved plants oils with large amounts of acetyl-TAGS would provide improved biofuels, such that acetyl-triacylglycerols in an improved oil-type would be directly extractable from oil-seed crops and provide improved properties for use in fuels. In preferred embodiments, these oils would need substantially less processing than current biodiesel feedstock oils or oils derived from prior art DAcT. Since these molecules have two instead of three long chain fatty acids, in one embodiment, esterification reaction times in methods comprising these oils for producing biodiesel would be significantly less than for TAG transesterification of three long chains. In a preferred embodiment, these improved oils comprising acetyl-TAGS would be used directly as fuel without esterification. In a further embodiment, oils comprising acetyl-TAGS of the present invention would be cold pressed (extracted) out of host cells (i.e. seeds, etc.) filtered than used directly as fuel by pouring the filtrate directly into a gas tank, oven fuel tank, fuel tank, or for direct use as a lubricant. In another embodiment, oils of the present inventions are blended with conventional biodiesel, such that methyl or ethyl esters would be blended with acetyl-TAGs produced by nucleic acids of the present inventions.

In another embodiment, the inventors contemplate a higher production capacity of acetyl-TAG per seed weight and thus a greater production capacity per hectare over currently available plant or algal oils comprising acetyl-TAGS. In particular, the inventors contemplate that transgenic expression of enzymes of the present inventions in oil-seed crops, such as soybean, sunflower, rapeseed (Canola), etc., acetyl-TAGS will be produced in relative abundance over levels of acetyl-TAGS in wild-type oils of these crops.

In another embodiment, the inventors contemplate lower production cost of biodiesel. Specifically, production of acetyl-TAGS in oils of plants for direct consumption would substantially lower costs associated with converting conventional TAGs to biofuels. In a preferred embodiment, the use of these improved oils would eliminate the necessity of chemical transesterification steps.

The present invention relates to compositions comprising diacylglycerol acyltransferase genes and polypeptides, and in particular Euonymus fortunei and Euonymus kiautschovicus diacylglycerol acyltransferase genes and polypeptides, where the enzyme exhibits primary specificity for diacylglycerol (DAG) and acyl-CoA substrates. These polypeptides are referred to as diacylglycerol acetyltransferases, designated ‘DAcT,’ indicating an activity of increased specificity for transfer of acetyl or related groups to DAG substrates, and/or ‘EfDAcT,’ and/or ‘EkDAcT,’ indicating an enzyme polypeptide obtained from or derived from Euonymus fortunei and Euonymus kauschovicus plants.

The present invention encompasses compositions comprising both native and recombinant forms of the enzymes, as well as mutant and variant forms, some of which possess altered characteristics relative to the wild-type. The present invention also comprises isolated lipids and isolated fatty acids from host cells expressing EfDAcT and/or EkDAcT genes and polypeptides. The present invention also comprises novel triacylglycerols synthesized by EfDAcT and/or EkDAcT. The present invention also provides methods for using EfDAcT and/or EkDAcT genes and polypeptides. The present invention also provides methods for isolating novel triacylglycerols synthesized by EfDAcT and/or EkDAcT.

In some embodiments, the present invention provides novel isolated nucleic acid sequences encoding EfDAcT and/or EkDAcT polypeptides. In other embodiments, the invention provides isolated nucleic acid sequences encoding mutants, variants, homologs, chimeras, and fusions of EfDAcT and/or EkDAcT polypeptides. In other embodiments, the present invention provides methods of generating such sequences. In other embodiments, the present invention provides methods of cloning and expressing such sequences, as well as methods of purifying and assaying the expression product of such sequences.

In additional embodiments, the present invention provides purified EfDAcT and/or EkDAcT genes, and EfDAcT and/or EkDAcT polypeptides. In some embodiments, the present invention provides purified EfDAcT and/or EkDAcT-like genes and EfDAcT and/or EkDAcT-like polypeptides from plants that have the capability to make acetyl-TAGS. Exemplary plants for providing EfDAcT and/or EkDAcT-like polypeptides for use in the present inventions include Celastraceae, exemplary plants include: Euonymus sp., (such as Euonymus europaeus, Euonymus latifolius, etc.), Celastrus sp., (such as Celastrus orbiculatus (Asiatic bittersweet), Celastrus scandens (American bittersweet)); Lardizabalaceae, exemplary plants include: Akebia quinata, Decaisnea fargesii, Lardizabala, biternata; Maytenus sp., (such as Maytenus ilicifolia, etc.)); Gymnosporia sp., (such as Gymnosporia harveyana, Gymnosporia Montana, Gymnosporia royleana, etc.); Ranunculaceae, exemplary plants include Adonis aestivalis, etc.), Rosaceae, exemplary plants include: Sorbus aucuparia, Sorbus mougeotii, other types of Mountain ashes, apple trees, peach trees, plum trees, strawberry plants; and the like. In other embodiments, the present invention provides mutants, variants, homologs, chimeras, and fusion proteins of EfDAcT and/or EkDAcT, and EfDAcT and/or EkDAcT-like (i.e. homologs and paralogs, etc.). In some embodiments, the present invention provides methods of purifying, and assaying the biochemical activity of wild type as well as mutants, variants, homologs, chimeras, and fusions of EfDAcT and/or EkDAcT, as well as methods of generating antibodies to such proteins.

In other embodiments, the present invention provides compositions comprising novel triacylglycerols synthesized by EfDAcT and/or EkDAcT polypeptides and/or EfDAcT and/or EkDAcT-like polypeptides from plants of the present invention. Such syntheses may be accomplished by any of the methods described below.

In some embodiments, the present invention provides methods of using novel isolated nucleic acid sequences encoding EfDAcT and/or EkDAcT polypeptides and/or -like polypeptides from plants to produce products of the acetyltransferase activity. In some embodiments, the methods involve adding the DAcT sequences to in vitro transcription and translation systems that include the substrates of the EfDAcT and EkDAcT polypeptides and/or DAcT polypeptides from other plants, such that the products of the acetyltransferase (oils) may be recovered (isolated). In other embodiments, the methods involve transforming organisms with DAcT sequences such that the sequences are expressed as products, such as EfDAcT and EkDAcT polypeptides and/or DAcT polypeptides from other plants. In particular embodiments, the products are recovered. In particular embodiments, the products are isolated. In other embodiments, the products remain in situ.

In some embodiments, the present invention provides methods of using recombinant EfDAcT and/or EkDAcT polypeptides and/or -like polypeptides from other plants (i.e. homologs, paralogs, etc.) to produce lipids containing acetyl or short-chain acyl groups as a result of the acetyltransferase and acyltransferase activity. In some embodiments, the methods involve adding the polypeptides to an in vitro system that includes the substrates of the DAcT (DAGs), such that the products of the DAcT may be recovered (isolated).

In other embodiments, the methods involve transforming a plant with a novel isolated nucleic acid sequence encoding EfDAcT and/or EkDAcT polypeptides and/or -like polypeptides from other plants, such that products of the DAcT are produced.

In some embodiments, the present invention provides an organism transformed with heterologous gene encoding an EfDAcT and/or EkDAcT polypeptide and/or -like polypeptides from other plants. In some embodiments, the organism is a microorganism. In some embodiments, the organism is a yeast cell. In some embodiments, the organism is an algal cell. In other embodiments, the organism is a nonalgal plant. In other embodiments, the organism is a plant part. In some embodiments, the present invention also provides a cell transformed with a heterologous gene encoding EfDAcT and/or EkDAcT polypeptides and/or -like polypeptides from plants. In some embodiments, the cell is a microorganism. In other embodiments, the cell is a plant cell.

In other embodiments, the present invention provides a plant seed transformed with a nucleic acid sequence encoding EfDAcT and/or EkDAcT polypeptides and/or -like polypeptides from plants. In yet other embodiments, the present invention provides an oil from a plant, a plant seed, or a microorganism transformed with a heterologous gene encoding an EfDAcT and/or EkDAcT polypeptide and/or -like polypeptides from plants.

The present invention provides compositions comprising purified EfDAcT and/or EkDAcT polypeptides as well as compositions comprising variants of EfDAcT and/or EkDAcT, including homologs, mutants, fragments, and fusion proteins thereof (as described further below).

In some embodiments of the present invention, the polypeptide is a purified product, obtained from expression of a native gene in a cell, while in other embodiments it may be a product of chemical synthetic procedures, and in still other embodiments it may be produced by recombinant techniques using a prokaryotic or eukaryotic host (for example, by bacterial, yeast, higher plant, insect and mammalian cells in culture). In some embodiments, depending upon the host employed in a recombinant production procedure, the polypeptide of the present invention may be glycosylated or may be non-glycosylated, or exhibit other post-translational amino acid modifications such as phosphorylation. In other embodiments, the polypeptides of the invention may also include an initial methionine amino acid residue.

In some embodiments, the polypeptide comprises a Euonymus DGAT; in other embodiments, the polypeptide comprises a Euonymus fortunei and Euonymus kauschovicus DGAT. In one embodiment, the polypeptide (SEQ ID NO:1 or SEQ ID NO:2) is encoded by an exemplary nucleic acid sequence (SEQ ID NO:18 or SEQ ID NO:19).

In other embodiments, the present invention provides isolated variants of the disclosed EfDAcT and/or EkDAcT polypeptides; these variants include mutants, fragments, fusion proteins or functional equivalents of EfDAcT and/or EkDAcT. In some embodiments, isolated variants include post-translational variants.

In some embodiments of the present invention, an EfDAcT and/or EkDAcT polypeptide purified from organisms is provided; such organisms include transgenic organisms, comprising a heterologous EfDAcT and/or EkDAcT gene, as well as organisms in which EfDAcT and/or

EkDAcT occurs naturally. In other embodiments, a EfDAcT and/or EkDAcT polypeptide is purified from an in vitro nucleic acid expression system, which comprises a nucleic acid sequence having a coding sequence of the present invention (for example, encoding a EfDAcT and/or EkDAcT, as, for example, SEQ ID NO:1 or SEQ ID NO:2 and genes encoding proteins at least 95% identical to SEQ ID NO:1 or SEQ ID NO:2) and from which the expressed EfDAcT and/or EkDAcT molecule can be purified (i.e., retaining the functional characteristics thereof). The present invention provides a purified EfDAcT and/or EkDAcT polypeptide as well as variants, including homologs, mutants, fragments, and fusion proteins thereof (as described further below).

The present invention also provides methods for recovering and purifying plant EfDAcT and/or EkDAcT from an organism or from an in vitro nucleic acid expression system; exemplary organisms include single and multi-cellular organisms. When isolated from an organism, the cells are typically first disrupted and then fractionated before subsequent enzyme purification.

Purification methods are also well-known, and include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, and isoelectric focusing. It is contemplated that EfDAcT and/or EkDAcT purified in an active or inactive form will require the presence of detergents to maintain its solubility in aqueous media during fractionation. It is further contemplated that assay of the enzyme activity will require removal of the detergent and reconstitution in liposomes to recover full activity.

The present invention further provides nucleic acid sequences having a coding sequence of the present invention (for example, SEQ ID NO:1 or SEQ ID NO:2) fused in frame to a marker sequence that allows for expression alone or both expression and purification of the polypeptide of the present invention. A non-limiting example of a marker sequence is a hexahistidine tag that may be supplied by a vector, for example, a pQE-30 vector which adds a hexahistidine tag to the N terminal of a EfDAcT and/or EkDAcT and which results in expression of the polypeptide in the case of a bacterial host, and in other embodiments by vector PT-23B, which adds a hexahistidine tag to the C terminal of a DAcT and which results in improved ease of purification of the polypeptide fused to the marker in the case of a bacterial host, or, for example, the marker sequence may be a hemagglutinin (HA) tag when a mammalian host is used. The HA tag corresponds to an epitope derived from the influenza hemagglutinin protein.

In some embodiments of the present invention, a EfDAcT and/or EkDAcT protein is produced using chemical methods to synthesize either an entire EfDAcT and/or EkDAcT amino acid sequence or a portion thereof. For example, peptides are synthesized by solid phase techniques, cleaved from the resin, and purified by preparative high performance liquid chromatography. In other embodiments of the present invention, the composition of the synthetic peptides is confirmed by amino acid analysis or sequencing.

Direct peptide synthesis can be performed using various solid-phase techniques and automated synthesis may be achieved. Additionally, an amino acid sequence of a EfDAcT and/or EkDAcT, or any part thereof, may be altered during direct synthesis and/or combined using chemical methods with other sequences to produce a variant polypeptide.

In some embodiments of the present invention, antibodies are generated to allow for the detection and characterization of a EfDAcT and/or EkDAcT protein. The antibodies may be prepared using various immunogens. In one embodiment, the immunogen is a EfDAcT and/or EkDAcT polypeptide (for example, an amino acid sequence as depicted in SEQ ID NO:1 or SEQ ID NO:2) or peptide fragments thereof or synthetic peptide fragment thereof, to generate antibodies that recognize EfDAcT and/or EkDAcT. Such antibodies include, but are not limited to polyclonal, monoclonal, chimeric, single chain, Fab fragments, and Fab expression libraries.

Various procedures known in the art may be used for the production of polyclonal antibodies directed against a EfDAcT and/or EkDAcT protein. For the production of antibody, various host animals can be immunized by injection with the peptide corresponding to a EfDAcT and/or EkDAcT epitope including but not limited to rabbits, mice, rats, sheep, goats, etc. In a preferred embodiment, the peptide is conjugated to an immunogenic carrier (for example, diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (for example, aluminum hydroxide), surface-active substances (for example, lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward a EfDAcT and/or EkDAcT, it is contemplated that any technique that provides for the production of antibody molecules by continuous cell lines in culture finds use with the present invention. These include but are not limited to the hybridoma technique and the EBV-hybridoma technique to produce human monoclonal antibodies.

In addition, it is contemplated that techniques described for the production of single chain antibodies find use in producing a EfDAcT and/or EkDAcT-specific single chain antibodies. An additional embodiment of the invention utilizes the techniques described for the construction of Fab expression libraries to allow rapid and easy identification of monoclonal F.sub.ab fragments with the desired specificity for a DAcT.

It is contemplated that any technique suitable for producing antibody fragments finds use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody is accomplished by techniques known in the art (for example, radioimmunoassay, ELISA (enzyme-linked immunosorbant assay), “sandwich” immunoassays, immunoradiometric assays, gel diffusion precipitin reactions, immunodiffusion assays, in situ immunoassays (for example, using colloidal gold, enzyme or radioisotope labels, for example), Western blots, precipitation reactions, agglutination assays (for example, gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay.

In some embodiments of the present invention, the foregoing antibodies are used in methods known in the art relating to the expression of a EfDAcT and/or EkDAcT (for example, for Western blotting), measuring levels thereof in appropriate biological samples, etc. The antibodies can be used to detect EfDAcT and/or EkDAcT in a biological sample from a plant. The biological sample can be an extract of a tissue, or a sample fixed for microscopic examination.

The biological samples are then tested directly for the presence of EfDAcT and/or EkDAcT using an appropriate strategy (for example, ELISA or radioimmunoassay) and format (for example, microwells, dipstick, etc. Alternatively, proteins in the sample can be size separated (for example, by polyacrylamide gel electrophoresis (PAGE), in the presence or not of sodium dodecyl sulfate (SDS), and the presence of EfDAcT and/or EkDAcT detected by immunoblotting (Western blotting). Immunoblotting techniques are generally more effective with antibodies generated against a peptide corresponding to an epitope of a protein, and hence, are particularly suited to the present invention.

The present invention provides compositions comprising purified nucleic acid sequences EfDAcT and/or EkDAcT. Coding sequences include but are not limited to genes, cDNA, and RNA. Thus, the present invention provides compositions comprising purified nucleic acid sequences encoding EfDAcT and/or EkDAcT, as well as nucleic acid sequences encoding variants of EfDAcT and/or EkDAcT, including homologs, mutants, or fragments, or fusion proteins thereof, as described above and below. In yet other embodiments, the nucleic acid sequences encode a portion of EfDAcT and/or EkDAcT that retains some functional characteristic of a DGAT. Examples of functional characteristics include the ability to act as an immunogen to produce an antibody that recognizes a DGAT.

Coding sequences for EfDAcT and/or EkDAcT include sequences isolated from an organism, which either comprises the coding sequence naturally or is transgenic and comprises a heterologous EfDAcT and/or EkDAcT coding sequence, sequences which are chemically synthesized and which may be codon-optimized, as well as sequences which represent a combination of isolated and synthesized (as, for example, where isolated sequences are mutagenized, or where a sequence comprises parts of sequences isolated from different sources and/or synthesized from different sources).

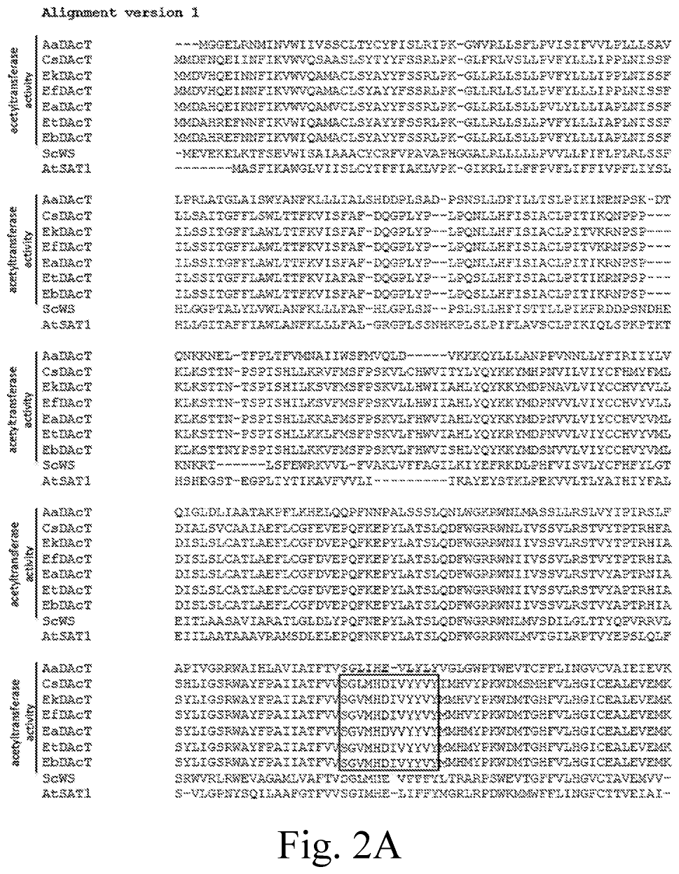

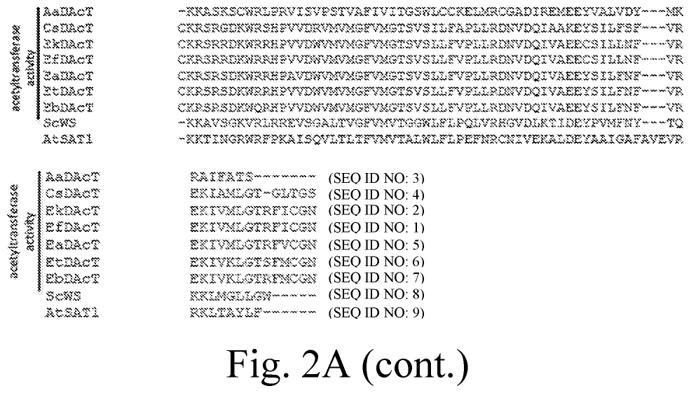

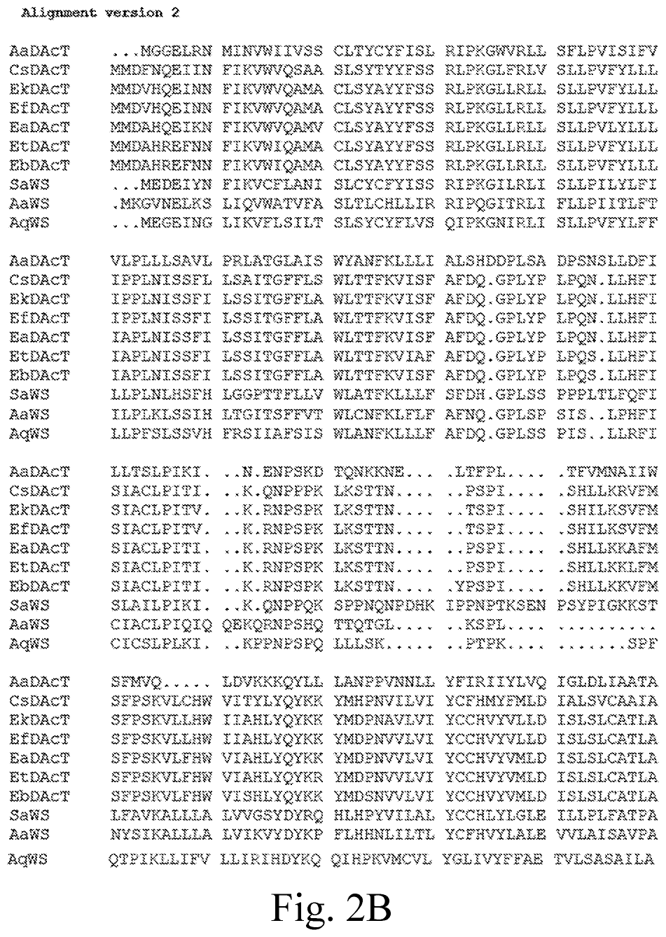

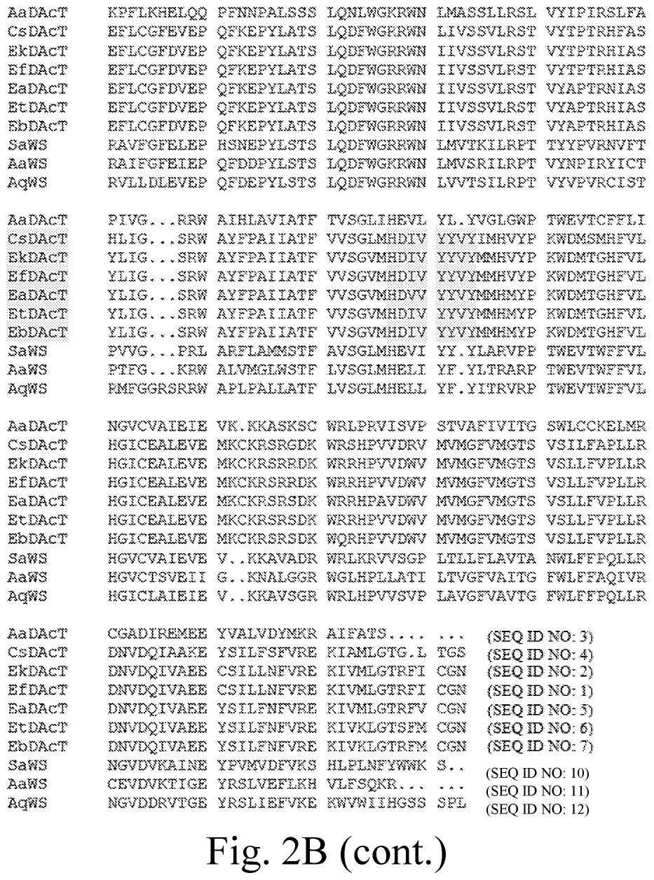

Thus, in some embodiments of the invention, the coding sequence of EfDAcT and/or EkDAcT is synthesized, whole or in part, using chemical methods well known in the art. In some embodiments, the sequences encode EfDAcT and/or EkDAcT. In some embodiments, the sequences comprise SEQ ID NO:1 or SEQ ID NO:2 shown in FIGS. 2A and 2B.

In other embodiments, the sequences encode a variant of the disclosed EfDAcT and/or EkDAcT polypeptides; these variants include mutants, fragments, fusion proteins or functional equivalents of EfDAcT and/or EkDAcT.

The present invention provides isolated nucleic acid sequences encoding EfDAcT and/or EkDAcT in addition to those described above. For example, some embodiments of the present invention provide isolated polynucleotide sequences that are capable of hybridizing to SEQ ID NO:18 or SEQ ID NO:19 under conditions of low to high stringency as long as the polynucleotide sequence capable of hybridizing encodes a protein that retains a desired biological activity of EfDAcT and/or EkDAcT. In preferred embodiments, hybridization conditions are based on the melting temperature (Tm) of the nucleic acid binding complex and confer a defined “stringency” as explained above.

In other embodiments of the present invention, alleles of a EfDAcT and/or EkDAcT are provided. In preferred embodiments, alleles result from a mutation, (in other words, a change in the nucleic acid sequence) and generally produce altered mRNAs or polypeptides whose structure or function may or may not be altered. Any given gene may have none, one or many allelic forms. Common mutational changes that give rise to alleles are generally ascribed to deletions, additions or substitutions of nucleic acids. Each of these types of changes may occur alone, or in combination with the others, and at the rate of one or more times in a given sequence.

Other embodiments of the present invention provide methods to isolate nucleic acid sequences encoding EfDAcT and/or EkDAcT. In some embodiments, the methods include the step of providing plant tissue in which acetyl-TAGS are present; this step is based upon the hypothesis that the presence of acetyl-TAGS in plant tissue, preferably seed tissue, is indicative of the presence of DGAT with diacylglycerol acetyltransferase activity, or a DAcT. Acetyl-TAG is present in a tissue if it is present at greater than about 1% of the total TAGs in that tissue; in preferred embodiments, acetyl-TAGS are present at greater than about 5% of the total TAGs in that tissue, or present at greater than about 10% of the total TAGs in that tissue.

In some embodiments, method involve obtaining a cDNA for EfDAcT and/or EkDAcT by using RT-PCR with degenerate primers to give a partial length clone, and subsequently using 3′ and 5′ RACE to define the 3′ and 5′ cDNA ends. A full length cDNA clone is then obtained via RT-PCR using primers based on the sequence of the 3′ and 5′ RACE products; this clone is then used to confirm the identity of the encoded polypeptide as EfDAcT and/or EkDAcT. Confirmation of the identity of the encoded polypeptide includes expressing the polypeptide of the sequence encoding a putative EfDAcT and/or EkDAcT (for example the full length cDNA clone), and characterizing the polypeptide of the putative DAcT coding sequence.

Characterization includes but is not limited to detecting the presence of the expressed polypeptide by antibody-binding (where, for example, the antibody is specific for EfDAcT and/or EkDAcT) or by detecting the reaction products of the expressed polypeptide. In further embodiments, acetyl-TAGS are present in the tissue from which the cDNA is prepared.

In yet other embodiments, methods involve first an examination of a plant expressed sequence tag (EST) database, in order to discover novel potential DGAT encoding sequences. Preferably, the plant source of the EST database comprises tissue in which acetyl-TAGS are present, such as its seed tissue. In some embodiments, examination of a plant EST database involves blasting the database with the amino acid sequence of the EfDAcT and/or EkDAcT in order to discover ESTs encoding amino acid sequences with homology to the EfDAcT and/or EkDAcT protein. In some further embodiments, the methods involve next assembling a clone encoding a complete putative DAcT and characterizing the expression products of such sequences so discovered as described above. In other further embodiments, these methods next involve sequencing likely candidate sequences, and characterizing the expression products of such sequences so discovered as described above. In some embodiments, EfDAcT and/or EkDAcT coding sequences, discovered by the methods of the present invention, can also be used to identify and isolate other plant genes. To isolate a gene, a ³²P-radiolabeled DAcT coding sequence (or cDNA) is used to screen, by DNA-DNA hybridization, a genomic library constructed from a plant genomic DNA. In further embodiments, acetyl-TAGS are present in the tissue from which the cDNA is prepared. Single isolated clones that test positive for hybridization are proposed to contain part or all of a EfDAcT and/or EkDAcT gene, and are sequenced. The sequence of the positive cloned plant genomic DNA is used to confirm the identity of the gene as EfDAcT and/or EkDAcT. If a particular clone encodes only part of the gene, additional clones that test positive for hybridization to the EfDAcT and/or EkDAcT coding sequence (or cDNA) are isolated and sequenced. Comparison of the full-length sequence of a putative DAcT gene to a cDNA is used to determine the location of introns, if they are present.

In other embodiments of the present invention, upstream sequences such as promoters and regulatory elements of a gene encoding a DAcT are detected by extending the gene by utilizing a nucleotide sequence encoding EfDAcT and/or EkDAcT in various methods known in the art. In some embodiments, it is contemplated that polymerase chain reaction (PCR) finds use in the present invention. This is a direct method that uses universal primers to retrieve unknown sequence adjacent to a known locus. First, genomic DNA is amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

In another embodiment, inverse PCR is used to amplify or extend sequences using divergent primers based on a known region. The primers may be designed using Oligo 4.0 (National Biosciences Inc, Plymouth Minn.), or another appropriate program, to be, for example, 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template. In yet other embodiments of the present invention, capture PCR is used. This is a method for PCR amplification of DNA fragments adjacent to a known sequence in human and yeast artificial chromosome (YAC) DNA. Capture PCR also requires multiple restriction enzyme digestions and ligations to place an engineered double-stranded sequence into an unknown portion of the DNA molecule before PCR. In still other embodiments, walking PCR is utilized. Walking PCR is a method for targeted gene walking that permits retrieval of unknown sequence. The PROMOTERFINDER kit (Clontech) uses PCR, nested primers and special libraries to “walk in” genomic DNA.

This process avoids the need to screen libraries and is useful in finding intron/exon junctions. In yet other embodiments of the present invention, add TAIL PCR is used as a preferred method for obtaining flanking genomic regions, including regulatory regions.

Preferred libraries for screening for full-length cDNAs include libraries that have been size-selected to include larger cDNAs. Also, random primed libraries are preferred, in that they contain more sequences that contain the 5′ and upstream gene regions. A randomly primed library may be particularly useful in cases where an oligo d(T) library does not yield full-length cDNA. Genomic libraries are useful for obtaining introns and extending 5′ sequence.

In some embodiments, the present invention provides isolated variants of the disclosed nucleic acid sequence encoding EfDAcT and/or EkDAcT, and the polypeptides encoded thereby; these variants include mutants, fragments, fusion proteins, or functional equivalents of EfDAcT and/or EkDAcT. Thus, nucleotide sequences of the present invention are engineered in order to alter EfDAcT and/or EkDAcT coding sequence for a variety of reasons, including but not limited to alterations that modify the cloning, processing and/or expression of the gene product (such alterations include inserting new restriction sites, altering glycosylation patterns, and changing codon preference) as well as varying the enzymatic activity (such changes include but are not limited to differing substrate affinities, differing substrate preferences and utilization, differing inhibitor affinities or effectiveness, differing reaction kinetics, varying subcellular localization, and varying protein processing and/or stability). For example, mutations are introduced which alter the substrate specificity, such that the preferred substrate is changed.

Some embodiments of the present invention provide mutant forms of EfDAcT and/or EkDAcT (in other words, muteins). In preferred embodiments, variants result from mutation, (in other words, a change in the nucleic acid sequence) and generally produce altered mRNAs or polypeptides whose structure or function may or may not be altered. Any given gene may have none, one, or many mutant forms. Common mutational changes that give rise to variants are generally ascribed to deletions, additions or substitutions of nucleic acids. Each of these types of changes may occur alone, or in combination with the others, and at the rate of one or more times in a given sequence. Still other embodiments of the present invention provide isolated nucleic acid sequence encoding EfDAcT and/or EkDAcT homologs, and the polypeptides encoded thereby.

It is contemplated that is possible to modify the structure of a peptide having an activity (for example, a diacylglycerol acetyltransferase activity) for such purposes as increasing synthetic activity or altering the affinity of the EfDAcT and/or EkDAcT for a substrate, or for increasing stability or turnover or subcellular location of the polypeptide. Such modified peptides are considered functional equivalents of peptides having an activity of a EfDAcT and/or EkDAcT. A modified peptide can be produced in which the nucleotide sequence encoding the polypeptide has been altered, such as by substitution, deletion, or addition.

In some preferred embodiments of the present invention, the alteration increases synthetic activity or alters the affinity of the EfDAcT and/or EkDAcT for a particular acetyl- or related group-CoA or acetyl or related group acceptor substrate. In particularly preferred embodiments, these modifications do not significantly reduce the synthetic activity of the modified enzyme. In other words, construct “X” can be evaluated in order to determine whether it is a member of the genus of modified or variant EfDAcT and/or EkDAcT of the present invention as defined functionally, rather than structurally. In some embodiments the present invention provides nucleic acids encoding a EfDAcT and/or EkDAcT that complement the coding region of SEQ ID NO:1 and/or SEQ ID NO:2. In other embodiments, the present invention provides nucleic acids encoding EfDAcT and/or EkDAcT that compete for the binding of diacylglycerol or acetyl substrates with the protein encoded by SEQ ID NO:1 and/or SEQ ID NO:2.

In other preferred embodiments of the alteration, the alteration results in intracellular half-lives dramatically different from that of the corresponding wild-type protein. For example, an altered protein is rendered either more stable or less stable to proteolytic degradation or other cellular process that result in destruction of, or otherwise inactivate EfDAcT and/or EkDAcT. Such homologs, and the genes that encode them, can be utilized to alter the activity of EfDAcT and/or EkDAcT by modulating the half-life of the protein. For instance, a short half-life can give rise to more transient EfDAcT and/or EkDAcT biological effects. Other variants have characteristics which are either similar to wild-type EfDAcT and/or EkDAcT, or which differ in one or more respects from wild-type EfDAcT and/or EkDAcT.

Mutant forms of EfDAcT and/or EkDAcT are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (in other words, conservative mutations) will not have a major effect on the biological activity of the resulting molecule. Accordingly, some embodiments of the present invention provide variants of EfDAcT and/or EkDAcT disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

More rarely, a variant includes “nonconservative” changes (for example, replacement of a glycine with a tryptophan). Analogous minor variations can also include amino acid deletions or insertions, or both. Guidance in determining which amino acid residues can be substituted, inserted, or deleted without abolishing biological activity can be found using computer programs (for example, LASERGENE software, DNASTAR Inc., Madison, Wis.).

Mutants of EfDAcT and/or EkDAcT can be generated by any suitable method well known in the art, including but not limited to site-directed mutagenesis, randomized “point” mutagenesis, and domain-swap mutagenesis in which portions of the Euonymus DGAT cDNA are “swapped” with the analogous portion of other plant or bacterial DGAT-encoding cDNAs.

Variants may be produced by methods such as directed evolution or other techniques for producing combinatorial libraries of variants. Thus, the present invention further contemplates a method of generating sets of combinatorial mutants of the present EfDAcT and/or EkDAcT proteins, as well as truncation mutants, and is especially useful for identifying potential variant sequences (in other words, homologs) that possess the biological activity of EfDAcT and/or EkDAcT (for example, transfer of an acetyl or related group to diacylglycerol). In addition, screening such combinatorial libraries is used to generate, for example, novel EfDAcT and/or EkDAcT homologs that possess novel substrate specificities or other biological activities; examples of substrate specificities are described above.

It is contemplated that the EfDAcT and/or EkDAcT nucleic acids can be utilized as starting nucleic acids for directed evolution. These techniques can be utilized to develop EfDAcT and/or EkDAcT variants having desirable properties such as increased synthetic activity or altered affinity for a particular acyl-CoA or acyl acceptor substrate.

In some embodiments, artificial evolution is performed by random mutagenesis (for example, by utilizing error-prone PCR to introduce random mutations into a given coding sequence). This method requires that the frequency of mutation be finely tuned.

In other embodiments of the present invention, the polynucleotides of the present invention are used in gene shuffling or sexual PCR procedures. Gene shuffling involves random fragmentation of several mutant DNAs followed by their reassembly by PCR into full-length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes. Variants produced by directed evolution can be screened for DGAT activity.

In addition, the present invention provides isolated nucleic acid sequences encoding fragments of EfDAcT and/or EkDAcT (in other words, truncation mutants), and the polypeptides encoded by such nucleic acid sequences. In preferred embodiments, the EfDAcT and/or EkDAcT fragment is biologically active.

In some embodiments of the present invention, when expression of a portion of a EfDAcT and/or EkDAcT protein is desired, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). Removal of an N-terminal methionine, if desired, can be achieved either in vivo by expressing such recombinant polypeptides in a host that produces MAP (for example, E. coli or CM89 or S. cerevisiae), or in vitro by use of purified MAP.

The present invention also provides nucleic acid sequences encoding fusion proteins incorporating all or part of EfDAcT and/or EkDAcT, and the polypeptides encoded by such nucleic acid sequences. In some embodiments, the fusion proteins have a EfDAcT and/or EkDAcT functional domain with a fusion partner. Accordingly, in some embodiments of the present invention, the coding sequences for the polypeptide (for example, a EfDAcT and/or EkDAcT functional domain) is incorporated as a part of a fusion gene including a nucleotide sequence encoding a different polypeptide. In one embodiment, a single fusion product polypeptide transfers an acetyl group to diacylglycerol (one fusion partner possesses the ability to synthesize acetyl-TAG).

In some embodiments of the present invention, chimeric constructs code for fusion proteins containing a portion of EfDAcT and/or EkDAcT and a portion of another gene. In some embodiments, the fusion proteins have biological activity similar to the wild type EfDAcT and/or EkDAcT (for example, have at least one desired biological activity of EfDAcT and/or EkDAcT). In other embodiments, the fusion proteins have altered biological activity.

In other embodiments of the present invention, chimeric constructs code for fusion proteins comprising EfDAcT and/or EkDAcT gene or portion thereof and a leader or other signal sequences which direct the protein to targeted subcellular locations. Such sequences are well known in the art and direct proteins to locations such as the chloroplast, the mitochondria, the endoplasmic reticulum, the tonoplast, the Golgi network, and the plasmalemma.

In addition to utilizing fusion proteins to alter biological activity, it is widely appreciated that fusion proteins can also facilitate the expression and/or purification of proteins, such as a EfDAcT and/or EkDAcT protein. Accordingly, in some embodiments of the present invention, EfDAcT and/or EkDAcT is generated as a glutathione-S-transferase (in other words, GST fusion protein). It is contemplated that such GST fusion proteins enables easy purification of EfDAcT and/or EkDAcT, such as by the use of glutathione-derivatized matrices.

In another embodiment of the present invention, a fusion gene coding for a purification leader sequence, such as a poly-(His)/enterokinase cleavage site sequence at the N-terminus of the desired portion of EfDAcT and/or EkDAcT allows purification of the expressed EfDAcT and/or EkDAcT fusion protein by affinity chromatography using a Ni.sup.2+ metal resin. In still another embodiment of the present invention, the purification leader sequence is then subsequently removed by treatment with enterokinase. In yet other embodiments of the present invention, a fusion gene coding for a purification sequence appended to either the N (amino) or the C (carboxy) terminus allows for affinity purification; one example is addition of a hexahistidine tag to the carboxy terminus of EfDAcT and/or EkDAcT, which is contemplated to be useful for affinity purification. In yet other embodiments of the present invention, epitope tags of EfDAcT and/or EkDAcT are prepared.

A wide range of techniques are known in the art for screening gene products of combinatorial libraries made by point mutations, and for screening cDNA libraries for gene products having a certain property. Such techniques are generally adaptable for rapid screening of the gene libraries generated by the combinatorial mutagenesis of EfDAcT and/or EkDAcT homologs. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

In light of the present disclosure, other forms of mutagenesis generally applicable will be apparent to those skilled in the art in addition to the aforementioned rational mutagenesis based on conserved versus non-conserved residues. For example, EfDAcT and/or EkDAcT homologs can be generated and screened using, for example, alanine scanning mutagenesis and the like.

In other embodiment of the present invention, nucleic acid sequences corresponding to the EfDAcT and/or EkDAcT genes, homologs and mutants as described above may be used to generate recombinant DNA molecules that direct the expression of the encoded protein product in appropriate host cells.

EfDAcT and/or EkDAcT-encoding nucleotide sequences possessing non-naturally occurring codons may also find use for producing novel oils. Therefore, in some preferred embodiments, codons preferred by a particular prokaryotic or eukaryotic host can be selected, for example, to increase the rate of DAcT expression or to produce recombinant RNA transcripts having desirable properties, such as a longer half-life, than transcripts produced from naturally occurring sequence.

The nucleic acid sequences of the present invention may be employed for producing polypeptides by recombinant techniques. Thus, for example, the nucleic acid sequence may be included in any one of a variety of expression vectors for expressing a polypeptide. In some embodiments of the present invention, vectors include, but are not limited to, chromosomal, nonchromosomal and synthetic DNA sequences (for example, derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising one or more of the nucleic acid sequences as broadly described above (for example, SEQ ID NO:1 or SEQ ID NO:2). In some embodiments of the present invention, the constructs comprise a vector, such as a plasmid or viral vector, into which a nucleic acid sequence of the invention has been inserted, in a forward or reverse orientation. In preferred embodiments of the present invention, the appropriate nucleic acid sequence is inserted into the vector using any of a variety of procedures. In general, the nucleic acid sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial constructs, such as pQE70, pQE60, pQE-9 (Qiagen), pBS, pD10, phagescript, psiX174, pBluescript SK, pBSKS, pNH8A, pNH16a, pNH18A, pNH46A (Stratagene); ptrc99a, pKK223-3, pKK233-3, pDR540, pRIT5 (Pharmacia); and 2) Eukaryotic—pWLNEO, pSV2CAT, pOG44, PXT1, pSG (Stratagene) pSVK3, pBPV, pMSG, and pSVL (Pharmacia). Any other plasmid or vector, including vectors for use with Agrobacterium expression systems, plant cell, plant seed expression, algal expression, fungal, i.e. yeast expression, may be used as long as they are replicable and viable in the host. In some preferred embodiments of the present invention, plant expression vectors comprise an origin of replication, a suitable promoter and enhancer, and also any necessary ribosome binding sites, polyadenylation sites, splice donor and acceptor sites, transcriptional termination sequences, and 5′ flanking nontranscribed sequences. In other embodiments, DNA sequences derived from the SV40 splice, and polyadenylation sites may be used to provide the required nontranscribed genetic elements.

In certain embodiments of the present invention, a nucleic acid sequence of the present invention within an expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda PL and PR, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (for example, dihydrofolate reductase or neomycin resistance for eukaryotic cell culture, or tetracycline or ampicillin resistance in E. coli).

In some embodiments of the present invention, transcription of the DNA encoding polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin by 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

In a further embodiment, the present invention provides host cells containing any of the above-described constructs. In some embodiments of the present invention, the host cell is a higher eukaryotic cell (for example, a plant cell). In other embodiments of the present invention, the host cell is a lower eukaryotic cell (for example, a yeast cell). In still other embodiments of the present invention, the host cell can be a prokaryotic cell (for example, a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, 293T, C127, 3T3, HeLa and BHK cell lines, NT-1 (tobacco cell culture line), root cell and cultured roots in rhizosecretion. Other examples include microspore-derived cultures of oilseed rape, and transformation of pollen and microspore culture systems.

The constructs in host cells can be used in a conventional manner to produce the gene product encoded by any of the recombinant sequences of the present invention described above. In some embodiments, introduction of the construct into the host cell can be accomplished by calcium phosphate transfection, DEAE-Dextran mediated transfection, or electroporation. Alternatively, in some embodiments of the present invention, a polypeptide of the invention can be synthetically produced by conventional peptide synthesizers.

Proteins can be expressed in eukaryotic cells, yeast, bacteria, or other cells under the control of appropriate promoters. Cell-free translation systems can also be employed to produce such proteins using RNAs derived from a DNA construct of the present invention.

In some embodiments of the present invention, following transformation of a suitable host strain and growth of the host strain to an appropriate cell density, the selected promoter is induced by appropriate means (for example, temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

In one aspect of the present invention, methods are provided for producing acetyl glycerides (acetyl-TAGS). Although the following methods are described in terms of production of acetyl-TAGS, it is understood that these methods are also applicable to a DAcT that transfers a related group, resulting in production of TAGs to which the group related to acetate is transferred. In some embodiments, acetyl-TAGS are produced in vivo, in organisms transformed with a heterologous gene encoding a polypeptide exhibiting diacylglycerol acetyltransferase activity and grown under conditions sufficient to effect production of acetyl-TAGS. In other embodiments, acetyl-TAGS are produced in vitro, from either nucleic acid sequences encoding EfDAcT and/or EkDAcT or from polypeptides exhibiting diacylglycerol acetyltransferase activity.

By controlling the type of substrate, it is possible to produce TAGs. It is further contemplated that the use of the EfDAcT and/or EkDAcT can be used to produce structures such as acetyldiricinolein; acetyldivernolin, or acetyldicaprin.

In some embodiments, compounds are produced by incubating a EfDAcT and/or EkDAcT enzyme with acetyl-CoA and the appropriate DAG substrate (for example, diricinolein or divernolin) under suitable conditions such that the acetyl-TAG products are synthesized. In other embodiments, compounds are produced by incubating a EfDAcT and/or EkDAcT enzyme with a DAG substrate and an appropriate related group-CoA (for example, cinnamoyl) under suitable conditions such that novel TAG products (for example, cinnamoyl-TAG, etc.) are synthesized. It is contemplated that cinnamoyl-TAG will absorb UV and can be used in sunscreens.

Such compounds can be produced in vivo by transforming a plant in which the appropriate DAG substrate is present with a gene encoding EfDAcT and/or EkDAcT under control of a suitable promoter, such that EfDAcT and/or EkDAcT is expressed when and where the appropriate DAG and acyl-CoA substrates are synthesized, resulting in the synthesis of a novel TAG. The DAG and acyl-CoA substrates may be endogenous substrates, or may be the products of expression of additional genes, including genes for biosynthetic enzymes or for up-regulating pathways.

In some embodiments of the present invention, acetyl-TAGS are produced in vivo, by providing an organism transformed with a heterologous gene encoding EfDAcT and/or EkDAcT and growing the transgenic organism under conditions sufficient to effect production of acetyl-TAGS. In other embodiments of the present invention, acetyl-TAGS are produced in vivo by transforming an organism with a heterologous gene encoding EfDAcT and/or EkDAcT and growing the transgenic organism under conditions sufficient to effect production of acetyl-TAGS.

Organisms which are transformed with a heterologous gene encoding EfDAcT and/or EkDAcT include preferably those which naturally synthesize and store in some manner triacylglycerols (TAGs), and those which are commercially feasible to grow and suitable for harvesting large amounts of the TAG products. Such organisms include but are not limited to, oleaginous yeast and algae, and plants and animals. Examples of yeasts include oleaginous yeast, which include but are not limited to the genera Lipomyces, Candida, Rhodotorula, Rhodosporidium and Cryptococcus, which can be grown in commercial-scale fermenters. Examples of plants include preferably oil-producing plants, such as soybean, rapeseed and canola, sunflower, cotton, corn, cocoa, safflower, oil palm, coconut palm, flax, castor, and peanut. Many commercial cultivars can be transformed with heterologous genes. In cases where that is not possible, non-commercial cultivars of plants can be transformed, and the trait for expression of EfDAcT and/or EkDAcT moved to commercial cultivars by breeding techniques well known in the art.

A heterologous gene encoding EfDAcT and/or EkDAcT, which includes variants of EfDAcT and/or EkDAcT, includes any suitable sequence of the invention as described above. Preferably, the heterologous gene is provided within an expression vector such that transformation with the vector results in expression of the polypeptide; suitable vectors are described above and following.

A transgenic organism is grown under conditions sufficient to effect production of acetyl-TAGS. In some embodiments of the present invention, a transgenic organism is supplied with exogenous substrates of the DAcT (for example, in a fermentor). Such substrates can comprise sugars as carbon sources for TAG synthesis, fatty acids and glycerol used directly for the production of DAG and TAG, DAG itself, and acetic acid which will both provide a general carbon source and be used for the production of acetyl-CoA and/or diacylglycerols (DAGs). When related groups are transferred to DAG, such substrates may instead or in addition be provided to the transgenic organism; exemplary related group include but are not limited to butyrate, propionate, and cinnamate. Substrates may be supplied in various forms as are well known in the art; such forms include aqueous suspensions prepared by sonication, aqueous suspensions prepared with detergents and other surfactants, dissolution of the substrate into a solvent, and dried powders of substrates. Such forms may be added to organisms or cultured cells or tissues grown in fermenters.

In yet other embodiments of the present invention, a transgenic organism comprises a heterologous gene encoding EfDAcT and/or EkDAcT operably linked to an inducible promoter, and is grown either in the presence of the an inducing agent, or is grown and then exposed to an inducing agent. In still other embodiments of the present invention, a transgenic organism comprises a heterologous gene encoding EfDAcT and/or EkDAcT operably linked to a promoter which is either tissue specific or developmentally specific, and is grown to the point at which the tissue is developed or the developmental stage at which the developmentally specific promoter is activated. Such promoters include seed specific promoters.

In alternative embodiments, a transgenic organism as described above is engineered to produce greater amounts of the diacylglycerol substrate. Thus, it is contemplated that a transgenic organism may include further modifications such that fatty acid synthesis is increased, and may in addition or instead include exogenous acyltransferases, phosphatidylcholine:diacylglycerol cholinephosphotransferase and/or phosphatidic acid phosphatases. In one exemplary embodiment, fatty acid synthesis is altered by producing nonfunctional FAE1 protein, i.e. truncated mutant FAE1 protein. In one exemplary embodiment, fatty acid synthesis is altered by reducing FAE1 protein production. In other embodiments of the present invention, a host organism produces large amounts of a desired substrate, such as acetyl-CoA or DAG; non-limiting examples include organisms transformed with genes encoding acetyl-CoA synthetases and/or ATP citrate lyase.

In some embodiments, it is contemplated that certain DAGs will result in the synthesis of novel acetyl-TAGS with desirable properties. Thus, a particularly suitable host is one that produces a high proportion of such a DAG. Such hosts may include organisms with high levels of oleic, ricinoleic or vernolic acids, or of short- and medium-chain fatty acids. These hosts may include plants such as Cuphea, Vernonia or Euphorbia species, which are undergoing domestication; plants such as Ricinus communis, which is a specialty oil crop; plants such as Brassica and soybean, for which high oleic lines have been developed; and transgenic plants where the endogenous fatty acid composition of the seed oil has been altered by seed-specific expression of biosynthetic genes.

In other embodiments, a host organism produces low amounts of endogenous TAGs but retain the capacity to up-regulate the synthesis of DAG when there is a draw on the DAG pool. It is contemplated that in such hosts, novel TAGs produced from an exogenous EfDAcT and/or EkDAcT are a higher proportion of the total TAGs; advantages include less expensive purification of the novel TAGs. Non-limiting exemplary hosts include those with low endogenous DGAT activity (either or both DGAT1 or DGAT2), PDAT activity or other acyltransferase activity resulting in the synthesis of TAGs. Such hosts may occur naturally or via genetic engineering techniques. Non-limiting exemplary techniques include knock-out produced by EMS and transposon tagging.

In other embodiments of the present invention, the methods for producing acetyl-TAGS further comprise collecting the acetyl-TAGS produced. Such methods are known generally in the art, and include harvesting the transgenic organisms and extracting the acetyl-TAGS.

Plants are transformed with at least a heterologous gene encoding EfDAcT and/or EkDAcT according to procedures well known in the art. It is contemplated that the heterologous gene is utilized to increase the level of the enzyme activities encoded by the heterologous gene.

The methods of the present invention are not limited to any particular plant. Indeed, a variety of plants are contemplated, including but not limited to tomato, potato, tobacco, pepper, rice, corn, barley, wheat, Brassica, Arabidopsis, sunflower, soybean, poplar, and pine. Preferred plants include oil-producing species, which are plant species that produce and store triacylglycerol in specific organs, primarily in seeds. Such species include but are not limited to soybean (Glycine max), rapeseed and canola (including Brassica napus and B. campestris), sunflower (Helianthus annus), cotton (Gossypium hirsutum), corn (Zea mays), cocoa (Theobroma cacao), safflower (Carthamus tinctorius), oil palm (Elaeis guineensis), coconut palm (Cocos nucifera), flax (Linum usitatissimum), castor (Ricinus communis) and peanut (Arachis hypogaea). The group also includes non-agronomic species which are useful in developing appropriate expression vectors such as tobacco, rapid cycling Brassica species, and Arabidopsis thaliana, and wild species undergoing domestication, such as Vernonia and Cuphea, which may be a source of unique fatty acids. In addition plant lines where the endogenous DGAT gene(s) has been inactivated by any method, but including mutagenesis, transposon tagging, hairpin RNA and chimeraplasty, are considered ideal for optimum when used in conjunction with expression of the Euonymus DAcT gene. In addition lines where DGAT genes from other gene families and other routes to TAG such as PDAT have been down regulated are contemplated. In addition plants engineered to make increased amounts of medium chain fatty acids (which are consequently incorporated into DAG and then into TAG) are contemplate for transformation with EfDAcT and/or EkDAcT to produce oil with further reductions in kinematic viscosity. Such plant engineering would be accomplished by methods comprising altering fatty acid synthesizing enzymes, such as acyl-ACP thioesterases, i.e. FATB enzymes and a FATB genetic engineering strategy. Different plant lines may have different seed oil fatty acid compositions, which may be generated by selection, mutagenesis or a genetic engineering strategy, and thus may furnish different products when transformed with EfDAcT and/or EkDAcT of this invention.

Additional types of natural and engineered plants are contemplated for use in the present inventions, such plants produce low levels of TAGs comprising short chain TAGs, medium chain TAGs and combinations thereof.

The methods of the present invention contemplate the use of at least a heterologous gene encoding EfDAcT and/or EkDAcT, as described above. Heterologous genes intended for expression in plants are first assembled in expression cassettes comprising a promoter. Methods which are well known to those skilled in the art may be used to construct expression vectors containing a heterologous gene and appropriate transcriptional and translational control elements. These methods include in vitro recombinant DNA techniques, synthetic techniques, and in vivo genetic recombination.

In general, these vectors comprise a nucleic acid sequence of the invention encoding EfDAcT and/or EkDAcT (as described above) operably linked to a promoter and other regulatory sequences (for example, enhancers, polyadenylation signals, etc.) required for expression in a plant.

Promoters include but are not limited to constitutive promoters, tissue-, organ-, and developmentally specific promoters, and inducible promoters. Examples of promoters include but are not limited to: constitutive promoter 35S of cauliflower mosaic virus; a wound-inducible promoter from tomato, leucine amino peptidase; a chemically-inducible promoter from tobacco, Pathogenesis-Related 1 (PR1) (induced by salicylic acid and BTH (benzothiadiazole-7-carbothioic acid 5-methyl ester)); a tomato proteinase inhibitor II promoter (PIN2) or LAP promoter (both inducible with methyl jasmonate); a heat shock promoter; a tetracycline-inducible promoter; seed-specific promoters, such as those for seed storage proteins (for example, phaseolin, napin, oleosin, and a promoter for soybean beta conglycin, and 25 promoter sequences, such as an Arabidopsis seed specific promoter, found on BAC T24A18, nucleotides 31032 to 32179, and promoters for lipid biosynthetic genes such as DGAT1 and FAE1.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its optimal polyadenylation. Appropriate transcriptional terminators and those which are known to function in plants include, but are not limited to, the CaMV 355 terminator, the tml terminator, the pea rbcS E9 terminator, and the nopaline and oetopine synthase terminator.

In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize Adh1 gene have been found to significantly enhance the expression of the wild-type gene under its cognate promoter when introduced into maize cells. Intron sequences have been routinely incorporated into plant transformation vectors, typically within the non-translated leader.

In some embodiments of the present invention, the construct for expression of the nucleic acid sequence of interest also includes a regulator such as a nuclear localization signal, a plant translational consensus sequence, an intron, and the like, operably linked to the nucleic acid sequence encoding EfDAcT and/or EkDAcT.

In preparing a construct comprising a nucleic acid sequence encoding EfDAcT and/or EkDAcT, various DNA fragments can be manipulated, so as to provide for the DNA sequences in the desired orientation (for example, sense or antisense) orientation and, as appropriate, in the desired reading frame. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like is preferably employed, where insertions, deletions or substitutions (for example, transitions and transversions) are involved.

Numerous transformation vectors are available for plant transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics, the bar gene which confers resistance to the herbicide phosphinothricin, the hph gene which confers resistance to the antibiotic hygromycin, and the dhfr gene, which confers resistance to methotrexate.

In some preferred embodiments, the vector is adapted for use in an Agrobacterium mediated transfection process. Construction of recombinant Ti and Ri plasmids in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

In other embodiments of the invention, the nucleic acid sequence of interest is targeted to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences. One of skill in the art knows that homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

In yet other embodiments, the nucleic acids of the present invention are utilized to construct vectors derived from plant (+) RNA viruses (for example, brome mosaic virus, tobacco mosaic virus, alfalfa mosaic virus, cucumber mosaic virus, tomato mosaic virus, and combinations and hybrids thereof). Generally, the inserted EfDAcT and/or EkDAcT polynucleotide of the present invention can be expressed from these vectors as a fusion protein (for example, coat protein fusion protein) or from its own subgenomic promoter or other promoter.

In some embodiments of the present invention the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator.

Once a nucleic acid sequence encoding EfDAcT and/or EkDAcT is operatively linked to an appropriate promoter and inserted into a suitable vector for the particular transformation technique utilized (for example, one of the vectors described above), the recombinant DNA described above can be introduced into the plant cell in a number of art-recognized ways. Those skilled in the art will appreciate that the choice of method might depend on the type of plant targeted for transformation. In some embodiments, the vector is maintained episomally. In other embodiments, the vector is integrated into the genome.

In some embodiments, direct transformation in the plastid genome is used to introduce the vector into the plant cell. The basic technique for chloroplast transformation involves introducing regions of cloned plastid DNA flanking a selectable marker together with the nucleic acid encoding the RNA sequences of interest into a suitable target tissue (for example, using biolistic techniques or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation. The presence of cloning sites between these markers allowed creation of a plastid targeting vector introduction of foreign DNA molecules. Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase. Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of the RNAs encoded by the DNA molecule.

In other embodiments, vectors useful in the practice of the present invention are microinjected directly into plant cells by use of micropipettes to mechanically transfer the recombinant. In still other embodiments, the vector is transferred into the plant cell by using polyethylene glycol; fusion of protoplasts with other entities, either minicells, cells, lysosomes or other fusible lipid-surfaced bodies; protoplast transformation; direct gene transfer.

In still further embodiments, the vector may also be introduced into the plant cells by electroporation. In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In yet other embodiments, the vector is introduced through ballistic particle acceleration using devices (for example, available from Agracetus, Inc., Madison, Wis. and Dupont, Inc., Wilmington, Del.).

In addition to direct transformation, in some embodiments, the vectors comprising a nucleic acid sequence encoding EfDAcT and/or EkDAcT are transferred using Agrobacterium-mediated transformation. Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Heterologous genetic sequences (for example, nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome. Species that are susceptible infection by Agrobacterium may be transformed in vitro. Alternatively, plants may be transformed in vivo, such as by transformation of a whole plant by Agrobacterium infiltration of adult plants, as in a “floral dip” method.

After selecting for transformed plant material that can express the heterologous gene encoding EfDAcT and/or EkDAcT, whole plants are regenerated. It is known that many plants can be regenerated from cultured cells or tissues, including but not limited to all major species of sugarcane, sugar beet, cotton, fruit and other trees, legumes and vegetables, and monocots (for example, the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos germinate and form mature plants. The culture media will generally contain various amino acids and hormones, such as auxin and cytokinins, Shoots and roots normally develop simultaneously. Efficient regeneration will depend on the medium, on the genotype, and on the history of the culture. The reproducibility of regeneration depends on the control of these variables.

In one embodiment, transgenic plant lines will be established from transgenic plants of the present inventions by tissue culture propagation. In some embodiments, progeny transgenic plants and progeny plant lines will be established using founder transgenic plants in plant breeding programs. In some embodiments, the EfDAcT and/or EkDAcT transgenic plants of the present inventions are heterologous for EfDAcT and/or EkDAcT genes. In other embodiments, the EfDAcT and/or EkDAcT transgenic plants of the present inventions are homozygous for EfDAcT and/or EkDAcT genes. In other embodiments, the presence of nucleic acid sequences encoding a heterologous DAcT of the present invention (including mutants or variants thereof) may be transferred from founder or progeny plants to related plant varieties by traditional plant breeding techniques.

Transgenic plant lines of the present inventions are then utilized for evaluation of oil production and other agronomic traits. In some embodiments, these evaluated plant lines will be used in plant breeding programs for developing commercial varieties and commercial plant lines.

In other embodiments of the present invention, acetyl-TAGS are produced in vitro, from nucleic acid sequences encoding EfDAcT and/or EkDAcT. In other embodiments of the present invention, acetyl-TAGS are produced in vitro, from polypeptides exhibiting EfDAcT and/or EkDAcT-like diacylglycerol acetyltransferase activity.

In some embodiments of the present invention, methods for producing acetyl-TAGS comprise adding an isolated nucleic acid sequence encoding EfDAcT and/or EkDAcT to in vitro expression systems under conditions sufficient to cause production of acetyl-TAGS. The isolated nucleic acid sequence encoding a plant acetyltransferase is any suitable sequence of the invention as described above, and preferably is provided within an expression vector such that addition of the vector to an in vitro transcription and translation system results in expression of the polypeptide. Furthermore, the system contemplated is specific for the translation and function of eukaryotic membrane proteins, that is, it is a microsomal system. The system further comprises the substrates for EfDAcT and/or EkDAcT. Alternatively, the system further comprises the means for generating the substrates for EfDAcT and/or EkDAcT.

In other embodiments of the present invention, the methods for producing large quantities of acetyl-TAGS further comprise collecting the acetyl-TAGS produced. Such methods are known generally in the art. In yet other embodiments of the present invention, the acetyl-TAGS are further purified, as for example by thin layer liquid chromatography, gas-liquid chromatography, high-pressure liquid chromatography, crystallization and/or vacuum distillation.

In some embodiments of the present invention, methods for producing large quantities of acetyl-TAGS comprise incubating EfDAcT and/or EkDAcT under conditions sufficient to result in the synthesis of acetyl-TAGS; generally, such incubation is carried out in a mixture that comprises EfDAcT and/or EkDAcT.

EfDAcT and/or EkDAcT can be obtained by purification of either naturally occurring EfDAcT and/or EkDAcT or recombinant EfDAcT and/or EkDAcT from an organism transformed with heterologous gene encoding EfDAcT and/or EkDAcT. A source of recombinant EfDAcT and/or EkDAcT is either plant, bacterial or other transgenic organisms, transformed with heterologous gene encoding EfDAcT and/or EkDAcT, as described above. The recombinant EfDAcT and/or EkDAcT may include a means for improving purification, as for example a 6×-His tag added to the C-terminus of the protein. Alternatively, EfDAcT and/or EkDAcT is chemically synthesized.

The incubation mixture further comprises substrates for EfDAcT and/or EkDAcT. Alternatively, the inventors contemplate that the mixture further comprises a means for generating substrates for EfDAcT and/or EkDAcT, such as expressing a gene encoding a protein for making more acetyl-TAG substrate available for making more acetyl-TAGS, i.e. increasing the amount of acetyl-TAG substrate for increasing the amount of acetyl-TAGS. Acetyl-TAG substrate is contemplated to be increased by using enzymes, such as using ATP-citrate lyase (EC: 4.1.3.8) to generate acetyl-CoA molecules from a precursor molecule, such as generating acetyl-CoA from citrate. Alternatively, acetyl-CoA synthetase would be used to generate acetyl-CoA from a precursor molecule, such as acetyl-CoA from acetate. As another alternative, phosphatidic acid phosphatase would be used to generate diacylglycerol from phosphatidic acid. As another alternative, phospholipase C would be used to generate diacylglycerol from phospholipids. In other embodiments of the present invention, the methods for producing acetyl-TAGS further comprise collecting the acetyl-TAGS produced.

Further contemplated is that nucleic acids encoding EfDAcT and/or EkDAcT may be utilized to either increase or decrease the level of EfDAcT and/or EkDAcT mRNA and/or protein in transfected cells as compared to the levels in wild-type cells. Such transgenic cells have great utility, including but not limited to further research as to the effects of the overexpression of EfDAcT and/or EkDAcT, and as to the effects as to the underexpression (lower than average of wild-type levels) or a complete lack of EfDAcT and/or EkDAcT.

Accordingly, in some embodiments, expression in plants of nucleic acid sequences encoding a EfDAcT and/or EkDAcT by the methods described above leads to the induced expression and overexpression of EfDAcT and/or EkDAcT in transgenic plants, plant tissues, or plant cells.

Accordingly, in further embodiments, an increase in EfDAcT and/or EkDAcT expression corresponds to increased production of acetyl-TAGS, for example, isolated oils comprise increased amounts of acetyl-TAGS when compared to isolated oils from unmanipulated plants.

In other embodiments of the present invention, nucleic acids encoding lc-TAG synthesizing proteins are utilized to decrease the level of lc-TAG production as compared to wild-type plants, plant tissues, plant cells, or seeds, thus increasing the acetyl-TAG composition of oil. For example, by altering mRNA and/or protein in transgenic plants, plant tissues, plant cells, or seeds lipid synthesizing proteins are altered by increasing or decreasing overall activity. One method of reducing TAG production utilizes expression of antisense transcripts. Antisense RNA has been used to inhibit plant target genes in a tissue-specific manner. Antisense inhibition has been shown using the entire cDNA sequence as well as a partial cDNA sequence. There is also evidence that 3′ non-coding sequence fragment and 5′ coding sequence fragments, containing as few as 41 base-pairs of a 1.87 kb cDNA, can play important roles in antisense inhibition.

Accordingly, in some embodiments, nucleic acid sequences encoding lipid-synthesizing proteins for use in reducing TAG production are oriented in a vector and expressed so as to produce antisense transcripts. To accomplish this, a nucleic acid segment from the desired gene is cloned and operably linked to a promoter such that the antisense strand of RNA will be transcribed. The expression cassette is then transformed into plants and the antisense strand of RNA is produced. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other proteins within a family of genes exhibiting homology or substantial homology to the target gene.

Furthermore, for antisense suppression, the introduced sequence also need not be full length relative to either the primary transcription product or fully processed mRNA.

Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full-length nucleotides should be used, though a sequence of at least about 100 nucleotides is preferred, a sequence of at least about 200 nucleotides is more preferred, and a sequence of at least about 500 nucleotides is especially preferred.

Catalytic RNA molecules or ribozymes can also be used to inhibit expression of the target gene or genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, Solanum nodiflorum mottle virus and subterranean clover mottle virus. Ribozymes targeted to the mRNA of a lipid biosynthetic gene, resulting in a heritable increase of the target enzyme substrate, have also been described.

Another method comprising nucleic acid sequences encoding lipid synthesizing proteins for use in reducing TAG production utilizes the phenomenon of co-suppression or gene silencing. The phenomenon of co-suppression has also been used to inhibit plant target genes in a tissue-specific manner. Co-suppression of an endogenous gene using a full-length cDNA sequence as well as a partial cDNA sequence (730 bp of a 1770 bp cDNA) is known. Accordingly, in some embodiments the nucleic acid sequences encoding a DGAT1, DGAT2, PDAT, etc. polypeptide of the present invention and fragments and variants thereof are expressed in another species of plant to effect co-suppression of a homologous gene.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For co-suppression, the introduced sequence in the expression cassette, needing less than absolute identity, also need not be full-length, relative to either the primary transcription product or fully processed mRNA. This may be preferred to avoid concurrent production in some plants that are overexpressers of the co-suppression cassette. A higher identity in a shorter than full-length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

An effective method to down regulate a gene is by hairpin RNA constructs. Guidance to the design of such constructs for efficient, effective and high throughput gene silencing have been described.

The inventors contemplate expression of a heterologous EfDAcT and/or EkDAcT gene and encoded protein of the present inventions in additional host plants (different plant backgrounds) that would be useful for increasing the proportion of acetyl-TAGS in the seed oil. In one embodiment, a host plant would have low or reduced lc-TAG production. Multiple methods are available for reducing lc-TAG production in plants for use as host plants for inserting EfDAcT and/or EkDAcT genes of the present inventions for producing oil with a high acetyl-TAG content. In some embodiments, cultivars of oil crop plants are identified with naturally low production of lc-TAGs in comparison to wild-type plants for use as a host plant. In other embodiments, plants are mutagenized for reducing lc-TAG production for use as a host plant. In yet further embodiments, plants are engineered for reducing lc-TAG production, for example, using RNAi for inhibiting gene expression of proteins providing substrates or inhibiting gene expression of proteins with direct lc-TAG production for use as a host plant. Indeed, it is contemplated that multiple methods of reducing 1 lc-TAG production may be employed together to produce the host plant for EfDAcT and/or EkDAcT expression. Thus, the inventors contemplate using certain plants with alterations in their genetic capability to synthesize lc-TAGs as hosts for heterologous EfDAcT and/or EkDAcT expression. In other embodiments, homologous EfDAcT and/or EkDAcT expression is contemplated. In yet further embodiments, inducible EfDAcT and/or EkDAcT expression is contemplated.

In some embodiments, alterations in lc-TAG production may be found naturally in plants, for example, a natural variation in lc-Tag production found within and between variants, cultivars and populations of plants (i.e. species and varieties or variations found within species and varieties), such as Arabidopsis plants, Camelina plants, soybean plants, Brassica species, including B. napsus plants. In other embodiments, alterations in seed oil fatty acids were or are induced by mutation. In yet further embodiments, alteration in lc-TAG production may be induced by genetic engineering. In additional embodiments, alterations in lc-TAG production are contemplated to result from a combination of alterations, for example, in one embodiment, identifying a naturally low lc-TAG producing plant for use in mutational and/or genetic engineering for producing oil with a high acetyl-TAG content.

The strategy for genetic engineering contemplated by the present invention for any particular plant is on a species by species basis, i.e. some plant species will require different compositions and/or method for increasing acetyl-TAG production for use in the present invention, such different compositions and methods being described herein. For example, a plant of the Brassicea family, where in general Brassicea plants depend on DGAT1 for lc-TAG synthesis, would require genetic manipulation associated with DGAT1 activity. In contrast, in an oilseed plant that is not a member of the Brassicaceae family it may be more important to silence activity associated with the DGAT2 gene. For example, in castor (Ricinus communis L.) the most strongly expressed TAG-synthesis gene in seeds is the DGAT2 gene. DGAT2 expression was induced 18-fold during seed maturation, whereas DGAT1 was barely induced.

One specific example of such a host plant contemplated for use in the present inventions, as a plant affected in its ability to synthesize endogenous lc-TAGs is an Arabidopsis plant line having a mutation in a gene associated with lowering lc-TAG production. Such a mutation is contemplated in any one or more of a DGAT1 gene, a PDAT gene, and combinations of mutations in more than one gene within the same plant. Examples of combination mutations in plants that may find use in the present inventions are host plants with double mutations comprising a mutation in at least one mutation in its DGAT1 gene in combination with a mutation in its PDAT gene. In some embodiments, mutations include deletion mutants. In yet other embodiments, other genetic combinations in plants include complete null plants, such that plants have low or undetectable DGAT1 activity and low or undetectable PDAT expression.

In another embodiment, a plant with low lc-TAG production will be engineered or bred to increase the level of substrates for acetyl-TAG production. For example, embodiments are contemplated to comprise altered expression, i.e. increased or decreased expression of a gene for the goal of increasing substrate levels for further increasing acetyl-TAG production.

An exemplary method for one embodiment of increasing (i.e. enhancing) levels of acetyl-TAGS in transgenic Arabidopsis plants by expression of EfDAcT and/or EkDAcT in mutant plants (plants with a mutant, nonwild-type background) comprises using host plants expressing mutant genes which reduce levels of lc-TAGs in seeds.

In one contemplated embodiment, host plants comprising lowered FAE1 expression, naturally or induced, would have a higher percentage and/or amount of acetyl-TAG containing oil in their seeds. The comparison would be to oil isolated from the same amount (by weight) of seeds from wild-type plants or from other plants with wild-type backgrounds consisting of expression of EfDAcT and/or EkDAcT on a background of wild-type expression of FAE1.

In one contemplated embodiment, a host plant comprising lowered VLCFAs are Canola producing rapeseed plants are contemplated for use in combination with expression of an EfDAcT and/or EkDAcT gene of the present inventions for producing oil with high amounts of acetyl-TAGS. As one example, fae1 gene mutations or molecular genetic strategies that eliminate the elongase function of this gene result in greatly reduced long-chain (C20, C22) fatty acid in seed oils. One example of the use of seed oils from plants bred for reduced long-chain fatty acids was the development of Canola oil (low erucic acid rape seed, LEAR) producing varieties of rapeseed plants. Edible oil extracted from LEAR plants is Canola oil essentially devoid of VLCFAs. LEAR containing Canola oil is viewed as the preferred edible seed oil over wild type rapeseed (HEAR) oil with high amounts of VLCFAs. Rapeseed plants with LEAR were analyzed and found to comprise deletion mutant fae1 genes associated with low VLCFAs. Thus in one embodiment, a host plant is a Canola oil producing plant. In another embodiment, the hose cell is a cell obtained from a Canola oil producing plant.

EfDAcT and/or EkDAcT genes and encoded polypeptides find use in the present inventions for producing oils comprising acetyl-TAGS contemplated for use as a biofuel. For example, current and future cultivation of oil-seed crops producing acetyl-TAGS by compositions and methods of the present inventions (either transgenic introduction or through plant breeding) are contemplated to provide a new biofuel with improved properties and production compared with existing biodiesel products and oils used in engines (such as jet engines, etc.). Primary advantages of using genes and polypeptides of the present inventions for providing novel oils include increasing economic viability for using natural oils by eliminating (or reducing) processing (transesterification) time and costs. In other words, reducing or eliminating alcohol modification of plant oils prior to use. In some embodiments, oils comprising acetyl-TAGS of the present inventions are contemplated for use combined with conventional fuel or in combination with other types of biofuels.

Thus the inventors contemplated that acetyl-TAGS produced by EfDAcT and/or EkDAcT genes and encoded polypeptides expressed in plants and cells other than Euonymus fortunei or Euonymus kauschovicus would also be useful for producing novel oils. These novel oils comprising acetyl-TAGS would contain acetyl-TAGS in amounts higher than found in plants and cells not expressing Euonymus genes and encoded polypeptides. Thus, genes and polypeptides for producing acetyl-TAGS are contemplated to provide oils for commercial use in bioenergy, machine oil, oleochemical, and nutritional fields. Acetyl-TAGS produced by Euonymus genes and encoded polypeptides in novel oils, are contemplated to have utility for use, either as components of isolated oils or as isolated lipids, as biofuels and biolubricants and for use as oleochemicals and in food products.

In some embodiments, oils produced by cells and whole organisms (such as transgenic cells and organisms) expressing heterologous acetyl-TAGS genes and encoded polypeptides of the present inventions are contemplated for use as additives in diesel and gasoline fuels in automotive or airplane industries. Fuel additives are usually used in automotive fuels, such as gasoline and diesel, to help meet the fuel specifications and improve fuel and engine performance. In some embodiments, oils produced by cells and whole organisms (such as transgenic cells and organisms) expressing heterologous acetyl-TAGS genes and encoded polypeptides of the present inventions are contemplated for use as diesel additives, for example, as cetane improvers, lubricity improvers, wax modifiers, and the like. In some embodiments, oils produced by cells and whole organisms (such as transgenic cells and organisms) expressing heterologous acetyl-TAGS genes and encoded polypeptides of the present inventions are contemplated as gasoline additives, for example as deposit control additives, anticorrosion additives, antioxidant additives, and the like.

Oils comprising acetyl-TAGS produced by genes and polypeptides (amino acid sequences) of the present inventions are contemplated for use as polymer feedstock. For example, oils comprising acetyl-TAGS are contemplated to provide novel feedstock for polymers to replace conventional. TAGs. Further, acetyl-TAG comprising oils of the present inventions are contemplated to provide new polymers with new properties.

It is contemplated that the acetyl-TAG oils would be replacements oils used for fuel in machinery that uses heavy-duty diesel engines, such as in shipping, railroad locomotive and heavy earth-moving machinery, which can more readily tolerate higher viscosity fuels. Thus, isolated oils comprising acetyl-TAGS produced by heterologous EfDAcT and/or EkDAcT genes and proteins, including homologous of genes encoding proteins at least 95% identical to SEQ ID NO:1 and/or SEQ ID NO:2, of the present inventions are contemplated for use directly in diesel engines.

Vegetable oils, comprising primarily long chain acyl groups provide excellent lubricity and were used as base fluids for a variety of lubricant applications. In these applications the vegetable oil was formulated with an additive package to bring its performance up to the specification required for a particular application. Additive packages include dispersants, detergents, antiwear and anticorrosion inhibitors, friction modifiers, antioxidants, viscosity enhancers, antifoaming agents and pour point depressants.

The inventors contemplated that oils comprising primarily acetyl-TAGS should provide a lower viscosity base fluid to blend into or completely replace current vegetable oil formulations based on lc-TAG. Thus it is anticipated that oils comprising primarily acetyl-TAGS, will show enhanced lubricity when compared to a medium chain TAG-based vegetable oil such as coconut or palm kernel oil, which contains predominantly medium-chain saturated fatty acids. Thus in one embodiment, the inventors contemplate vegetable oils produced by host cells and plants of the present inventions having performance measurements closer than wild-type vegetable oils to values required for use as lubricants.

One advantage of using TAGs in general as a lubricant feedstock or as a base stock for mixing with other types of oils is their complete biodegradability. Thus in another embodiment, the acetyl-TAGS of the present inventions are contemplated for use as base fluids in lubricants, and in particular a lubricant for use on or in engines. In one embodiment, where lubricants are immediately lost to the environment (for example, chainsaw engines, marine engines, and the like) the inventors contemplate the use of a lubricant comprising an acetyl-TAG, oil, etc., of the present inventions that would undergo rapid biodegradation without residual toxic products. In another embodiment, the inventors contemplate the use of an acetyl-TAG, oil, etc., where the oxidative load during use is relatively mild (for examples, hydraulic fluids, textile or food processing machinery).

Further, an acetyl-TAG oil of the present invention is contemplated to contain monounsaturated fatty acids at the sn-1 and sn-2 position, and thus the acetyl-TAG base fluid will have a much better lower temperature performance than a base fluid based on currently available medium-chain TAGs, and at least equivalent to existing unsaturated lc-TAG vegetable oils. And finally, because current unsaturated lc-TAG vegetable oils contain a large fraction of molecules with three unsaturated fatty acids, whereas acetyl-TAG contains only molecules with two unsaturated fatty acids, the thickening and formation of residues from the base fluid by thermal and oxidative polymerization processes is likely to be significantly reduced.

TAGs are polymerized for use in a variety of industries. For example, triolein or trilinolein form cross-linked thermosetting polymers via metathesis. In contrast, acetyl-TAGS lacking a third long acyl chain are contemplated to form linear thermoplastic polymers. Likewise, consider oils rich in hydroxy fatty acids, such as castor oil, which can be used for the synthesis of polyurethanes. Castor oil is rich in triricinolein (Propane-1,2,3-triyl tris(12-hydroxyoctadec-9-enoate), stereoisomer; CAS #2540-54-7). When triricinolein was reacted with a diisocyanate it produced a cross-linked polyurethane of a certain level of thermoplastic properties. However, when a polyurethane with additional thermoplastic properties was desired, then an acyldiricinolein feedstock was necessary for the reaction. It is extremely unlikely that a vegetable oil enriched in triricinolein could be engineered to produce just diricinoleoyl TAG species, because even if the balance of hydroxylation to oleic fatty acid production could be controlled to give a 2:3 molar ratio, the oleic acid moiety being the precursor to ricinoleic acid, the product TAGs would almost certainly be a mix of mono-, di- and tri-ricinoleoyl species. However, using EfDAcT and/or EkDAcT, a seed producing predominantly triricinolein could be converted to produce acetyldiricinolein with a simple gene engineering strategy of knocking out the endogenous TAG synthesizing genes and replacing them with EfDAcT and/or EkDAcT induced lipids. Thus, another contemplated use for oils comprising acetyl-TAGS produced by heterologous EfDAcT and/or EkDAcT genes and proteins, including homologous of genes encoding proteins at least 95% identical to SEQ ID NO.:1 and/or SEQ ID NO:2, of the present inventions are as oleochemical feed stocks for the modulation of polymer properties in the production of such polymers.

Modified triacylglycerols were developed commercially and used as reduced calorie oils. For example, SALATRIM consists of saturated fatty acids and short-chain fatty acids esterified to glycerol whereas ECONA/ENOVA is a mixture of acylglycerols dominated by 1,3 diacylglycerols. These reduced calorie oils are currently synthesized using chemical and enzyme catalysts. With a similar chemical structure, from the viewpoint that acetyl-TAGS contain at least one short-chain fatty acid in place of a medium or long chain, acetyl-TAGS alone or in mixtures with longer chain fatty acids, are contemplated to represent an alternative form of these existing reduced calorie oils. However, in contrast to currently used oils, oils comprising acetyl-TAGS are contemplated to have numerous advantages over known reduced calorie oil substitutes. Thus in one embodiment, plant oils produced by heterologous EfDAcT and/or EkDAcT genes and proteins in plants, including homologous of genes encoding EfDAcT and/or EkDAcT and EfDAcT and/or EkDAcT-like proteins at least 95% identical to SEQ ID NO:1 and/or SEQ ID NO:2, of the present inventions are contemplated for use as edible oils. In another embodiment, plant oils of the present inventions are directly extracted from seed oil crops capable of producing these molecules. Isolation methods include but are not limited to cold pressing, by hand or machine, and the like. Thus isolation of oils of the present inventions is contemplated to be more economical to produce than currently produced commercial oils. In yet a further embodiment, direct isolation of oils of the present inventions are contemplated to reduce or eliminate the cost of processing. In an additional embodiment, oils of the present inventions are potentially more attractive from a consumer standpoint, i.e. fewer unpleasant or unwanted health side effects induced by current reduced calorie oils.

Oils comprising acetyl-TAGS produced by genes and polypeptides (amino acid sequences) of the present inventions are further contemplated for use as a food ingredient. For example, oils comprising acetyl-TAGS are contemplated to provide lower calorie content compared to conventional TAG oils in addition to a niche use in producing reduced calorie foods or as novel cooking oil. In some embodiments, the oils of the present inventions are contemplated for use in food processing applications such as baking, sprays, and food machinery lubricants. Another advantage of using acetyl-TAG to reduce calorie intake from fat arises from the reduced viscosity of acetyl-TAGS, allowing fat to more effectively drain from deep-fried foods after the frying step.

In another embodiment, the inventors contemplate a new low calorie food ingredient with lower cost. In particular, the lower calorie content of acetyl-TAGS when compared to equivalent conventional TAGs (due to one less long chain fatty acid) provides an opportunity to produce natural plant oils for use in the food industry. In a preferred embodiment, foods comprising acetyl-TAGS produced by compositions and methods of the present inventions would have lower calorie content without the need for chemical modification. In one embodiment, an EfDAcT and/or EkDAcT gene and polypeptide of the present inventions is contemplated for expression in a transgenic oil-seed crop plant, for example, in a food-approved species such as soybeans, canola etc., to provide a commercial source of oil from which acetyl-TAGS would be extracted (isolated).

In another embodiment, oils for use in food preparation and as a part of a food product are contemplated for use after isolation from plant parts expressing heterologous EfDAcT and/or EkDAcT proteins. Thus, the inventors contemplated that expression of a heterologous EfDAcT and/or EkDAcT gene and encoded protein of the present inventions would be useful for producing novel oils in plant parts such as leaves. Because acetyl-TAGS are not usually produced in Arabidopsis plants, Arabidopsis leaves as models for Brassica plants will be chosen for testing ectopic transfection and expression of a heterologous EfDAcT and/or EkDAcT gene and encoded protein for producing acetyl-TAGS in leaves of plants.

GATEWAY technology or GoldenBraid technology is contemplated to be used for transferring a EfDAcT and/or EkDAcT gene from an entry vector to a plant binary vector where the gene will be expressed under the control of a constitutive promoter, such as a CMV 355 promoter. This construct will be transformed into Arabidopsis leaves using Agrobacterium mediated transformation.

In other embodiments, expression of EfDAcT and/or EkDAcT will be used for making transgenic plants of the present inventions where the DAcT gene is under control of plant part specific promoter, such as a leaf promoter.

Lipids will be extracted from the leaves of transgenic plants from either ectopic transfection or from leaves harvested from whole transgenic plants. TAG content will be determined using ESI-MS as described herein.

In a preferred embodiment, a plant for use in making an acetyl-TAG oil for human consumption is a plant currently used for providing edible oils from seeds and plant parts, include but are not limited to a Brassica napus plant (providing Canola oil), a soybean plant (providing soybeans and soybean oil), and the like.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth experiments characterizing the enzymes and their uses in accordance with embodiments of the present invention. It is to be understood, however, that these examples are provided by way of illustration and nothing therein should be taken as a limitation upon the overall scope of the invention.

### Example I

Acetyltransferases from various plant species were isolated that synthesize acetyl-triacylglycerols, allowing identification of conserved residues important for acetyltransferase activity.

### Introduction

The membrane bound O-acyltransferase (MBOAT) family comprises integral membrane enzymes that catalyze the transfer of the fatty acid moiety from an acyl-CoA to a variety of lipid and protein substrates. Most characterized MBOATs acylate lipids. For example, acyl-CoA: cholesterol acyltransferases (ACAT) attach fatty acids to cholesterol and type 1 diacylglycerol acyltransferases (DGAT1) acylate the sn-3 position of diacylglycerol (DAG) to form triacylglycerols (TAG). Other enzymes are involved in the remodeling of the fatty acid composition of membranes through their activity towards lysophospholipids. Additionally, a few MBOATs transfer fatty acids to protein and peptide substrates, typically to activate biological function. This group includes the mouse enzyme Porcupine that adds the monounsaturated fatty acid palmitoleate to a key serine of Wnt-3a in order for this signaling protein to be secreted. Similarly, Hedgehog acyltransferase (Hhat) palmitoylates Sonic hedgehog as part of the signaling protein's post-translational processing. Ghrelin O-acyltransferase (GOAT) adds octanoic acid to the hormone peptide ghrelin, thus allowing it to stimulate weight gain and modulate glucose metabolism.

The Euonymus alatus diacylglycerol acetyltransferase (EaDAcT) catalyzes the acetylation of the sn-3 hydroxyl group of DAG to form 3-acetyl-1,2-diacyl-sn-glycerol (acetyl-TAG). The presence of an acetyl group instead of a longer fatty acyl group gives acetyl-TAG altered physical properties compared to regular triacylglycerols (here referred to as 1cTAG), including reduced viscosity and lower melting temperatures. EaDAcT's preferred substrate is DAG, but it is also capable of acetylating fatty alcohols to form alkyl acetates, albeit with reduced efficiency. This weak wax synthase activity is consistent with the enzyme's sequence similarity to the jojoba wax synthase and distances EaDAcT from the DGAT1 enzymes with which it shares a common DAG substrate.

In this study, we isolated additional acetyltransferases from other acetyl-TAG producing species which together possess a range of enzyme activity levels, some of which were unexpectedly higher than that of EaDAcT.

### Experimental Procedures

### Seed Collection and RNA Isolation

Seeds were collected from local plants or purchased from commercial seed suppliers. Total RNA was isolated either from endosperm tissue (Celastraceae species) or from whole seeds (Adonis aestivalis) using an established procedure with the following modifications. Ground tissue was incubated in extraction buffer (2% hexadecyltrimethylammonium bromide, 2% polyvinylpryrrolidone K 30, 100 mM Tris-HCl pH 8.0, 25 mM EDTA, 2M NaCl, 0.5 g/L spermidine, 2% B-mercaptoethanol) at 65° C. for 5 min. Protein and DNA were removed by extracting with 1:1 phenol:chloroform (v/v) twice. The final RNA-containing aqueous phase was mixed with LiCl to a final concentration of 3M and incubated at −20° C. overnight. RNA was precipitated by centrifuging at 11,000 g for 2 h and then washed with 70% ethanol.

### Illumina Sequencing and Computational Analysis

Total RNA was prepared for sequencing using Illumina TruSeq RNA kits and then sequenced on an Illumina HiSeq 2000 system to obtain 100 bp paired-end reads. Reads were stringently pre-cleaned with PRINSEQ version 0.20.3. Single k-mer assemblies were performed for k=27 up to k=57 with a step size 10 with Velvet version 1.2.08 and Oases version 0.2.08. Single k-mer assemblies were merged Oases using k=27. Merged assembly transcripts were clustered with MIRA version 3.4 and CDHit version 4.6.1. BLASTX version 2.2.26 was run to search assembled contigs for putative homologs to the Arabidopsis genome. The script OrthologHitRatio.pl was used to estimate percent of the full protein coding sequence represented in the contig, or the ortholog hit ratio (OHR). Assemblies were filtered for contigs >400 bp and aligned used Bowtie2 version 2.1.0. Alignment counts were summarized using Count_reads_denovo.pl version 1.0. BLASTP was used to search the assembled contigs for sequences with high similarity to EaDAcT, which were selected for cloning. Protein sequences were aligned using Multiple Sequence Comparison by Log-Expectation (MUSCLE). Phylogenetic analysis was performed with MEGA6 using the neighbor-joining method with the Poisson model. Divergence times for all branching points in the topology were calculated with RelTime using the branch lengths contained in the inferred tree. The topological organization of EaDAcT was predicted using the following algorithms: TOPPRED 2, TOPCONS, OCTOPUS, SCAMPI, TMfinder, MEMSAT-SWM, MEMSAT-3.

### Cloning, Expression and Mutagenesis of Acyltransferase Genes

Primers (GGAAGAAGCCGGTGATTGATGAAAG (SEQ ID NO: 20) and TTGGAGGTGGAGATGAAGTGTAAG (SEQ ID NO: 21)) were used to amplify the coding regions of DAcT genes from cDNA isolated from different Celastraceae species. The 5′ and 3′ regions of these genes were then cloned with Rapid Amplification of cDNA Ends (RACE) kit. Transcription initiation sites were predicted using the NetStart 1.0 Prediction server. First strand cDNAs were synthesized using oligo dT primer and reverse transcriptase (SuperScript III, Invitrogen). Gene sequences containing full coding regions of DAcT orthologs were amplified from cDNA using Phusion polymerase (Thermo Fisher Scientific, MA) and primers corresponding to the 5′ and 3′ end regions, and were cloned into the yeast expression vector pYES2/CT (Invitrogen), with the hemagglutinin epitope coding sequence inserted to the 3′ end of the gene sequences prior to the stop codon. Site directed mutations were introduced using the Phusion site-directed mutagenesis kit (New England Biolabs, MA). Primers used for cloning and mutagenesis are listed in Table 1. All acetyltransferase genes were expressed in the TAG-deficient, quadruple knockout S. cerevisiae strain H1246.

TABLE 1

| Name | | Sequence |
|---|---|---|
| (SEQ ID NO:) | Purpose | (5′ to 3′) |
| 5′-EtDacT | Cloning EtDAcT to | ttggagctcATGA |
| (SEQ ID NO: 22) | pYES2/CT vector at | TGGATGCTCATCG |
| | SacI and XhoI site | AGAG |
| 3′-EtDAcT | | tagactcgagTCA |
| (SEQ ID NO: 23) | | ATTTCCACACATA |
| | | AAACTTG |
| 5′-EbDacT | Cloning EbDAcT to | ttggagctcATGA |
| (SEQ ID NO: 22) | pYES2/CT vector at | TGGATGCTCATCG |
| | SacI and XhoI site | AGAG |
| 3′-EbDAcT | | tagactcgagTCA |
| (SEQ ID NO: 24) | | ATTTCCACACATA |
| | | AACCTTG |
| 5′-EkDAcT | Cloning EkDAcT to | ttggagctcATGA |
| (SEQ ID NO: 25) | pYES2/CT vector at | TGGATGTTCATCA |
| | SacI and XhoI site | AGAG |
| 3′-EkDAcT | | tagactcgagTCA |
| (SEQ ID NO: 26) | | ATTTCCACAGATA |
| | | AACCTTG |
| 5′-EfDAcT | Cloning EfDAcT to | ttggagctcATGA |
| (SEQ ID NO: 25) | pYES2/CT vector at | TGGATGTTCATCA |
| | SacI and XhoI site | AGAG |
| 3′-EfDAcT | | tagactcgagTCA |
| (SEQ ID NO: 26) | | ATTTCCACAGATA |
| | | AACCTTG |
| 5′-CsDAcT | Cloning CsDAcT to | ttggagctcATGA |
| (SEQ ID NO: 27) | pYES2/CT vector at | TGGATTTCAATCA |
| | SacI and BstI site | AG |
| 3′-CsDAcT | | gaaccactgtgct |
| (SEQ ID NO: 28) | | ggCGTCAATCCTG |
| | | TTCCAA |
| 5′-AaDAcT | Cloning AaDAcT to | caggtaccATGGG |
| (SEQ ID NO: 29) | pYES2/CT vector at | AGGTGAACTGAGG |
| 3′-AaDAcT | KpnI and XmaI site | aattcccgggGCT |
| (SEQ ID NO: 30) | | TGTGGCAAAGATT |
| | | G |
| 5′-EaDAcT-S253A | Generating EaDAcT | ACATTTGTCGTAg |
| (SEQ ID NO: 31) | that has serine at | ctGGAGTTATGCA |
| | position 253 | T |
| 3′-EaDAcT-S253A | replaced by | TGCAATTATAGCT |
| (SEQ ID NO: 32) | alanine | GGAAAATAAGCCC |
| | | A |
| 5′-EaDAcT-H257A | Generating EaDAcT | TCAGGAGTTATGg |
| (SEQ ID NO: 33) | that has histidine | ctGATGTAGTGTA |
| | at position 257 | CT |
| 3′-EaDAcT-H257A | replaced by | TACGACAAATGTT |
| (SEQ ID NO: 34) | alanine | GCAATTATAGCTG |
| | | G |
| 5′-EaDAcT-D258A | Generating EaDAcT | TCAGGAGTTATGC |
| (SEQ ID NO: 35) | that has aspartate | ATgctGTAGTGTA |
| | at position 258 | CTAT |
| 3′-EaDAcT-D258A | replaced by | TACGACAAATGTT |
| (SEQ ID NO: 34) | alanine | GCAATTATAGCTG |
| | | G |
| 5′-EaDAcT-ΔV263 | Generating EaDAcT | TACATGATGCATA |
| (SEQ ID NO: 36) | that has valine at | TGTATCCCAAGTG |
| | position 263 | G |
| 3′-EaDAcT-ΔV263 | deleted | ATAGTACACTACA |
| (SEQ ID NO: 37) | | TCATGCATAACTC |
| | | CT |
| 3′-EaDAcT-D258N | Reverse primer to | TCAGGAGTTATGC |
| (SEQ ID NO: 38) | generate EaDAcT | ATaatGTAGTGTA |
| | that has aspartate | CTAT |
| | at position 258 | |
| | replaced by | |
| | asparagine | |
| 3′-EaDAcT-D258E | Reverse primer to | TCAGGAGTTATGC |
| (SEQ ID NO: 39) | generate EaDAcT | ATgaaGTAGTGTA |
| | that has aspartate | CTAT |
| | at position 258 | |
| | replaced by | |
| | glutamate | |

### Microsome DGAT Assay and Lipid Analysis

Microsomal protein isolation and in vitro DGAT assays were performed, except that galactose-mediated induction of protein expression occurred for 24 h, and the DGAT assay reactions contained 15 μg of microsomal protein and were incubated at 30° C. for 15 min. Neutral lipids were extracted from plant tissues and dried yeast cells, except that yeast cells were collected after 24 h of induction. TAGs were quantified using ESI-MS.

### Immunoblotting and Protein Quantification

Resolved proteins from SDS-PAGE were transferred to nitrocellulose membrane and incubated with the appropriate primary antibodies at the following dilutions: anti-HA antibody (1:10,000; clone 2-2.2.2.14, Thermo Scientific), anti-Kar2 (1:1000; clone sc-33630, Santa Cruz Biotechnology), anti Pma1 antibody (1:500), anti-Sec61 antibody (1:1,000), and anti c-Myc antibody (1:3000; Santa Cruz Biotechnology) followed by incubation with secondary antibodies. For protease protection assays, membranes were incubated with goat anti-rabbit IgG-HRP and rabbit anti-mouse IgG horse radish peroxidase conjugated secondary antibodies and the antibodies were detected using SuperSignal Wes Pico Chemiluminescent substrate (Thermo Scientific). For other experiments involving protein quantification, membranes were probed with goat anti-rabbit IgG Dylight™488 and goat anti-mouse IgG Dylight™633 antibodies (Thermo Scientific). The blots were scanned on a Typhoon scanner (model 9410; Amersham), with the signal for EaDAcT-HA obtained using the 633 nm laser and signals for Sec61 and Pma1 obtained using the 488 nm laser. Protein abundance was quantified using densitometry analysis with ImageQuantTL software (GE Healthcare Life Science).

### Results

### Seeds from Divergent Plant Species Produce Acetyl-TAG

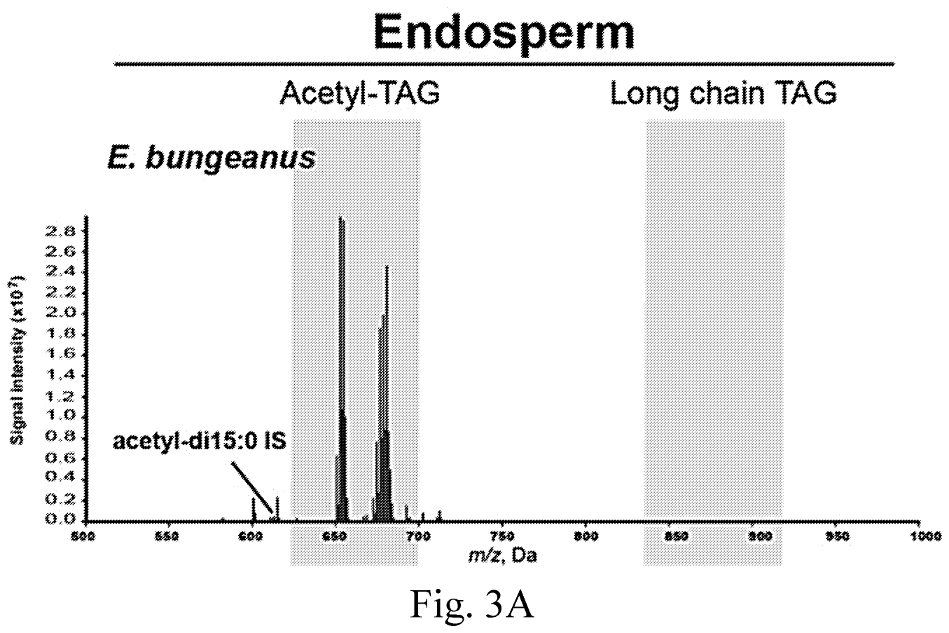

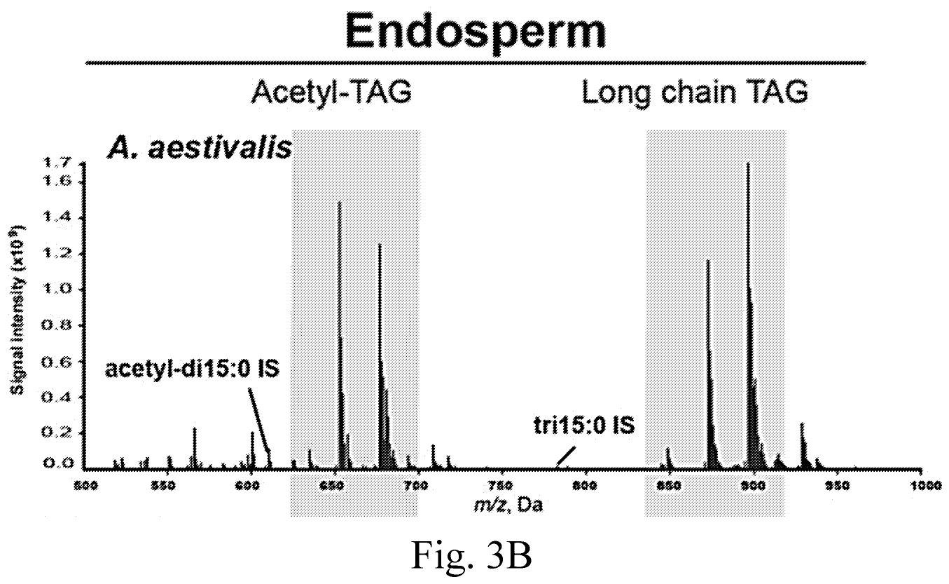

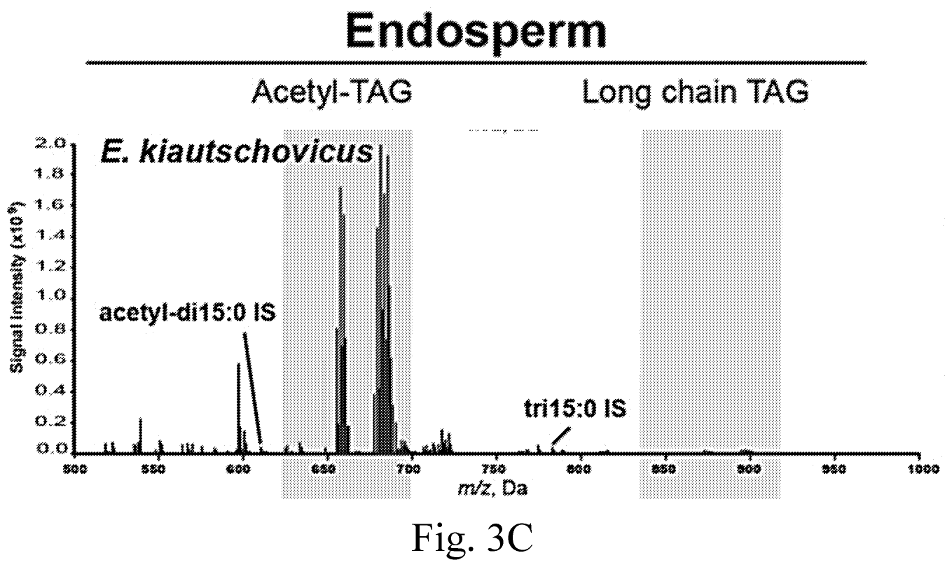

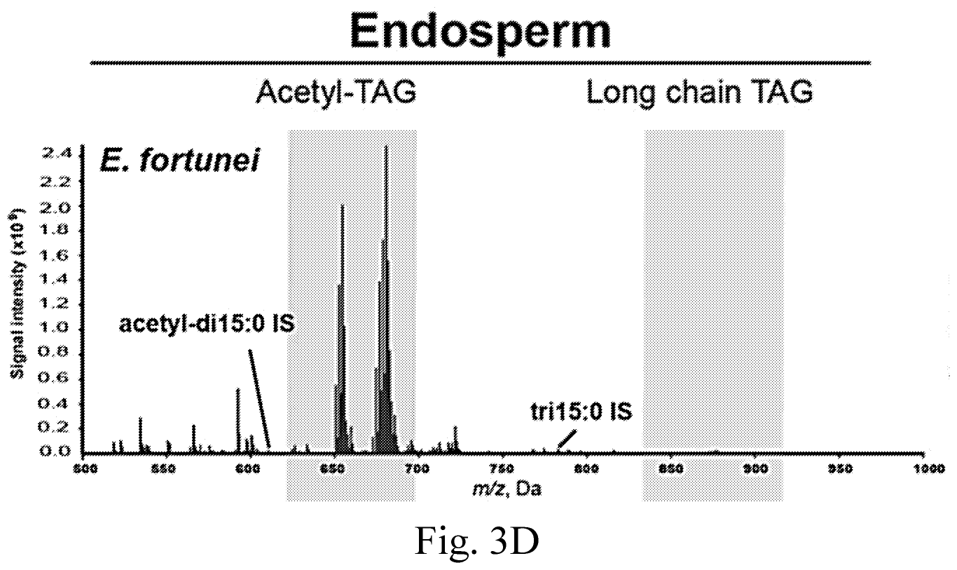

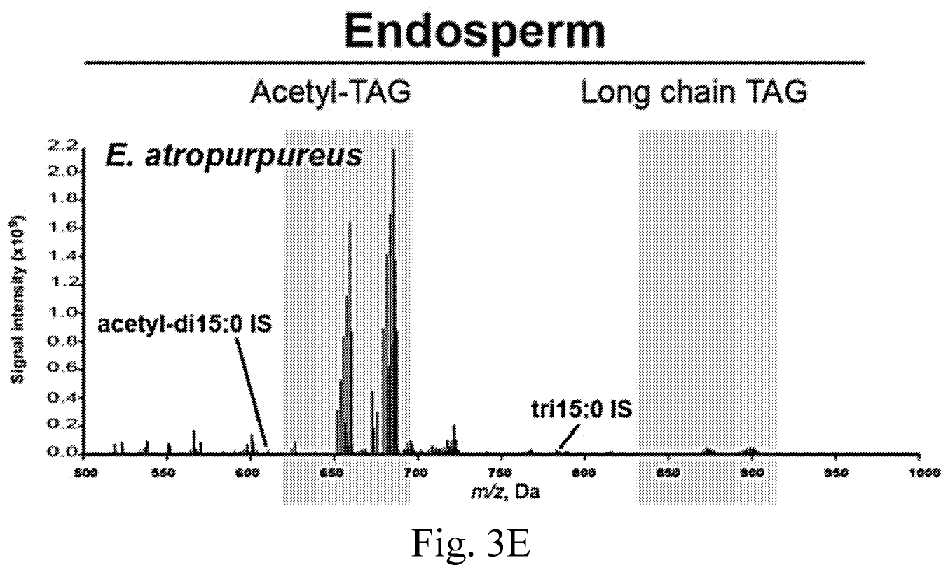

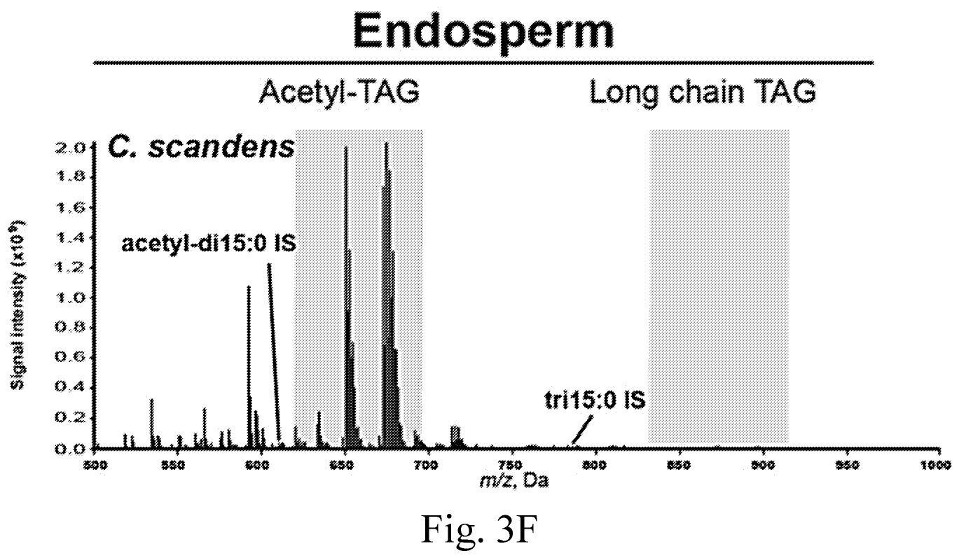

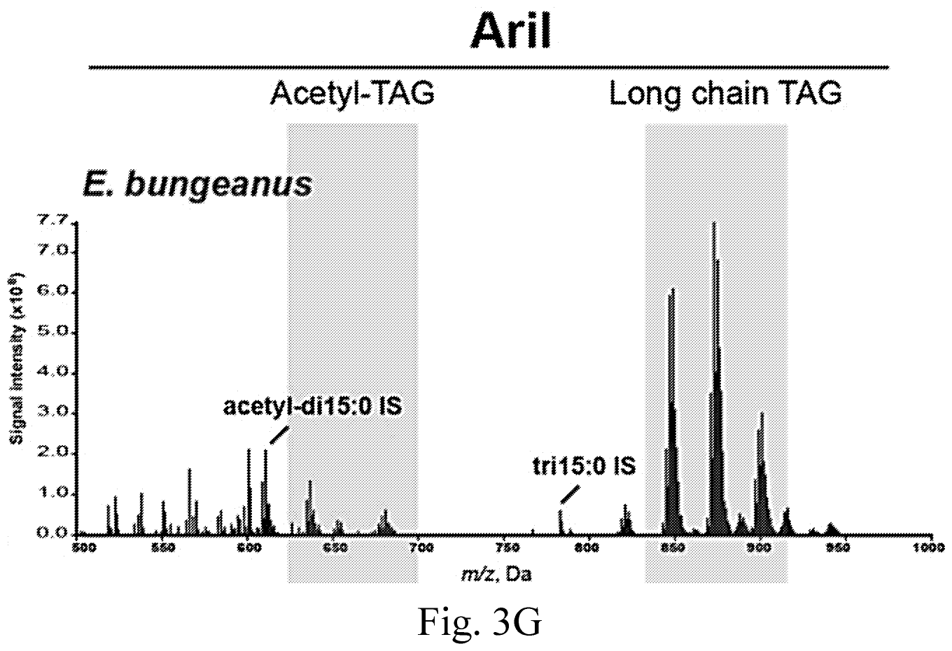

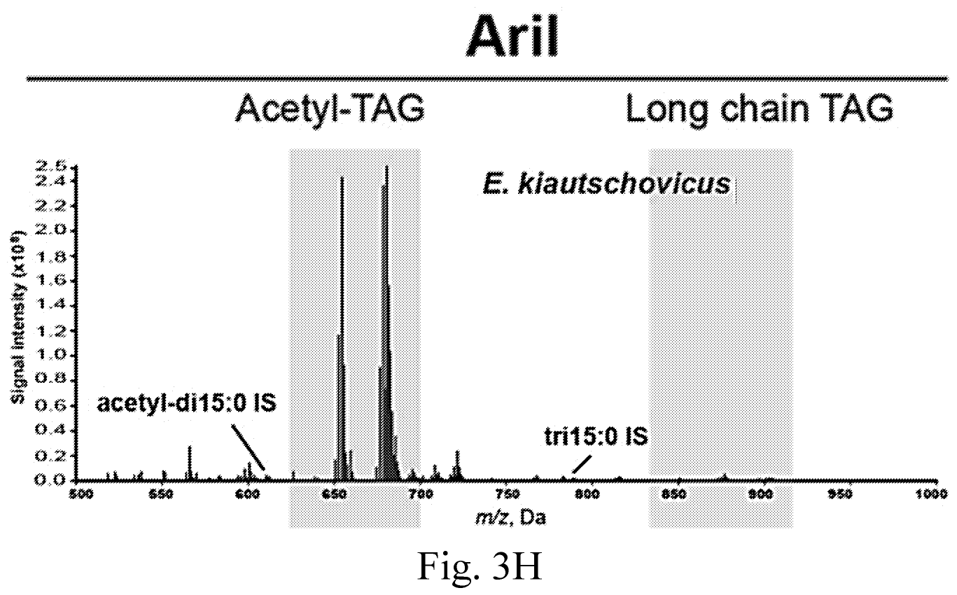

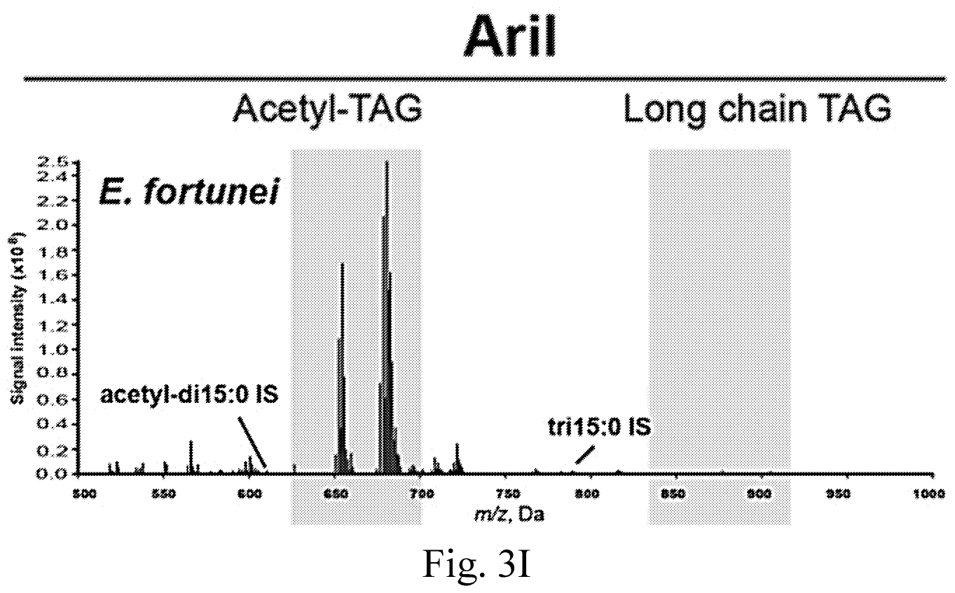

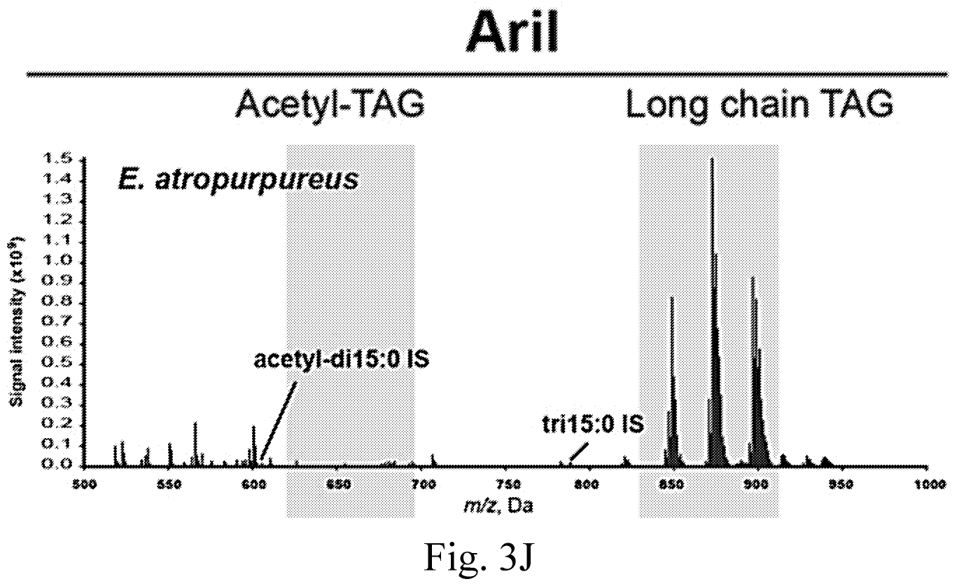

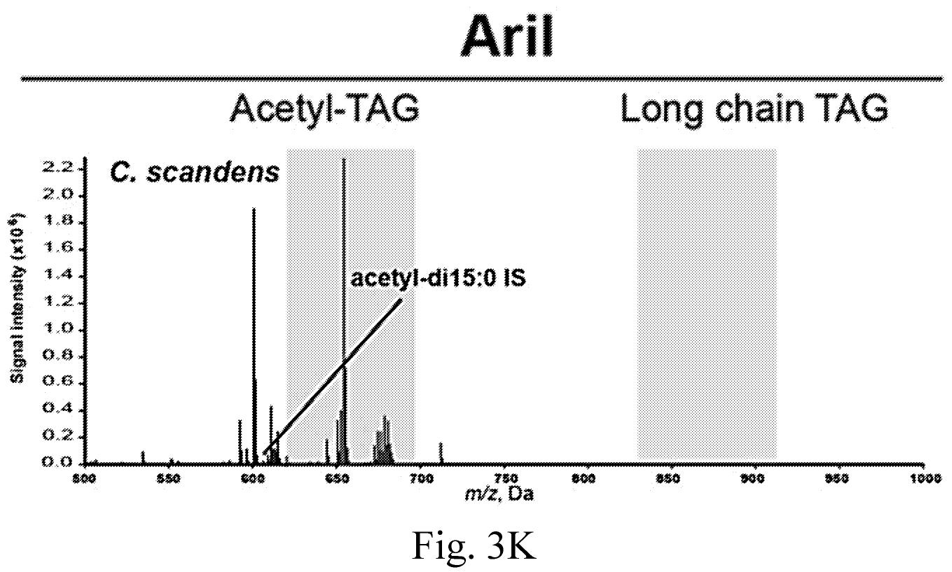

We were interested in identifying residues important for the extreme substrate specificity of EaDAcT, which preferentially uses the shortest acyl-CoA, acetyl-CoA. We hypothesized that residues important for acetyltransferase activity are conserved among DAG acetyltransferase enzymes present in the phylogenetically diverse species that produce acetyl-TAG. Most of these species belong to the Celestraceae plant family (Euonymus and Celastrus species); others are members of the Ranunculales and Ericales orders and therefore are distantly related to the Celestraceae family. The presence of acetyl-TAG in seeds of Celastrus scandens, E. bungeanus, E. atropurpureus, E. fortunei, E. kiautschovicus, and Adonis aestivalis was confirmed by electrospray ionization mass spectrometry (ESI-MS). FIGS. 3A-3K show positive-ion electrospray ionization mass spectra of neutral lipid extracts from aril and seed tissue of different species indicating the presence of acetyl-TAG (left) and long chain TAG (right), wherein tripentadecanoin (tri15:0) was added during lipid extraction and 3-acetyl-1,2-dipentadecanoyl-sn-glycerol (acetyl-di15:0) was added prior to ESI-MS. In all species except for A. aestivalis, acetyl-TAG are the major storage lipids present while regular TAG are present at relatively low levels in endosperm tissues. Interestingly, while regular TAG predominate in the aril layer of E. bungeanus and E. atropurpureus, the aril layer of E. fortunei, E. kiautschovicus, and C. scandens contained mostly acetyl-TAG.

### Novel Acyltransferases Possess Acetyltransferase Activity

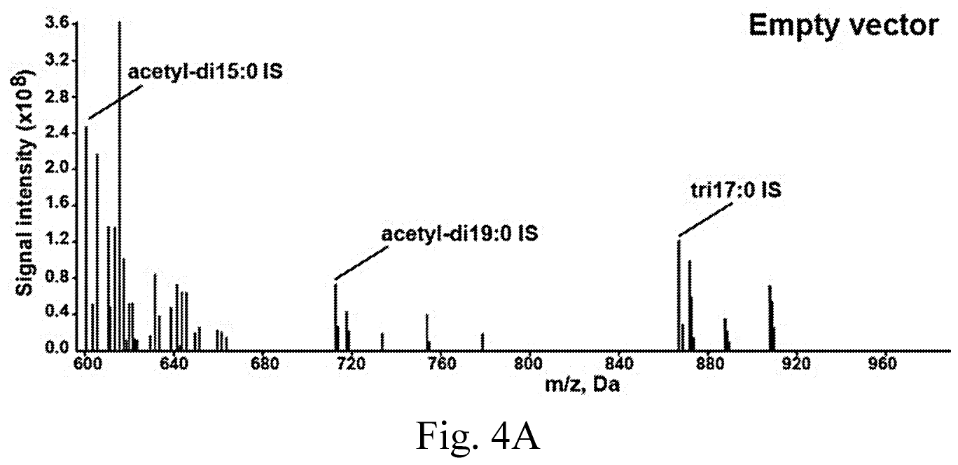

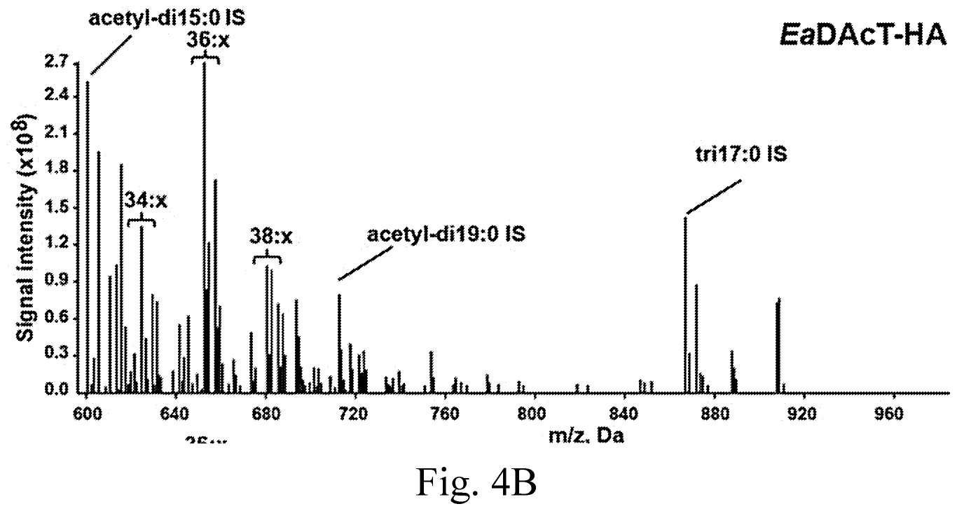

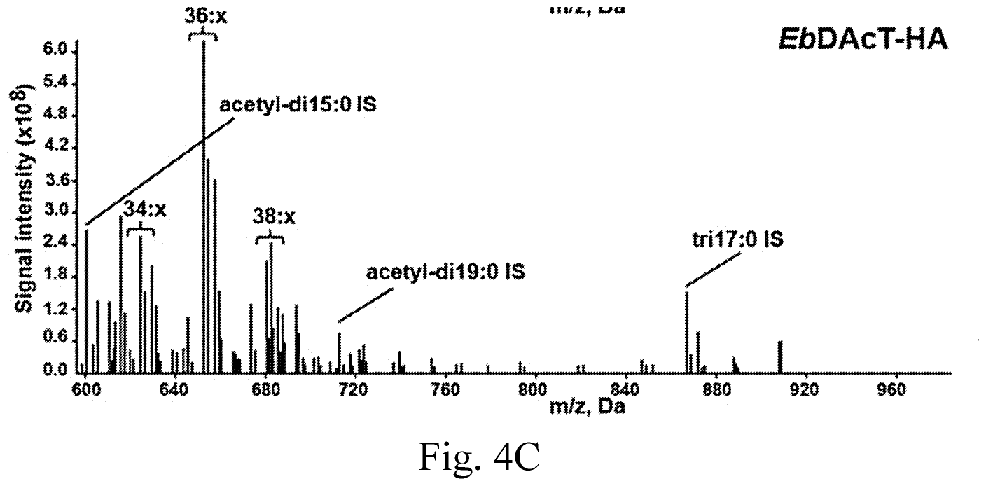

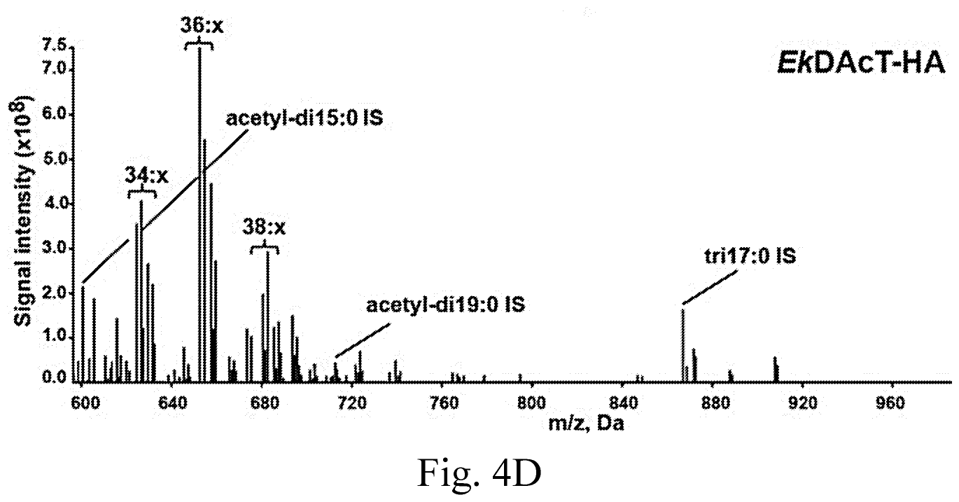

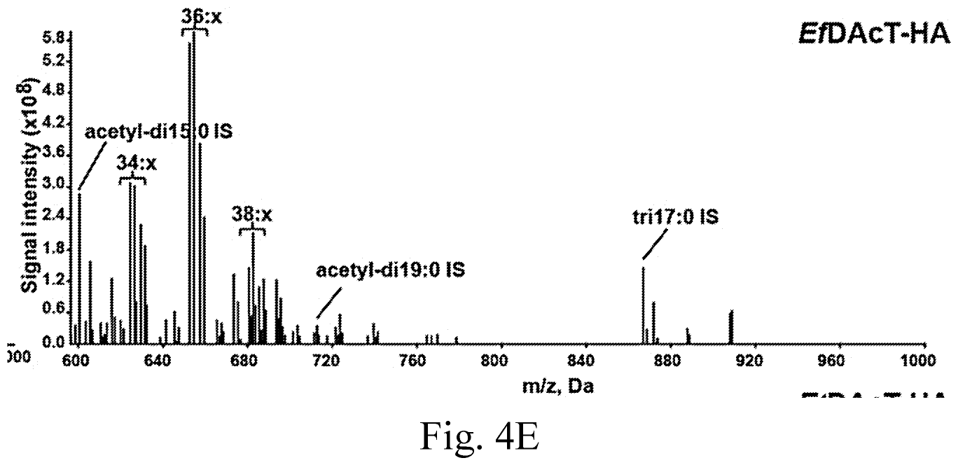

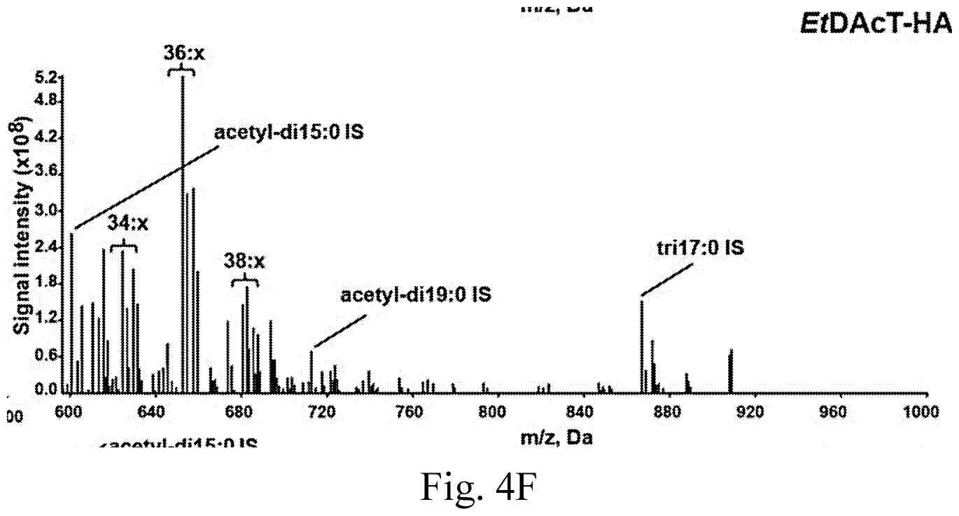

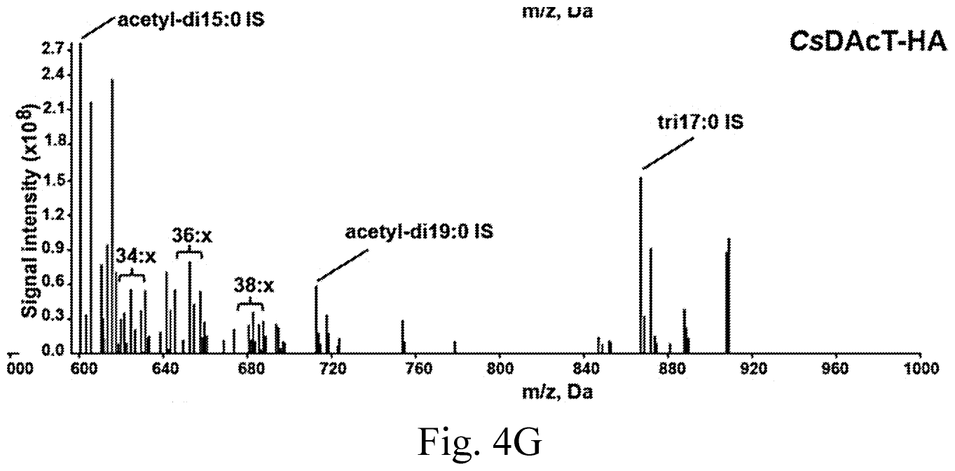

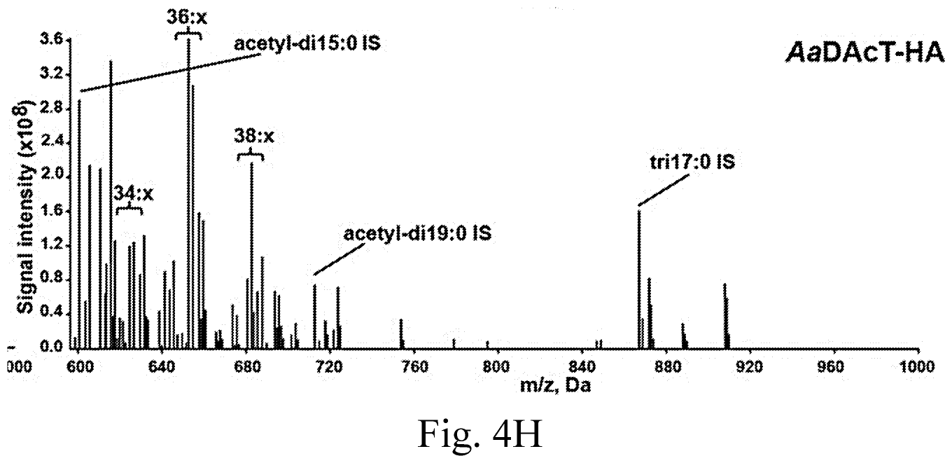

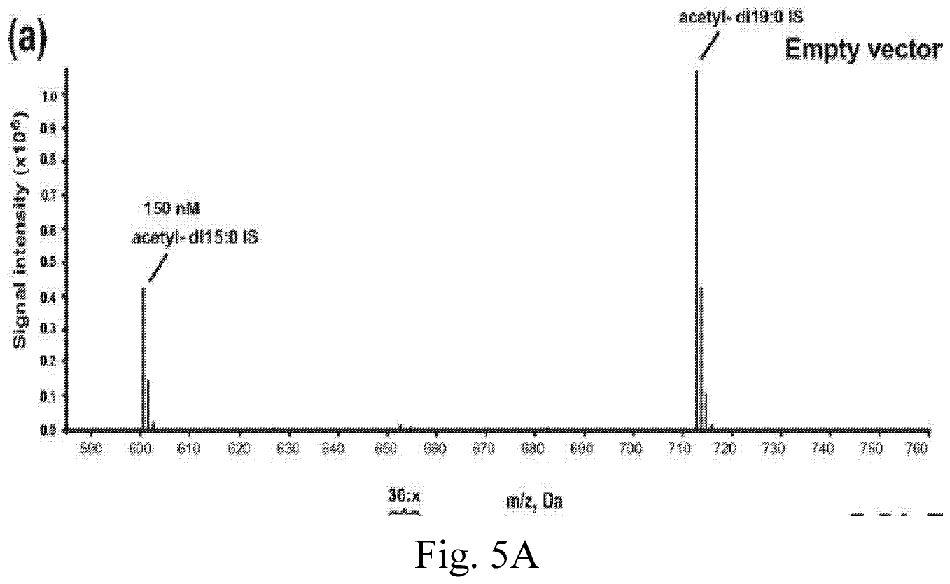

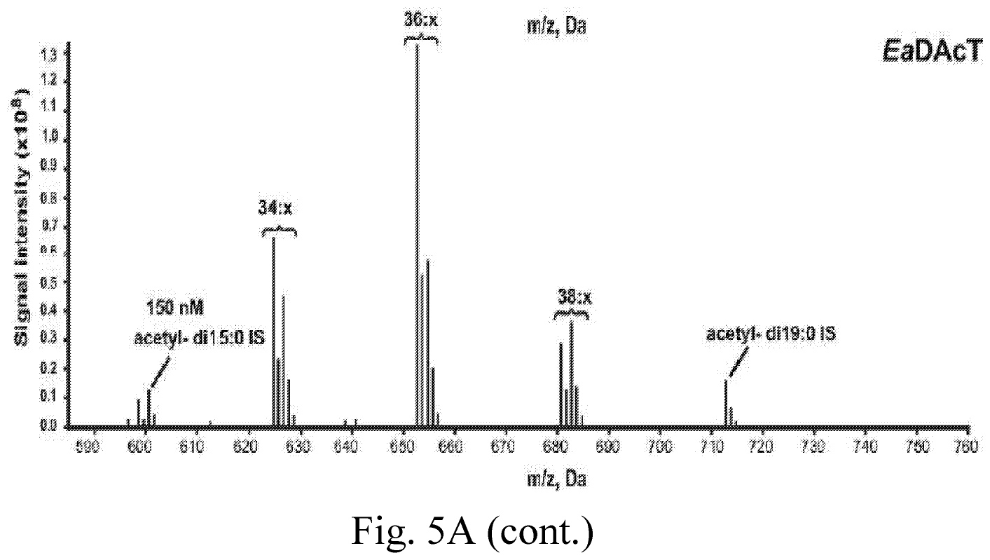

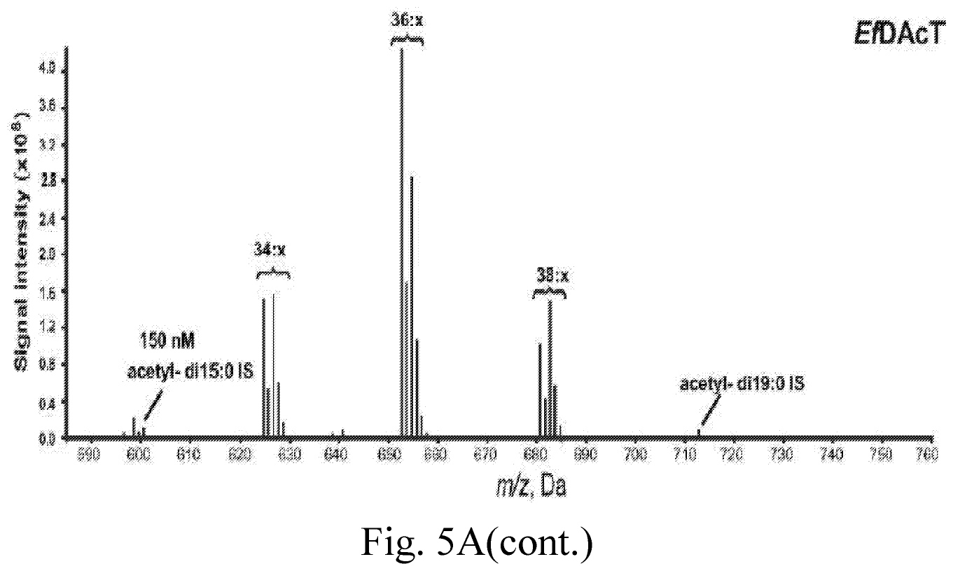

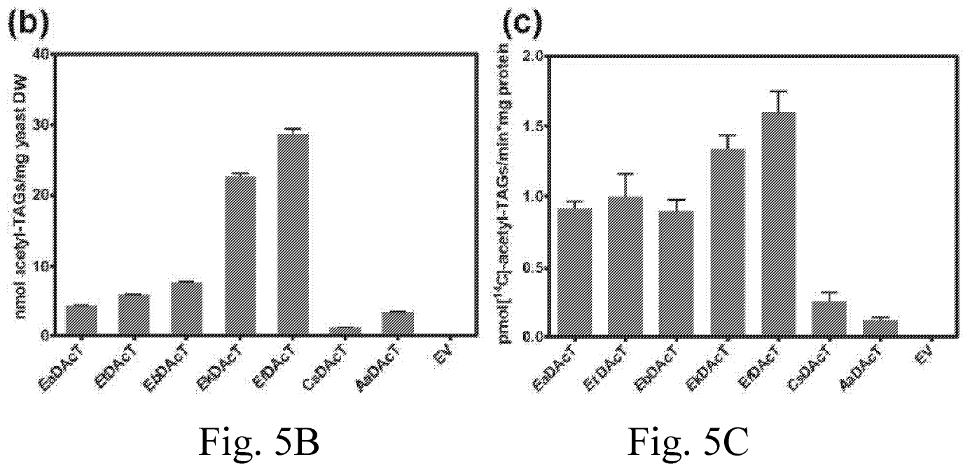

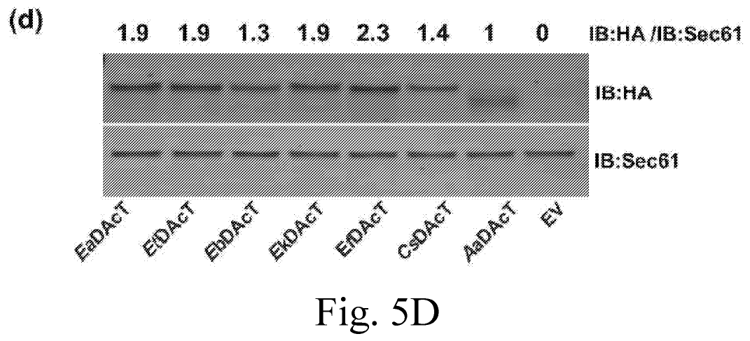

We identified the acetyltransferases responsible for the synthesis of acetyl-TAG in these species by isolating RNA from seed tissues and cloning expressed genes that were similar to EaDAcT. We then expressed these genes in H1246 yeast and analyzed lipid extracts to demonstrate that all enzymes were capable of synthesizing acetyl-TAG (FIGS. 4A-4H and FIG. 5A).

FIGS. 4A-4H show positive-ion electrospray ionization mass spectra of neutral lipid extracts from H1246 yeast expressing empty vector and HA-epitope tagged acetyltransferases. Peaks correspond to m/z values of the [M+NH4]+ adducts. The 3-acetyl-1,2-dinonadecanoyl-snglycerol (acetyl-di19:0) internal standard was added during lipid extraction and triheptadecanoin (tri17:0) and 3-acetyl-1,2-dipentadecanoyl-sn-glycerol (acetyl-di15:0) internal standards were added prior to ESI-MS. Peaks correspond to the m/z values of the [M+NH4]+ adducts of the intact acetyl-TAG molecules. The number of acyl carbons in each cluster of peaks is indicated; for the clarity, the number of double bonds (x) is not defined.

FIG. 5A shows representative mass spectra derived from the neutral loss of ammonium acetate from lipid extracts of yeast expressing the empty vector pYES2/CT, EaDAcT-HA or EfDAcT-HA. The internal standard 3-acetyl-1,2-dinonadecanoyl-sn-glycerol (acetyl-di19:0) was added during lipid extraction and 3-acetyl-1,2-dipentadecanoyl-sn-glycerol (acetyl-di15:0) was added during ESI-MS analysis. Peaks correspond to the m/z values of the [M+NH4]+ adducts of the intact acetyl-TAG molecules. The number of acyl carbons in each cluster of peaks is indicated; for the clarity, the number of double bonds (x) is not defined.

Similar to EaDAcT, no long chain TAG was detected in the lipids extracted from yeast expressing these new DAcT enzymes. These acetyltransferases unexpectedly varied in their ability to produce acetyl-TAG, despite their similarity to EaDAcT. For example, yeast expressing EfDAcT surprisingly accumulated the highest amount of acetyl-TAG with 28.7 nmoles/per mg DW, 6.7 times higher than the levels achieved through expression of EaDAcT (4.24 nmoles/mg DW; FIG. 5B). FIG. 5B shows acetyl-TAGs produced by yeast expressing different DAcTs quantified using ESI-MS. Values represent the mean±S.D. of acetyl-TAG content derived from three different ESI-MS analyses and are representative of at least two replicate cultures. Yeast expressing CsDAcT accumulated the lowest amount of acetyl-TAGs (1.1 nmoles/mg DW).

We isolated microsomes from yeast cells expressing the proteins and incubated them with [14C] acetyl-CoA to further confirm acetyltransferase activity of the successful enzymes. The formation of [¹⁴C] acetyl-TAG demonstrated that all DAcT enzymes possess acetyltransferase activity in vitro (FIG. 5C). In general, the in vivo accumulation of acetyl-TAG reflected the in vitro activity levels, with EfDAcT and EkDAcT showing higher activity compared to the other tested DAcT homologs (FIG. 5C). FIG. 5C shows yeast microsomes expressing different DAcT enzymes incubated with [14C]acetyl-CoA and resulting in vitro [14C]acetyl-TAG production quantified. Error bars indicate the standard deviation of 3 technical replicates. The results shown are representative of two independent experiments. Acetyltransferase protein levels present in the microsomes were quantified by immunoblotting against the HA epitope fused to the C-terminus of all proteins and normalizing to the abundance of Sec61 present in the yeast microsomes (FIG. 5D). FIG. 5D shows the abundance of different HA epitope-tagged DAcT enzymes examined by immunoblotting for the HA epitope or for Sec61 as a loading control. Numbers indicate the ratio of DAcT protein to Sec61 protein. In some cases, the variation in in vitro enzyme activity of the homologs was an unsurprising result of this investigation.

### DAcT Proteins are Distantly Related to DGAT1 Proteins

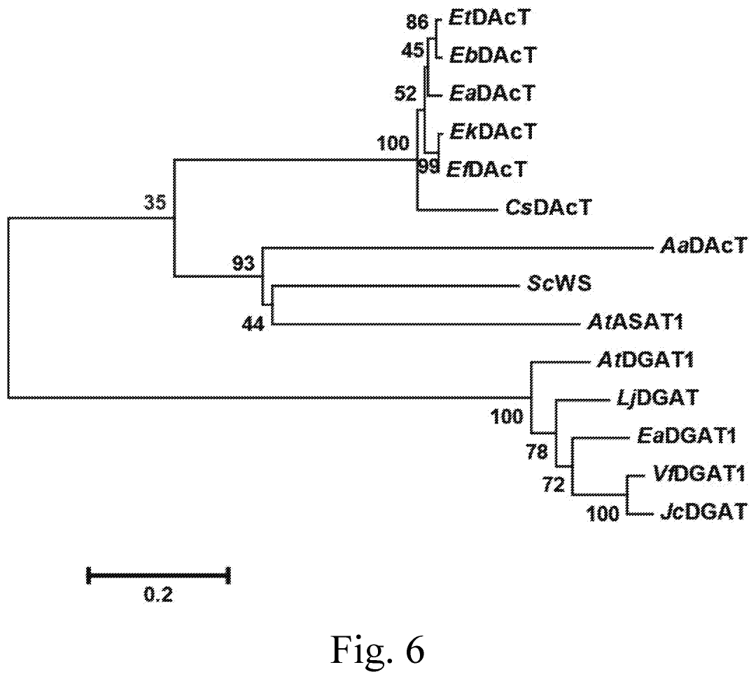

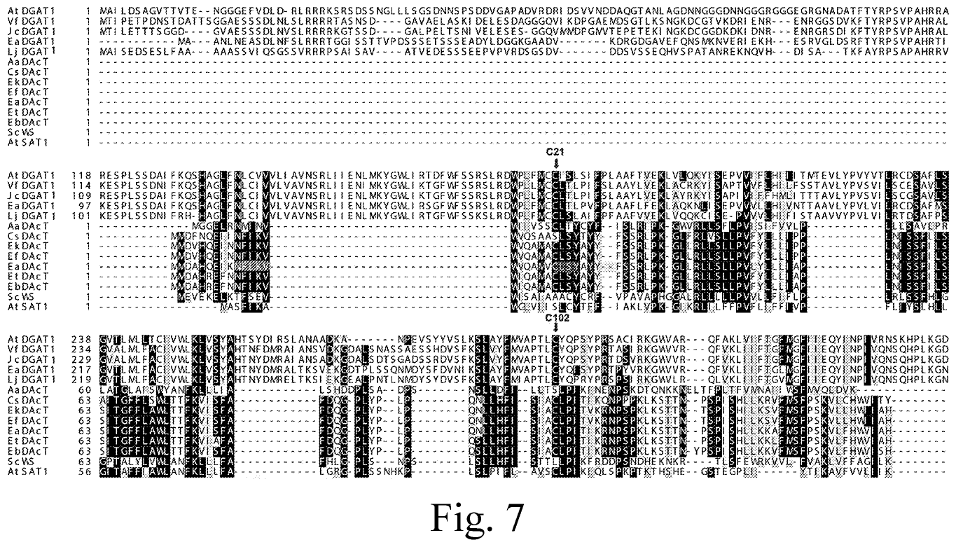

Consistent with their sequence similarity to EaDAcT, phylogenetic analysis indicated that the newly isolated DAcTs are more closely related to the Simmondsia chinensis (jojoba) wax synthase (ScWS) and Arabidopsis sterol acyltransferase (AtASAT1) than to DGAT1 proteins, which also acylate DAG FIG. 6). Evolution analyses were conducted in MEGA6. The evolutionary history was inferred using the neighbor-joining method and the evolutionary distances were computed using the Poisson correction method. Bootstrap values are shown in percentage at nodes. The tree (FIG. 6) is drawn to scale, with branch lengths in the same units as those of the evolutionary distances used to infer the phylogenetic tree. The 0.2 scale represents 20% change. Protein sequences used were DAcTs from Adonis aestivalis (Aa), Euonymus alatus (Ea), Euonymus atropurpureus (Et), Euonymus bungeanus (Eb), Euonymus kiautschovicus (Ek), Euonymus fortunei (Ef), Celastrus scandens (Cs), the Arabidopsis thaliana sterol O-acyltransferase 1 (AtASAT1) and the Simmondsia chinensis (jojoba) wax synthase (ScWS). All the newly isolated DAcTs cluster together, except for AaDAcT which like ScWS and AtASAT1 appears to have diverged away from the DAcT cluster.

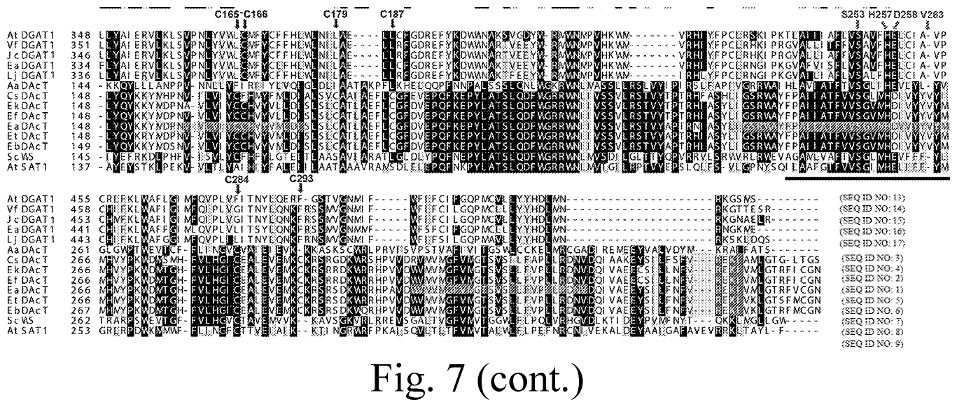

Protein sequences were aligned using Multiple Sequence Comparison by Log-Expectation (MUSCLE). (FIG. 7) Similar residues are shaded in grey and identical residues are indicated by white letters on a black background. The MBOAT signature region is underlined. Proposed transmembrane domains for EaDAcT are highlighted. The highlighted box indicates a predicted ER retention signal of DAcT proteins. Arrows indicate cysteine residues and residues where mutagenesis was conducted in the MBOAT region of EaDAcT. Sequences aligned were DAcT, DGAT, wax synthase (WS) and sterol O-acyltransferase 1 (ASAT1) proteins from Adonis aestivalis (Aa), Arabidopsis thaliana (At), Celastrus scandens (Cs), Euonymus alatus (Ea), Euonymus atropurpureus (Et), Euonymus bungeanus (Eb), Euonymus kiautshovicus (Ek), Euonymus fortunei (Ef), Jatropha curcas (Jc), Lotus japonicas (Lj), Simmondsia chinensis (Sc) and Vernicia fordii (Vf). A protein sequence alignment of these proteins revealed that the N-terminal region present in DGAT1 is absent from DAcTs, ScWS, and AtASAT1 (FIG. 7). In addition, the MBOAT homeodomain is highly conserved among the sequences with the invariant histidine embedded in a long stretch of hydrophobic residues (FIG. 7).

### Effects of Mutagenesis on Conserved Residues

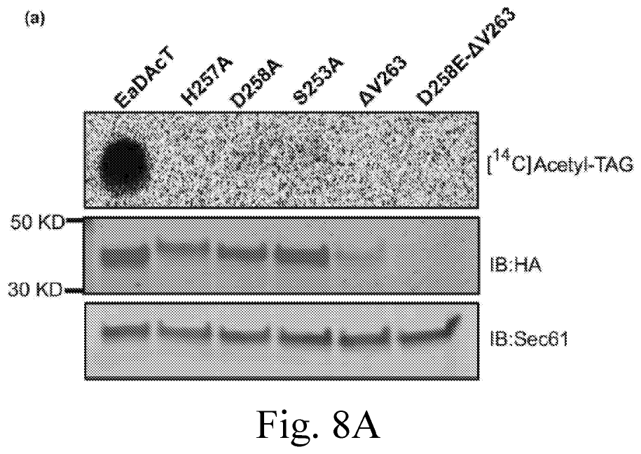

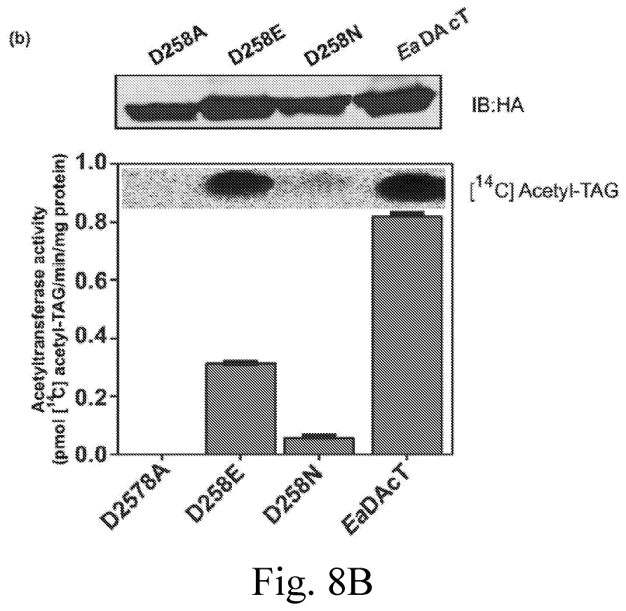

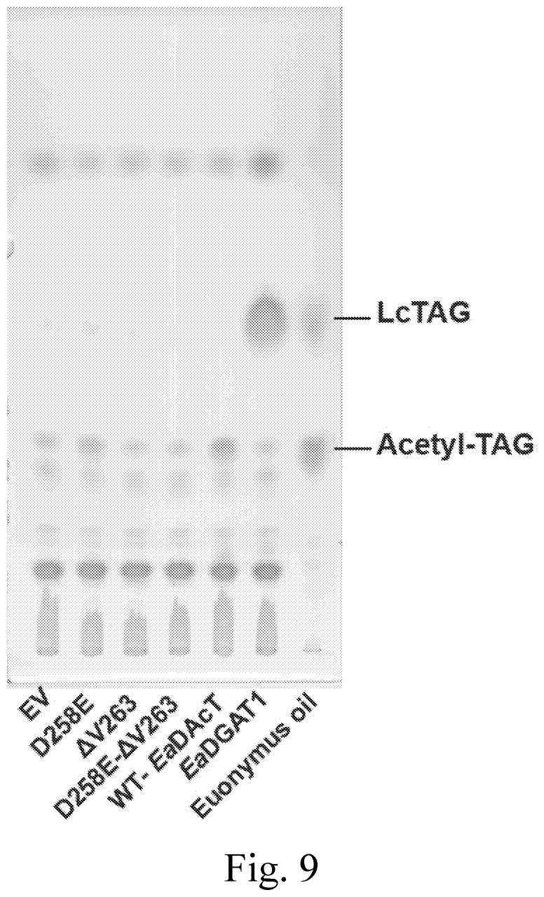

Comparisons of the MBOAT homology domains in the different acyltransferases identified four other residues of interest: S253, H257, D258 and V263. The first two of these, S253 and H257, are highly conserved in the MBOAT family. In our research, replacement of S253 and H257 with alanine abolished EaDAcT enzyme activity, revealing the essential role of these residues (FIG. 8A). FIG. 8A shows the effect of H257A, D258A, S253A, and ΔV263 mutations on acetyltransferase activity. Microsomes were isolated from yeast expressing different mutants and wild-type EaDAcT-HA. In vitro acetyltransferase activity was quantified by incubating with [14C]acetyl-CoA and detecting [14C]acetyl-TAG (upper panel). Protein levels of the EaDAcT mutants in the microsome fraction were detected by immunoblotting for the HA epitope (middle panel). Sec61 was used as a loading control (lower panel). The aspartate residue (D258) adjacent to the highly conserved H257 was intriguing given the conserved negative charge at this location and the potential role of such residues in a proposed catalytic triad serine-histidine-aspartate of ACAT1. Substitution of D258 with alanine also eliminated acetyltransferase activity (FIG. 8B). FIG. 8B shows the effect of different substitutions for D258 on EaDAcT activity. Microsomes were isolated from yeast expressing different mutants and wild-type EaDAcT-HA. In vitro acetyltransferase activity was quantified by incubating with [14C]acetyl-CoA and detecting [14C]acetyl-TAG (lower panel). Protein levels of the EaDAcT variants in the microsome fraction were detected by immunoblotting for the HA epitope (upper panel). Replacing D258 with asparagine to maintain a similar sized residue also greatly reduced in vitro activity, further demonstrating the importance of a negative charge at this position. Substituting D258 with glutamate to maintain a negative charge reduced acetyltransferase activity to 38% of the wild-type enzyme, indicating that the charge spacing is also important for maximal activity. This D258E mutation makes EaDAcT more similar to the sequences of acyltransferases that transfer long chain acyl-CoA; however the altered enzyme still does not possess the ability to synthesize 1cTAG (FIG. 9). FIG. 9 shows TLC separation of lipid extracts from H1246 yeast expressing empty vector, EaDAcTD258E, EaDAcT-ΔV263, EaDAcT-D258E-ΔV263, wild type EaDAcT and EaDGAT1 demonstrating no long chain TAG were produced by the mutant enzymes. Euonymus alatus oil was used as a TLC marker for acetyl-TAG and long chain TAG. Finally, the MBOAT homology domain in the acetyltransferases contains an additional amino acid, V263. Deletion of this residue to make the enzyme more like the acyltransferases that utilize long chain acyl-CoAs eliminated in vitro acetyltransferase activity and had a negative effect on the stability of the mutant protein (FIG. 8A). The ΔV263 mutant did not possess long chain acyltransferase activity (FIG. 9). We constructed the D258E ΔV263 double mutant to make EaDAcT even more similar to long chain acyltransferases; however, this mutant did not restore protein stability (FIG. 8A) or result in long chain acyltransferase activity (FIG. 9).

### Discussion

### Isolation of DAG Acetyltransferases from Other Acetyl-TAG Producing Species

In order to obtain more information about the residues important for the extreme acyl donor substrate specificity of EaDAcT, we isolated additional acetyltransferases from plant species that accumulate acetyl-TAG. All the newly identified DAcT enzymes were able to synthesize acetyl-TAG in vitro and in vivo, although some such as EfDAcT and EkDAcT possessed higher activity compared to others (FIG. 5C). This higher activity of EfDAcT and EkDAcT allowed yeast cells expressing these enzymes to accumulate considerably higher levels of acetyl-TAG during culture growth, compared to the other acetyltransferases (FIG. 5B), though other factors such as increased protein stability at different stages of culture growth still need to be determined. These results suggest that EfDAcT and EkDAcT might be useful enzymes for the biotechnological production of acetyl-TAG in transgenic oil seed crops or oleaginous yeast. Like EaDAcT, these new acetyltransferases are distantly related to DGAT1 proteins, despite the fact that both classes of enzymes are able to acylate DAG. Instead, the DAcTs are closely related to enzymes that acylate fatty alcohols and sterols.

### Residues Important for Acetyltransferase Activity

The acyltransferase mechanism of the MBOAT family is largely unknown due to the lack of structural information. One attractive hypothesis is the involvement of a serine-histidine-aspartate catalytic triad proposed to catalyze the acyltransferase reaction in ACAT1 and present in other enzymes involved in lipid metabolism, including lipases and acyltransferases. For some enzymes, glutamate functions in place of aspartate as the acidic group. Our mutagenesis data shows that S253, H257 and D258 are essential for the enzyme activity of EaDAcT. Consistent with these observations, S253 and H257 in EaDAcT are homologous to the serine and histidine residues in the putative catalytic triad of ACAT1. In many acyltransferases, including ACAT1, a glutamate replaces D258 but the role of this residue was not tested in ACAT1. Instead, an aspartic residue corresponding to E197 in EaDAcT was proposed to participate in ACAT1's catalytic triad. However, an acidic group is not present in this position in AtASAT1, casting some doubt on whether this residue participates in a conserved catalytic mechanism. Instead, the low activity of D258A and D258N and the high conservation of aspartate or glutamate in analogous positions in acyltransferases suggest the importance of a negative charge at D258. Further, the D258E mutants which retained that negative charge possessed approximately 50% lower activity, indicating that the spacing of a negative charge is important for activity in EaDAcT. Consistent with this result, AaDAcT which is more similar to long-chain utilizing acyltransferases, possesses a glutamate instead of aspartate at position 258, possibly explaining this enzyme's weak acetyltransferase activity.

Another unique and highly conserved residue among the DAcTs is the additional valine at position 263 in EaDAcT. Deletion of this valine residue significantly reduces the stability of EaDAcT. This result is consistent with the low stability of AaDAcT when expressed in yeast and suggests that this valine residue is important for DAcT protein stability. However, further mutagenesis on AaDAcT is needed to justify this assumption.

The role of cysteine in the enzymatic activity of MBOATs has not been studied intensively. A study on human ACAT1 showed that although thiol-specific modification severely inhibits the enzyme activity, the cysteine-free enzyme still possesses 40% of the activity levels of the wild type protein. In contrast, the enzyme activity of EaDAcT was greatly reduced when all nine endogenous cysteine residues of the protein were mutated to alanine. The two residues most important for activity, C187 and C293, are highly conserved in all DAcT orthologs except for AaDAcT. It is unlikely that these two cysteines together form a structurally important disulfide bond as they are located on opposite sides of the lipid bilayer membrane.

### Conclusion

In summary, the project involved sequencing multiple species that produce acetyl-TAGs to develop the acetyltransferase enzymes. In only two species did enzymes show unexpected increased activity compared to EaDAcT (e.g., EfDAcT, EkDAcT). In most cases, the enzymes possessed the same or lower activity compared to EaDAcT. Further, in some species, the most similar enzymes to EaDAcT did not possess acetyltransferase activity. These enzymes are referred to as SaWS (from Sorbus aucuparia), AaWS (from Adonis aestivalis) and AqWS (from Akebia quinate) based on the fact that they appear to function as wax synthases. The lack of acetyltransferase activity in SaWS, AaWS and AqWS provides additional evidence that merely identifying close homologs of EaDAcT is insufficient to find acceptable acetyltransferases.

Both EfDAcT and EkDAcT had unexpected increased activity as compared to EaDAcT, despite the fact that EfDAcT has 94% (341/363 residues) sequence identity to EaDAcT. Sequence analysis reveals that a particular conserved region may be responsible for acetyltransferase activity in the various enzymes. In addition, the data reveals similar enzymes that possess different activities, despite otherwise similar sequences. In some cases, enzymes from other species unexpectedly lacked acetyltransferase activity, when otherwise similar to EaDAcT.

### Example II

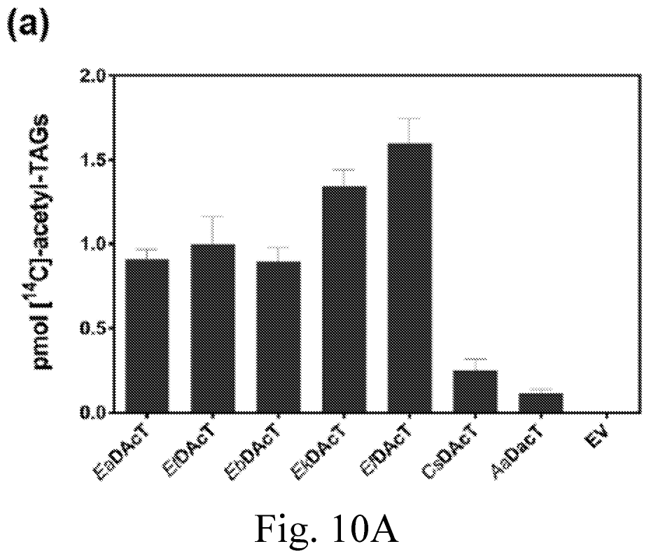

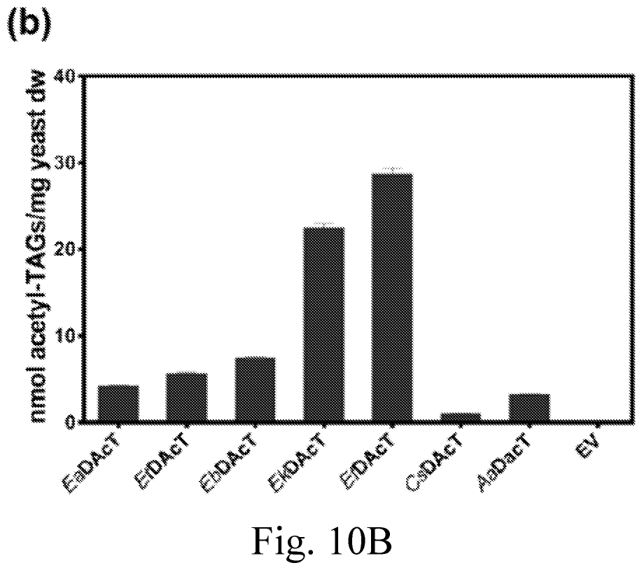

In this experiment, acyltransferases from species other than Euonymus alatus that synthesize acetyl-TAGs were further examined. As described above, two of these enzymes, EfDAcT and EkDAcT (derived from Euonymus fortunei and Euonymus kauschovicus, respectively), when expressed in yeast possessed unexpectedly higher in vitro enzyme activity than EaDAcT and other similar sequences (FIG. 10A). Further, the yeast expressing these two enzymes is capable of accumulating approximately 6-fold more acetyl-TAGs compared to EaDAcT and other similar sequences (FIG. 10B).

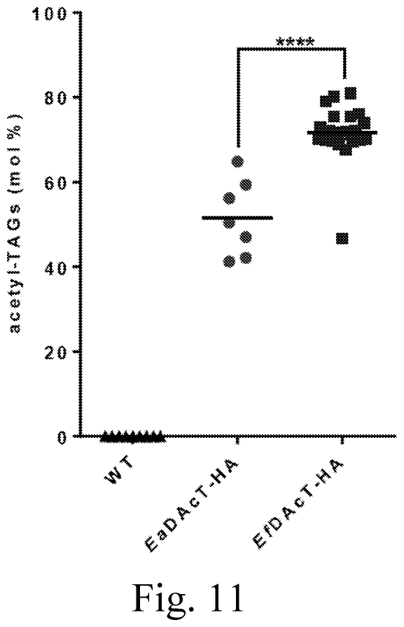

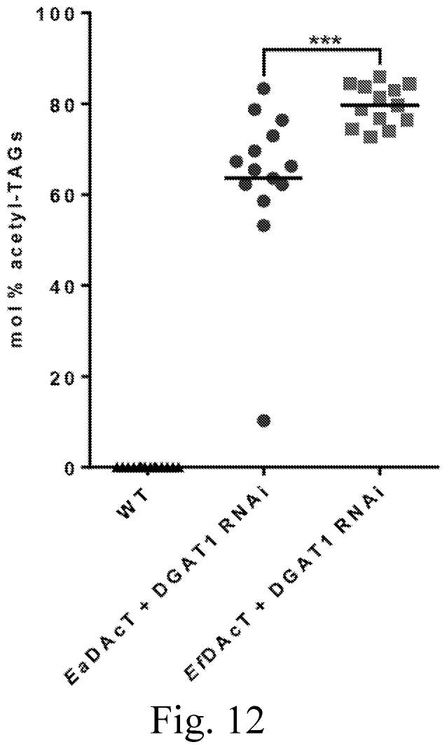

Importantly, the improved ability of EfDAcT to synthesize acetyl-TAGs is evident in experiments with transgenic plants. For example, expression of EfDAcT in Camelina sativa resulted in higher levels of acetyl-TAGs in transgenic seeds compared to when EaDAcT was used (FIG. 11). Further, when combined with the suppression of the competing DGAT1 enzyme, acetyl-TAG levels as high as 90 mol % were achieved in the best transgenic lines (FIG. 12), surpassing the previously published levels in the best EaDAcT lines. EfDAcT is therefore a superior enzyme to EaDAcT with regard to the production of acetyl-TAGs.

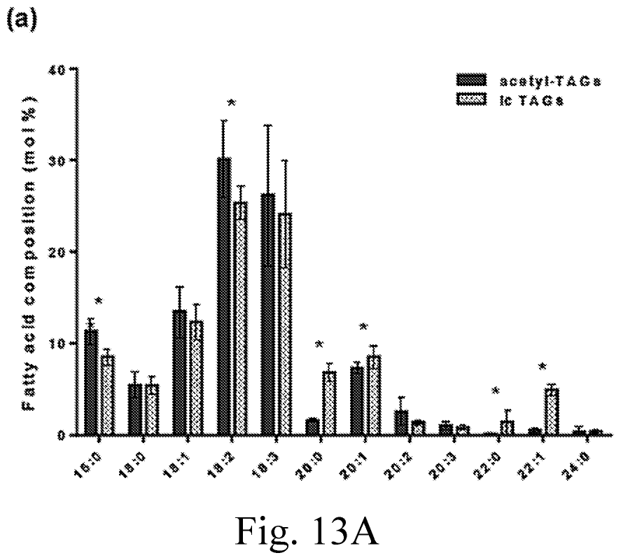

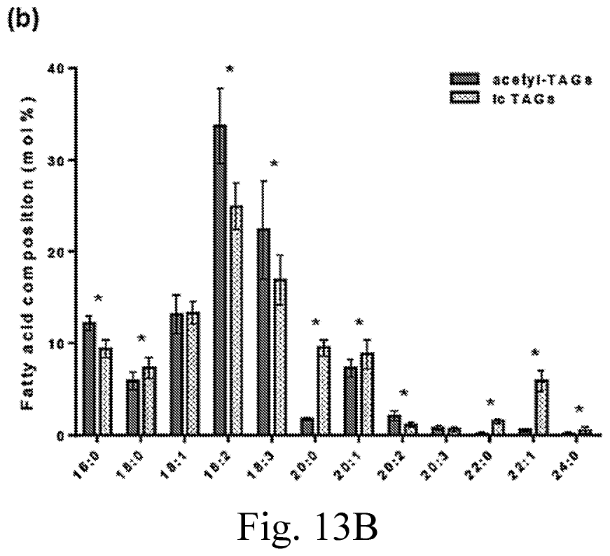

FIG. 13 shows the fatty acid composition of Camelina sativa plants expressing EaDAcT+DGAT1-RNAi or EfDAcT+DGAT1-RNAi, showing differences between the two. For example, acetyl-TAGs from EfDAcT possess higher levels of the 18:2 fatty acid. At this point it is unclear the reason for the difference (multiple explanations possible) but it does provide additional evidence that the enzymes are different.

The project involved sequencing multiple species that produce acetyl-TAGs to develop the acetyltransferase enzymes. Only in two species were enzymes with unexpected increased activity than EaDAcT identified (e.g., EfDAcT, EkDAcT). In most cases, the enzymes possessed the same or lower activity compared to EaDAcT. Further, in some species, the most similar enzymes to EaDAcT did not possess acetyltransferase activity. These enzymes are referred to as SaWS, AaWS and AqWS (based on the fact that they appear to function as wax synthases). The lack of acetyltransferase activity in SaWS, AaWS and AqWS provides additional evidence that just looking for close homologs of EaDAcT is not enough to find acetyltransferases.

Both EfDAcT and EkDAcT had unexpected increased activity as compared to EaDAcT, despite the fact that EfDAcT has 94% (341/363 residues) sequence identity to EaDAcT.

Sequence analysis reveals that a particular conserved region may be responsible for acetyltransferase activity in the various enzymes. In addition, the data reveals similar enzymes that possess different activities, despite otherwise similar sequences. In some cases, enzymes from other species unexpectedly lacked acetyltransferase activity, when otherwise similar to EaDAcT.

In the sequence alignment shown in FIGS. 2A and 2B, a conserved region of interest is highlighted. The histidine residue is seen to be highly conserved among all species. The conserved region that follows appears responsible for high acetyltransferase activity: HDXVYYVY, where X is V or I (SEQ ID NO:40). Thus, aspects of the invention involve enzymes capable of synthesizing acetyl-TAGs that comprise at least this conserved sequence. Based upon mutagenesis data gathered, modifications in this region result in a loss or reduction of acetyltransferase activity.

### Example III

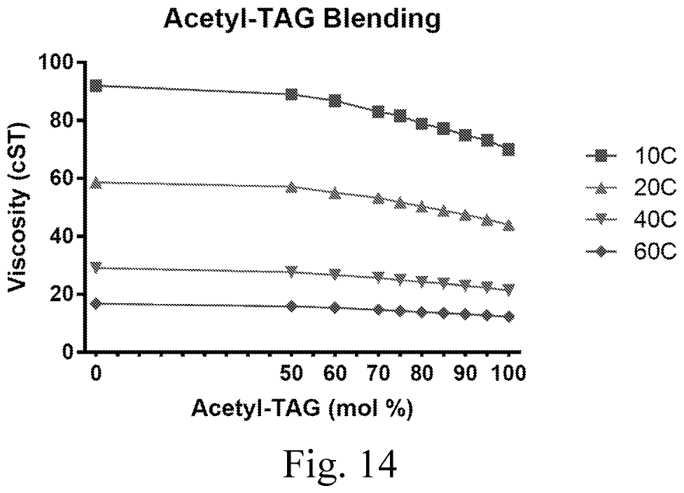

Acetyl-TAG/oil blends having various levels of acetyl-TAG were prepared. The viscosities of these blends were determined at different temperatures, and the results are presented in FIG. 14.

### Example IV

In diesel engines, compression of the air in-cylinder results in hot dense gases present at the time of fuel injection. The fuel is sprayed into the engine cylinders at high pressure by fuel injectors, resulting in atomization of the fuel. Entrainment of the hot dense in-cylinder gases evaporates the fuel droplets and results in ignition of fuel. High molecular weight alternative fuels have been noted to exhibit longer ignition delay periods resulting in late combustion in the expansion stroke. For example, Jatropha oil showed longer ignition delays compared to diesel. This was attributed to the higher viscosity of the vegetable oil compared to diesel, which led to poor atomization and mixture preparation with air during the ignition delay period. Extensive delays between injection and ignition can lead to unacceptable rates of cylinder pressure rise as too much fuel takes part in combustion or to misfire.

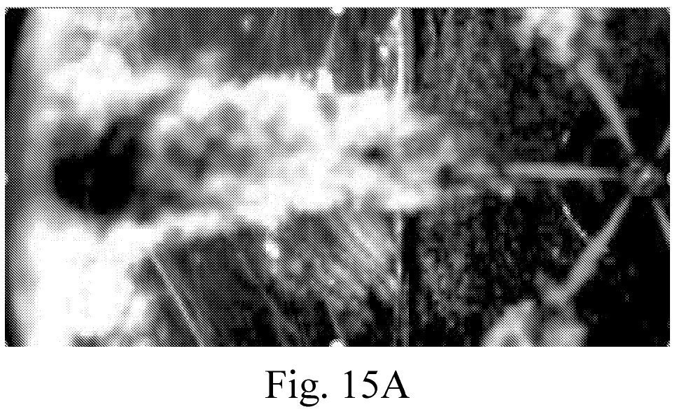

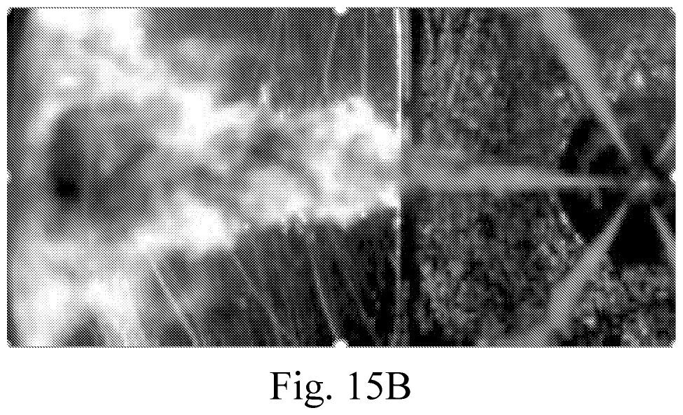

The combustion performance of acetyl-TAGs in accordance with embodiments of the present invention were tested for combustion performance in a diesel engine (31.3 kg/m³). The performance was compared to standard diesel fuel combustion performance. The results are summarized below and in FIGS. 15A and 15B.
In-cylinder conditions at start of injection (−5 CAD)
   Pᵢₙⱼ=8050 kPa (80.5 bar)
   Tᵢₙⱼ=895 K
   ρᵢₙⱼ=31.3 kg/m³
Ignition delay (ID)
   Acetyl TAG=417 μs
   Diesel=451 μs
Acetyl TAG vs. Diesel
   Shorter ID
   Very closely matched rate of heat release and pressure

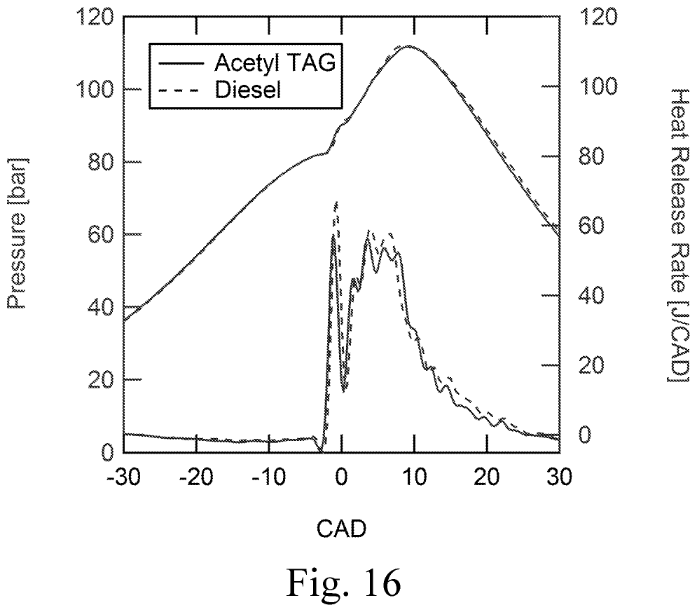

In contrast to typical vegetable oils, which exhibit longer ignition delays, analysis of the combustion properties of acetyl-TAGs revealed that these molecules possess a slightly shorter ignition delay (417 μs) compared diesel (450 μs) at high density (˜30 kg/m³) and moderate temperature (˜900 K) in-cylinder conditions. Consequently the rates of heat release and cylinder pressure for the combustion of acetyl-TAGs are very similar to that of diesel (FIG. 16). As shown in FIGS. 15A and 15B, acetyl-TAG and diesel show more similar behavior at high ambient density, and acetyl-TAG exhibits combustion recession (flashback) at EOI.

These improvements in combustion properties relative to typical vegetable oils could be attributed to the reduction in viscosity and increased volatility of acetyl-TAG, which results in improved atomization and evaporation of the fuel.

1. A vector comprising an isolated nucleic acid sequence operably linked to a heterologous promoter, wherein the nucleic acid encodes a short chain acyl-CoA diacylglycerol acyltransferase plant protein that is at least 95% identical to SEQ ID NO:1 or SEQ ID NO:2.

2.-4. (canceled)

5. A host cell comprising the vector of claim 1.

6.-7. (canceled)

8. The host cell of claim 5, wherein said host cell is a plant cell from a Jatropha plant, an oil crop plant, a palm oil plant, an alga, a Brassica plant, a Brassicaceae plant, an Arabidopsis plant, a Camelina plant, a crambe plant, or a Camelina sativa plant.

9.-11. (canceled)

12. The host cell of claim 5, wherein said host cell is a fungus cell.

13.-18. (canceled)

19. A method comprising:
providing an isolated nucleic acid sequence encoding a protein that is at least 95% identical to SEQ ID NO:1 or SEQ ID NO:2, and a host cell;
transforming said host cell with said isolated nucleic acid sequence such that said nucleic acid expresses said protein in said transformed host cell; and
isolating an acetyltriacylglycerol from said transformed host cell.

20. The method of claim 19, wherein said acetyltriacylglycerol is a 3-acetyl-1,2-diacyl-sn-glycerol.

21. The method of claim 19, wherein said isolating comprises lipid extraction.

22. The method of claim 19, wherein said transformed host cell further comprises a heterologous gene and expresses said heterologous gene under conditions for increasing a substrate for said protein.

23. The method of claim 22, wherein said heterologous gene encodes a fatty acid elongase 1 mutant protein, an ATP-citrate lyase enzyme, or an acyl-ACP thioesterase (FatB2) protein.

24. The method of claim 22, wherein expression of said heterologous gene reduces long chain fatty acid synthesis.

25.-26. (canceled)

27. The method of claim 19, wherein said transformed host cell further comprises an inhibitory heterologous nucleic acid capable of interfering with the production of a long-chain-triacylglycerol molecule for increasing amounts of isolated acetyltriacylglycerol.

28. The method of claim 27, wherein said inhibitory nucleic acid is selected from the group consisting of a diacylglycerol acyltransferase 1 gene, diacylglycerol acyltransferase 2 gene, and phospholipid:diacylglycerol acyltransferase gene.

29. The method of claim 27, wherein said inhibitory nucleic acid is an siRNA.

30. The method of claim 27, wherein said production of long chain-triacylglycerol molecules is reduced.

31. The method of claim 19, wherein said transformed host cell has low long chain-triacylglycerol production.

32. The method of claim 31, wherein said transformed host cell expresses a mutant fatty acid elongase 1 gene resulting in low long chain-triacylglycerol production.

33. The method of claim 19 comprising:
providing a transgenic plant part comprising said transformed host cell;
growing said transgenic plant part under conditions such that said nucleic acid expresses said protein wherein acetyltriacylglycerol production is increased in said transgenic plant part; and
isolating acetyltriacylglycerol from said transgenic plant part.

34. The method of claim 33, wherein said acetyltriacylglycerol is 3-acetyl-1,2-diacyl-sn-glycerol.

35. The method of claim 33, wherein said plant part is selected from a seed, aril, stem, leaf, tubers, mesocarp, pericarp, exocarp, cell wall, and frond.

36.-46. (canceled)

47. The method of claim 19, further comprising:
applying oil isolated from said host cell comprising said acetyltriacylglycerol as a lubricant, biofuel, spray coating, food oil, in food processing, or in thermoplastic polymer products.

48.-59. (canceled)


SEQUENCE LISTING (40 sequences)

SEQ ID NO: 1 - protein, 363 aa, Euonymus fortunei
MMDVHQEINNFIKVWVQAMACLSYAYYFSSRLPKGLLRLLSLLPVFYLLLIPPLNISSFILSSITGFFLAWLTTFKVISFAFDQGPLYPLPQNLLHFISIACLPITVKRNPSPKLKSTTNTSPISHILKSVFMSFPSKVLLHWIIAHLYQYKKYMDPNVVLVIYCCHVYVLLDISLSLCATLAEFLCGFDVEPQFKEPYLATSLQDFWGRRWNIIVSSVLRSTVYTPTRHIASYLIGSRWAYFPAIIATFVVSGVMHDIVYYVYMMHVYPKWDMTGHFVLHGICEALEVEMKCKRSRRDKWRRHPVVDWVMVMGFVMGTSVSLLFVPLLRDNVDQIVAEECSILLNFVREKIVMLGTRFICGN

SEQ ID NO: 2 - protein, 363 aa, Euonymus kiautschovicus
MMDVHQEINNFIKVWVQAMACLSYAYYFSSRLPKGLLRLLSLLPVFYLLLIPPLNISSFILSSITGFFLAWLTTFKVISFAFDQGPLYPLPQNLLHFISIACLPITVKRNPSPKLKSTTNTSPISHILKSVFMSFPSKVLLHWIIAHLYQYKKYMDPNAVLVIYCCHVYVLLDISLSLCATLAEFLCGFDVEPQFKEPYLATSLQDFWGRRWNIIVSSVLRSTVYTPTRHIASYLIGSRWAYFPAIIATFVVSGVMHDIVYYVYMMHVYPKWDMTGHFVLHGICEALEVEMKCKRSRRDKWRRHPVVDWVMVMGFVMGTSVSLLFVPLLRDNVDQIVAEECSILLNFVREKIVMLGTRFICGN

SEQ ID NO: 3 - protein, 350 aa, Adonis aestivalis
MGGELRNMINVWIIVSSCLTYCYFISLRIPKGWVRLLSFLPVISIFVVLPLLLSAVLPRLATGLAISWYANFKLLLIALSHDDPLSADPSNSLLDFILLTSLPIKINENPSKDTQNKKNELTFPLTFVMNAIIWSFMVQLDVKKKQYLLLANPPVNNLLYFIRIIYLVQIGLDLIAATAKPFLKHELQQPFNNPALSSSLQNLWGKRWNLMASSLLRSLVYIPIRSLFAPIVGRRWAIHLAVIATFTVSGLIHEVLYLYVGLGWPTWEVTCFFLINGVCVAIEIEVKKKASKSCWRLPRVISVPSTVAFIVITGSWLCCKELMRCGADIREMEEYVALVDYMKRAIFATS

SEQ ID NO: 4 - protein, 362 aa, Celastrus scandens
MMDFNQEIINFIKVWVQSAASLSYTYYFSSRLPKGLFRLVSLLPVFYLLLIPPLNISSFLLSAITGFFLSWLTTFKVISFAFDQGPLYPLPQNLLHFISIACLPITIKQNPPPKLKSTTNPSPISHLLKRVFMSFPSKVLCHWVITYLYQYKKYMHPNVILVIYCFHMYFMLDIALSVCAAIAEFLCGFEVEPQFKEPYLATSLQDFWGRRWNLIVSSVLRSTVYTPTRHFASHLIGSRWAYFPAIIATFVVSGLMHDIVYYVYIMHVYPKWDMSMHFVLHGICEALEVEMKCKRSRGDKWRSHPVVDRVMVMGFVMGTSVSILFAPLLRDNVDQIAAKEYSILFSFVREKIAMLGTGLTGS

SEQ ID NO: 5 - protein, 363 aa, Euonymus alatus
MMDAHQEIKNFIKVWVQAMVCLSYAYYFSSRLPKGLLRLLSLLPVLYLLLIAPLNISSFILSSITGFFLAWLTTFKVISFAFDQGPLYPLPQNLLHFISIACLPITIKRNPSPKLKSTTNPSPISHLLKKAFMSFPSKVLFHWVIAHLYQYKKYMDPNVVLVIYCCHVYVMLDISLSLCATLAEFLCGFDVEPQFKEPYLATSLQDFWGRRWNIIVSSVLRSTVYAPTRNIASYLIGSRWAYFPAIIATFVVSGVMHDVVYYVYMMHMYPKWDMTGHFVLHGICEALEVEMKCKRSRSDKWRRHPAVDWVMVMGFVMGTSVSLLFVPLLRDNVDQIVAEEYSILFNFVREKIVMLGTRFVCGN

SEQ ID NO: 6 - protein, 363 aa, Euonymus atropurpureus
MMDAHREFNNFIKVWIQAMACLSYAYYFSSRLPKGLLRLLSLLPVFYLLLIAPLNISSFILSSITGFFLAWLTTFKVIAFAFDQGPLYPLPQSLLHFISIACLPITIKRNPSPKLKSTTNPSPISHLLKKLFMSFPSKVLFHWVIAHLYQYKRYMDPNVVLVIYCCHVYVMLDISLSLCATLAEFLCGFDVEPQFKEPYLATSLQDFWGRRWNIIVSSVLRSTVYAPTRHIASYLIGSRWAYFPAIIATFVVSGVMHDIVYYVYMMHMYPKWDMTGHFVLHGICEALEVEMKCKRSRSDKWRRHPVVDWVMVMGFVMGTSVSLLFVPLLRDNVDQIVAEEYSILFNFVREKIVKLGTSFMCGN

SEQ ID NO: 7 - protein, 364 aa, Euonymus bungeanus
MMDAHREFNNFIKVWIQAMACLSYAYYFSSRLPKGLLRLLSLLPVFYLLLIAPLNISSFILSSITGFFLAWLTTFKVISFAFDQGPLYPLPQSLLHFISIACLPITIKRNPSPKLKSTTNYPSPISHLLKKVFMSFPSKVLFHWVISHLYQYKKYMDSNVVLVIYCCHVYVMLDISLSLCATLAEFLCGFDVEPQFKEPYLATSLQDFWGRRWNIIVSSVLRSTVYAPTRHIASYLIGSRWAYFPAIIATFVVSGVMHDIVYYVYMMHMYPKWDMTGHFVLHGICEALEVEMKCKRSRSDKWQRHPVVDWVMVMGFVMGTSVSLLFVPLLRDNVDQIVAEEYSILFNFVREKIVKLGTRFMCGN

SEQ ID NO: 8 - protein, 352 aa, Simmondsia chinensis
MEVEKELKTFSEVWISAIAAACYCRFVPAVAPHGGALRLLLLLPVVLLFIFLPLRLSSFHLGGPTALYLVWLANFKLLLFAFHLGPLSNPSLSLLHFISTTLLPIKFRDDPSNDHEKNKRTLSFEWRKVVLFVAKLVFFAGILKIYEFRKDLPHFVISVLYCFHFYLGTEITLAASAVIARATLGLDLYPQFNEPYLATSLQDFWGRRWNLMVSDILGLTTYQPVRRVLSRWVRLRWEVAGAMLVAFTVSGLMHEVFFFYLTRARPSWEVTGFFVLHGVCTAVEMVVKKAVSGKVRLRREVSGALTVGFVMVTGGWLFLPQLVRHGVDLKTIDEYPVMFNYTQKKLMGLLGW

SEQ ID NO: 9 - protein, 345 aa, Simmondsia chinensis
MASFIKAWGLVIISLCYTFFIAKLVPKGIKRLILFFPVFLIFFIVPFLIYSLHLLGITAFFIAWLANFKLLLFALGRGPLSSNHKPLSLPIFLAVSCLPIKIQLSPKPTKTHSHEGSTEGPLIYTIKAVFVVLIIKAYEYSTKLPEKVVLTLYAIHIYFALEIILAATAAAVRAMSDLELEPQFNKPYLATSLQDFWGRRWNLMVTGILRPTVYEPSLQLFSVLGPNYSQILAAFGTFVVSGIMHELIFFYMGRLRPDWKMMWFFLINGFCTTVEIAIKKTINGRWRFPKAISQVLTLTFVMVTALWLFLPEFNRCNIVEKALDEYAAIGAFAVEVRRKLTAYLF

SEQ ID NO: 10 - protein, 367 aa, Sorbus aucuparia
MEDEIYNFIKVCFLANISLCYCFYISSRIPKGILRLISLLPILYLFILLPLNLHSFHLGGPTTFLLVWLATFKLLLFSFDHGPLSSPPPLTLFQFISLAILPIKIKQNPPQKSPPNQNPDHKIPPNPTKSENPSYPIGKKSTLFAVKALLLALVVGSYDYRQHLHPYVILALYCCHLYLGLEILLPLFATPARAVFGFELEPHSNEPYLSTSLQDFWGRRWNLMVTKILRPTTYYPVRNVFTPVVGPRLARFLAMMSTFAVSGLMHEVIYYYLARVPPTWEVTWFFVLHGVCVAIEVEVKKAVADRWRLKRVVSGPLTLLFLAVTANWLFFPQLLRNGVDVKAINEYPVMVDFVKSHLPLNFYWWKS

SEQ ID NO: 11 - protein, 347 aa, Adonis aestivalis
MKGVNELKSLIQVWATVFASLTLCHLLIRRIPQGITRLIFLLPIITLFTILPLKLSSIHLTGITSFFVTWLCNFKLFLFAFNQGPLSPSISLPHFICIACLPIQIQQEKQRNPSHQTTQTGLKSPLNYSIKALLLALVIKVYDYKPFLHHNLILTLYCFHVYLALEVVLAISAVPARAIFGFEIEPQFDDPYLSTSLQDFWGKRWNLMVSRILRPTVYNPIRYICTPTFGKRWALVMGLWSTFLVSGLMHELIYFYLTRARPTWEVTWFFVLHGVCTSVEIIGKNALGGRWGLHPLLATILTVGFVAITGFWLFFAQIVRCEVDVKTIGEYRSLVEFLKHVLFSQKR

SEQ ID NO: 12 - protein, 353 aa, Akebia quinata
MEGEINGLIKVFLSILTSLSYCYFLVSQIPKGNIRLISLLPVFYLFFLLPFSLSSVHFRSIIAFSISWLANFKLLLFAFDQGPLSSPISLLRFICICSLPLKIKPPNPSPQLLLSKPTPKSPFQTPIKLLIFVLLIRIHDYKQQIHPKVMCVLYGLIVYFFAETVLSASAILARVLLDLEVEPQFDEPYLSTSLQDFWGRRWNLVVTSILRPTVYVPVRCISTRMFGGRSRRWAPLPALLATFLVSGLMHELLYFYITRVRPTWEVTWFFVLHGICLAIEIEVKKAVSGRWRLHPVVSVPLAVGFVAVTGFWLFFPQLLRNGVDDRVTGEYRSLIEFVKEKWVWIIHGSSSPL

SEQ ID NO: 13 - protein, 520 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMS

SEQ ID NO: 14 - protein, 526 aa, Vernicia fordii
MTIPETPDNSTDATTSGGAESSSDLNLSLRRRRTASNSDGAVAELASKIDELESDAGGGQVIKDPGAEMDSGTLKSNGKDCGTVKDRIENRENRGGSDVKFTYRPSVPAHRALKESPLSSDNIFKQSHAGLFNLCIVVLVAVNSRLIIENIMKYGWLIKTGFWFSSRSLRDWPLLMCCLTLPIFSLAAYLVEKLACRKYISAPTVVFLHILFSSTAVLYPVSVILSCESAVLSGVALMLFACIVWLKLVSYAHTNFDMRAIANSVDKGDALSNASSAESSHDVSFKSLVYFMVAPTLCYQPSYPRTASIRKGWVVRQFVKLIIFTGFMGFIIEQYINPIVQNSQHPLKGDLLYAIERVLKLSVPNLYVWLCMFYCFFHLWLNILAELLRFGDREFYKDWWNARTVEEYWRMWNMPVHKWMVRHIYFPCLRHKIPRGVALLITFFVSAVFHELCIAVPCHIFKLWAFIGIMFQIPLVGITNYLQNKFRSSMVGNMIFWFIFCILGQPMCLLLYYHDLMNRKGTTESR

SEQ ID NO: 15 - protein, 521 aa, Jatropha curcas
MTILETTTSGGDGVAESSSDLNVSLRRRRKGTSSDGALPELTSNIVELESESGGQVMMDPGMVTEPETEKINGKDCGGDKDKIDNRENRGRSDIKFTYRPSVPAHRALRESPLSSDAIFKQSHAGLFNLCIVVLVAVNSRLIIENLMKYGWLIKTGFWFSSRSLRDWPLLMCCLTLPIFSLAAYLVEKLAYRKYISAPIVIFFHMLITTTAVLYPVSVILSCGSAVLSGVALMLFACIVWLKLVSYAHTNYDMRAIANSADKGDALSDTSGADSSRDVSFKSLVYFMVAPTLCYQPSYPRTDSVRKGWVVRQFVKLIIFTGFMGFIIEQYINPIVQNSQHPLKGDLLYAIERVLKLSVPNLYVWLCMFYCFFHLWLNILAELLRFGDREFYKDWWNARTVEEYWRMWNMPVHKWMVRHIYFPCLRHKIPRGVALLIAFFVSAVFHELCIAVPCHMFKLWAFIGIMFQIPLVGITNYLQNKFRSSMVGNMIFWFIFCILGQPMCVLLYYHDLMNRKGNAELR

SEQ ID NO: 16 - protein, 507 aa, Euonymus alatus
MAANLNEASDLNFSLRRRTGGISSTTVPDSSSETSSSEADYLDGGKGAADVKDRGDGAVEFQNSMKNVERIEKHESRVGLDSRFTYRPSVPAHRTIKESPLSSDAIFKQSHAGLFNLCIVVLVAVNSRLIIENLMKYGWLIRSGFWFSSRSLRDWPLFMCCLTLPVFPLAAFLFEKLAQKNLISEPVVVLLHIVNTTAAVLYPVLVILRCDSAFMSGVTLMLFACIVWLKLVSYAHTNYDMRALTKSVEKGDTPLSSQNMDYSFDVNIKSLAYFMVAPTLCYQISYPRTPYVRKGWVVRQFVKLIIFTGLMGFIIEQYINPIVQNSQHPLKGNFLYAIERVLKLSVPNLYVWLCMFYCLFHLWLNILAELLCFGDREFYKDWWNAKTVEEYWRMWNMPVHKWMVRHIYFPCLRNGIPKGVAFVISFLVSAVFHELCIAVPCHIFKLWAFFGIMLQVPLVLITSYLQNKFRSSMVGNMMFWFSFCIFGQPMCLLLYYHDLMNRNGKME

SEQ ID NO: 17 - protein, 511 aa, Lotus japonicus
MAISEDSESLFAAAAASSVIQSGSSVRRRPSAISAVATVEDESSSEEPVPVRDSGSDVDDSVSSEQHVSPATANREKNQVHDISATKFAYRPSAPAHRRVKESPLSSDNIFRHHAGLFNLCIVVLVAVNSRLIIENLMKYGWLIRTGFWFSSKSLRDWPLFMCCLSLAIFPFAAFVVEKLVQQKCISEPVVVLHIFISTAAVVYPVLVILRTDSAFPSGVTLMLFACIVWLKLVSYAHTNYDMRELTKSIEKGEALPNTLNMDYSYDVSFKSLAYFMIAPTLCYQPRYPRSPSIRKGWVLRQLVKLIIFTGVMGFIIEQYINPIVQNSQHPLKGNLLYAIERVLKLSVPNLYVWLCMFYCFFHLWLNILAELLRFGDREFYKDWWNAKTFEEYWRMWNMPVHKWMIRHLYFPCLRNGIPKGVAILIAFLVSALFHELCIAVPCHIFKLWAFGGIMFQVPLILITNYLQNKFRNSMVGNMIFWFIFSILGQPMAVLLYYHDLMNRKSKLDQS

SEQ ID NO: 18 - DNA, 1092 bp, Euonymus fortunei
atgatggatgttcatcaagagatcaataacttcatcaaggtttgggtacaagccatggcatgtctatcttacgcttactacttctcctcgagacttccaaaaggactcttaaggttactatctttacttcctgtcttttatctcctattaatccctcccttgaacatctcatctttcattctttcatcaatcaccggcttcttccttgcttggctaacaacattcaaggtcatctcttttgcctttgatcaagggcctttgtatccactcccacaaaacctcctacatttcatctccatagcttgtcttcccatcacagtcaagcgaaatccatcaccaaaattgaaatccacaacaaacacatctccaatttcacatattttgaaaagtgtattcatgtcatttccatctaaagtcttgttgcattggatcattgctcatctctaccaatacaaaaagtacatggatccaaacgtggtcttagtgatctattgttgtcatgtgtacgttctgttggatatatcactctctctatgtgcaaccctagctgaattcctttgtgggtttgatgttgaaccacaattcaaagagccttaccttgctacttcactacaagacttttggggtcgtaggtggaacataattgtgtcaagtgtcctacgttctacagtctataccccaacaaggcacattgcttcatatctaattgggtctagatgggcttactttccagctataattgcaacatttgtggtctcaggagttatgcatgatatagtgtactatgtgtatatgatgcatgtgtatcccaagtgggatatgacagggcacttcgtcctacatgggatttgtgaagctttggaggtggagatgaagtgtaagagatcgaggcgtgacaagtggcggcggcatccggttgtcgactgggtaatggtgatggggtttgtgatggggactagtgtttccttactctttgtgccactattaagggataatgtggaccaaattgtagcagaagagtgctcaattttgcttaattttgtgagggagaagattgtcatgcttggaacaaggtttatctgtggaaattga

SEQ ID NO: 19 - DNA, 1092 bp, Euonymus kiautschovicus
atgatggatgctcatcaagagatcaataacttcatcaaggtttgggtacaagccatggcatgtctatcttacgcttactacttctcctcgagacttccaaaaggactcttaaggttactatctttacttcctgtcttttatctcctattaatccctcccttgaacatctcatctttcattctttcatcaatcaccggcttcttccttgcttggctaacaacattcaaggtcatctcttttgcctttgatcaaggccctttgtatccactcccacaaaacctcctacatttcatctccatagcttgtcttcccatcacagtcaagcgaaatccatcaccaaaattgaaatccacaacaaacacatctccaatttcacatattttgaaaagtgtattcatgtcctttccgtctaaagtcttgttgcattggatcattgctcatctctaccaatacaaaaagtacatggatccaaacgcggtcttagtgatctattgttgtcatgtgtacgttctgttggatatatcactctctctatgtgcaaccctagctgaattcctttgtgggtttgatgttgaaccacaattcaaagagccttaccttgctacttcactacaagacttttggggtcgtaggtggaacataattgtgtcaagtgtcctacgttctacagtctataccccaacaaggcacattgcttcatatctaattgggtctagatgggcttactttccagctataattgcaacatttgtggtctcaggagttatgcatgatatagtgtactatgtgtatatgatgcatgtgtatcccaagtgggatatgacagggcacttcgtcctacatgggatttgtgaagctttggaggtggagatgaagtgtaagagatcgaggcgtgacaagtggcggcggcatccggttgtcgactgggtaatggtgatggggtttgtgatggggactagtgtttccttactctttgtgccactattaagggataatgtggaccaaattgtagcagaagagtgctcaattttgcttaattttgtgagggagaagattgtcatgcttggaacaaggtttgtgtgtggaaattga

SEQ ID NO: 20 - DNA, 25 bp, Artificial Sequence
  Synthetic primer
ggaagaagccggtgattgatgaaag

SEQ ID NO: 21 - DNA, 24 bp, Artificial Sequence
  Synthetic primer
ttggaggtggagatgaagtgtaag

SEQ ID NO: 22 - DNA, 30 bp, Artificial Sequence
  Synthetic primer
ttggagctcatgatggatgctcatcgagag

SEQ ID NO: 23 - DNA, 33 bp, Artificial Sequence
  Synthetic primer
tagactcgagtcaatttccacacataaaacttg

SEQ ID NO: 24 - DNA, 33 bp, Artificial Sequence
  Synthetic primer
tagactcgagtcaatttccacacataaaccttg

SEQ ID NO: 25 - DNA, 30 bp, Artificial Sequence
  Synthetic primer
ttggagctcatgatggatgttcatcaagag

SEQ ID NO: 26 - DNA, 33 bp, Artificial Sequence
  Synthetic primer
tagactcgagtcaatttccacagataaaccttg

SEQ ID NO: 27 - DNA, 28 bp, Artificial Sequence
  Synthetic primer
ttggagctcatgatggatttcaatcaag

SEQ ID NO: 28 - DNA, 32 bp, Artificial Sequence
  Synthetic primer
gaaccactgtgctggcgtcaatcctgttccaa

SEQ ID NO: 29 - DNA, 26 bp, Artificial Sequence
  Synthetic primer
caggtaccatgggaggtgaactgagg

SEQ ID NO: 30 - DNA, 27 bp, Artificial Sequence
  Synthetic primer
aattcccggggcttgtggcaaagattg

SEQ ID NO: 31 - DNA, 27 bp, Artificial Sequence
  Synthetic primer
acatttgtcgtagctggagttatgcat

SEQ ID NO: 32 - DNA, 27 bp, Artificial Sequence
  Synthetic primer
tgcaattatagctggaaaataagccca

SEQ ID NO: 33 - DNA, 28 bp, Artificial Sequence
  Synthetic primer
tcaggagttatggctgatgtagtgtact

SEQ ID NO: 34 - DNA, 27 bp, Artificial Sequence
  Synthetic primer
tacgacaaatgttgcaattatagctgg

SEQ ID NO: 35 - DNA, 30 bp, Artificial Sequence
  Synthetic primer
tcaggagttatgcatgctgtagtgtactat

SEQ ID NO: 36 - DNA, 27 bp, Artificial Sequence
  Synthetic primer
tacatgatgcatatgtatcccaagtgg

SEQ ID NO: 37 - DNA, 28 bp, Artificial Sequence
  Synthetic primer
atagtacactacatcatgcataactcct

SEQ ID NO: 38 - DNA, 30 bp, Artificial Sequence
  Synthetic primer
tcaggagttatgcataatgtagtgtactat

SEQ ID NO: 39 - DNA, 30 bp, Artificial Sequence
  Synthetic primer
tcaggagttatgcatgaagtagtgtactat

SEQ ID NO: 40 - protein, 8 aa, Artificial Sequence
  Consensus sequence
  MISC_FEATURE (3)..(3): Xaa is Val or Ile
HDXVYYVY